(19) United States
(12) Patent Application Publication
(10) Pub. No.: US 2023/0210973 A1
(43) Pub. Date: Jul. 6, 2023

(54) PROTEINS AND NUCLEIC ACIDS FOR EHRLICHIA DIAGNOSIS AND VACCINATION

(71) Applicant: Research Development Foundation, Carson City, NV (US)

(72) Inventors: Jere W. McBRIDE, Galveston, TX (US); David H. WALKER, Galveston, TX (US); Tian LUO, Galveston, TX (US); Jignesh PATEL, Galveston, TX (US)

(73) Assignee: Research Development Foundation, Carson City, NV (US)

(21) Appl. No.: 17/998,194

(22) PCT Filed: May 10, 2021
(86) PCT No.: PCT/US2021/031509

§ 371 Date: Nov. 8, 2022

Related U.S. Application Data
(60) Provisional application No. 63/022,365, filed on May 8, 2020.

(51) Int. Cl. A61K 39/02; A61K 31/7105; A61K 31/7115; A61K 31/7125

(52) U.S. Cl. CPC A61K 39/0233; A61K 31/7105; A61K 31/7115; A61K 31/7125; A61K 2039/53

(57) ABSTRACT

Methods and compositions for diagnosing and vaccinating against Ehrlichia canis and Ehrlichia chaffeensis are provided.

### BACKGROUND OF THE INVENTION

This application claims the benefit of U.S. Provisional Patent Application No. 63/022,365, filed May 8, 2020, the entirety of which is incorporated herein by reference.

### 1. FIELD OF THE INVENTION

The present invention relates generally to the field of molecular biology and medicine. More particularly, it concerns diagnostic methods and vaccine compositions for Ehrlichia.

### 2. DESCRIPTION OF RELATED ART

Ehrlichia chaffeensis (E. ch.) and E. canis (E. ca.) are tick-transmitted obligately intracellular bacteria that cause human monocytotropic ehrlichiosis (HME) and canine monocytic ehrlichiosis (CME), respectively (McBride and Walker, 2011). HME is an emerging life-threatening zoonosis in humans, with 50-70% of the cases requiring hospitalization and a fatality rate of ˜3% (Paddock and Childs, 2003). CME is a globally distributed disease in dogs and is the most serious form of canine ehrlichiosis (Harrus and Waner, 2011). Therapeutic options are limited, and currently there are no vaccines available for HME or CME. Progress in developing effective subunit vaccines for HME and CME has been hindered by many factors, not the least of which is the small and incomplete repertoire of molecularly defined E. ch. and E. ca. protective proteins (McBride and Walker, 2010).

Previous studies have identified a small group of major immunoreactive protein orthologs of E. ch. and E. ca., including the tandem repeat proteins (TRPs) (Luo et al., 2009; Luo et al., 2008; Doyle et al., 2006; McBride et al., 2007; McBride et al., et al., 2011), an ankyrin repeat protein (Ank200) (Luo et al., 2010; Nethery et al., 2007), and the major outer membrane protein (OMP-1/P28/P30) family (Ohashi et al., 1998a; Ohashi et al., 1998b) with linear antibody epitopes that have been molecularly defined. During infection, strong TRP-, Ank- and OMP-specific antibody responses are consistently generated in humans and dogs that can be demonstrated by immunoblot (Chen et al., 1997; McBride et al., 2003). Moreover, linear antibody epitopes of E. ch. TRPs and OMP-1 have been shown to stimulate antibodies that are protective (Kuriakose et al., 2012; Li et al., 2001). Although protective linear epitopes in Ehrlichia spp. have been defined, there are limited examples of conformation-dependent antibody epitopes, and there is little knowledge regarding the existence of such epitopes or their roles in immunity.

The established antigenic repertoire of E. ch. and E. ca. consists of ˜10 immunodominant proteins known to be expressed in Ehrlichia-infected mammalian cells (McBride and Walker, 2010). These proteins were identified using approaches that depend on linear antibody epitopes; however, it is recognized that identification of the complete repertoire of antigenic proteins can be limited by immunoscreening approaches and other factors such as the host cell environment, which influences pathogen antigen expression (Kuriakose et al., 2011; Seo et al., 2008; Singu et al., 2006).

Many obstacles have impeded attempts to define the E. ch. and E. ca. immunogenic proteins, including challenges in growing ehrlichiae in tick cells, lack of genome sequence information, limitations of conventional protein analysis approaches, and difficulties in studying conformational aspects of protein immunoreactivity. Clearly, there is a need for new and improved methods for diagnosing and vaccinating against Ehrlichia.

### SUMMARY OF THE INVENTION

The present invention, in some aspects, overcomes limitations in the prior art by providing new and improved methods for diagnosing and inducing immune responses against Ehrlichia chaffeensis or Ehrlichia canis. In some aspects, vaccine compositions and methods of vaccination are provided.

As shown in the below examples, highly immunoreactive polypeptides were identified, and the in vivo importance of these immunoreactive proteins in detecting E. chaffeensis and E. canis was verified using ELISA tests on human monocytotropic ehrlichiosis (HME) and canine monocytic ehrlichiosis (CME) positive sera obtained from patients and dogs. ELISA testing using positive HME sera obtained from patients revealed that the following proteins elicited significant responses, indicating that the following proteins can be used for example in diagnostic methods to detect infection by E. chaffeensis or E. canis or may be used to induce an immune response in a subject against E. chaffeensis or E. canis:

TABLE 1
Immunoreactive Proteins

| | | SEQ ID |
|---|---|---|
| | | NO: |
| Ech_0875 | MKIIGKILPTRLISTFLGTGYLPAWQNHWAAVLSLILGYVLFYLIYGMSY | 1 |
| | VSYGILVTGAVVASLFLKISLGLIVISIISIFVFQSNNSADDRSDIIVVQ | |
| | IALGQLLVVALSMPAILAIYNYLAIFYSKICQNIFMCPSWFNDFMHFLMF | |
| | FLIPFVFFNILDIIKPWPISSLQILYNNCFSIVFEGLVLVIYTLIVMYLV | |
| | AFICFDLTIRDAVALNSYIFTLLKFR | |
| Ech_0129 | MITSVLIFVIVALTIGLWVIDCDGIIRIDWLGYDIEVNILFTLFVIAVVF | 2 |
| | LLLILLVRFIFCFSRCVYRYKRDLQNKKMVLLEQGYMYLNCGDVERVEKI | |
| | IVKIGNFDHPSLFLLKGRVYFDTGKYILAEKYFTQFVKVVPVIDASLGIH | |
| | LLNVIMQIEDQIQQLSLLRKMLEIFFKQSWSAIFKLTIYRISRDWGNAIE | |
| | EMKKIIKLKINLPLPYNTQEMLNVFYYALAKQCYDIQKYDDGLRVLDNIK | |
| | NCSQQCSTAVTLLKAKFYIDTDKKRKAVNILEHEYRINPHPDIANFYLDI | |
| | MQHSSHAIHKLYSFNTGYYFSIYLIAQDAINSGEYDTAMKYLNHSFKTKT | |
| | YISLYFLVLKLKVLSQNYNELLYWTDKIAKDAIADKYWSCTKCKYTPTCW | |
| | HYECDGCKSFNTIIWV | |
| Ech_1065 | MSEIQVKAENLGGESILEAPIRVSVNVGDSVKQGDMLFIIETDKTSLEIV | 3 |
| | SPEDGIINEIFVVDEEIIQRGQVLCTINTVKSNAVKPSEGNTAHSTTVTV | |
| | ADDMQQFIQKKDAPSAMKIMEENVIDKSQVSGSGIGGRITKSDVLNYMKL | |
| | ASEEDNTKANSISSLSVVSEEKREERVKMSKIRQVIAARLKESQNTAAIL | |
| | TTFNEVDMKNVMDLRAKYRETFEKKYGIKLGFMSFFIKAVVLALKELPII | |
| | NAEISGNEIVYKHYYDMGIAVGTDKGLVVPVIRDADKMSFADLESTLASL | |
| | GKKAREGKLEVADMAGATFTITNGGVYGSLLSTPIINPPQSGILGMHSIQ | |
| | KRPVAIDDKTIEIRPMMYIALSYDHRIVDGQGAVTFLVRIKQYIEDPSRM | |
| | FLEV | |
| Ech_0678 | MKNYICVIVYFAVVTFNFDALANHFYLKGGYNLVGNYSDSFKSEYMGYKR | 4 |
| | FNIDINTAAGYQLSNNFFYEVKVRYANIKPAIQKLEYFEKIDLIDILQRI | |
| | IDKKVYISKIEDIFKINSVTTLINCGYDYVINNKLIVYLSYGVGIAGLLN | |
| | YQGFRSNVATHYGMSIQSEVGLCYLYSKKMNLCIGYDYLKNYWKYDTNKI | |
| | YDEDGNTVVYHFQDFQLNSHTVFVDFKVIL | |
| Ech_0207 | MLHITQNSDNIVHSFYIAIQKYFKFTNLKKHCNTFRFTNFYFNHTIKCRA | 5 |
| | ANYIIYMQSFIIYKHPKKINTSDGIVFVQDEFSFMAFIFSILFTFYNKLW | |
| | LLSFISIVILSGIYIMYNTLNLINLPIYLSVNLLYSLYIASSYPDWYQAK | |
| | LKKMGYKIHDVIFAENLISAKLKFKR | |
| Ech_0121 | MQHTAVPGMVAPTSVISAKHVVIKGLVYKHVKHYSIEEYKSQIKEFRESI | 6 |
| | TCFARMHMSYMYHMLHNTFVVRNGRIMLKSEIEQCLSKITSNIRLCAFVI | |
| | KIGIVDHVMSRLCRFYGSDSIKYCASHYHDPRVIDSILIGLYGASYSDFS | |
| | RMSYQVRSNIVYCVGKHGIAGVFKLHNSGFYSELLGMCYDFVHARGKGVK | |
| | LQELCDFMKLSCSIQLGQMYHMMVKVKCSIGDEQSDIRKLVSQECSVGYL | |
| | VYRSLLFGRYAYHVRKAFRHLYAPSDKNPVRTVSGLNIPHSLIRLNHRGI | |
| | FTKIEHCINAEKMSFNVFVVDIVRHIDKLLLHPREEVYIREDISTYCAIV | |
| | SSRYSTMGPDIDSSYBIL | |
| Ech_0673 | MIKIKASRYTKIBLIAIAIILSLWNVFFIIKLISSIPQBIKNLIFPYTAI | 7 |
| | LSISSLLLYGINIIYNLNIMLKYHQGKIELQKKSLFSYLGVIGEATSLLA | |
| | LNIISTIAILILPMNKPMIIAASIFNILSSLFVIEYASIFLNTDIKNBKQ | |
| | LKNTNKSTKYTTWSIINWSAALIISIANVSFTFTGYFIKBNANDIFKIIL | |
| | FTVYISIITSLIFMRNMRENDPSILLNAQVEPTRFNTDEEREBLLKBEEG | |
| | ISHYSR | |
| Ech_1128 | MIYKEKLTRVGEYILAYLSFILSTYIFLVLVNIIRYNSLAICVISLLRTN | 8 |
| | IFNVSTKKLIKDKCRDTKFSNMNCYLYGKPLNLQIFYGIFSFIRNFQNNT | |
| | LIIPNDSKCGFYTTLWDNPALBYTYTLTGSEYRNFFDILYENIICQCKLL | |
| | INYNRSVLNQBNKNTLVIIPIPNAREFSNEIRVRNISINKESSYEC | |
| Ech_0670 | MFLFKKATYKTKYYIALCANTIYFGVFLFAIFIQLIRFPSKLSNKDLLLI | 9 |
| | NVLMVLSARIMLCLLSSYELTCTBYTNDNDSLLYYKKAAYAAEVISTIIA | |
| | IIIQVIAVSQVAAGNLEIVSSKKTTIBTKGAVDFACILIKLLIASPLLVY | |
| | FNYRRMKDDKCAAYKKDAEILFYLSITSLVISFIAFIGKIINVLEQTQSF | |
| | ALFNTENBSNNGPTNFPLGPIIRISCIAASIIILTVILSIESSISSKMSD | |
| | TTIEYQGLSNANELSG | |
| Ech_0706 | MLFRSGPKTIGSAVEKLILNKCDKNBISKIEVLLFFNWNNIVGEEISQVA | 10 |
| | KPKKLSFLNAMNTGVLYLVVNNGGVAINIQYAIPIIIEKISVFFGFKVVN | |
| | IIKIRQQL | |
| Ech_0518 | MSSFKNLIIRTTGFVIGSLLLIFSINMLMRRCSKRSNNVSIAVILLFSIG | 11 |
| | ITVFFQIYGRNEBSLTFVNIKMYVMLLASSLIMFCIANLVLRKESECYSK | |
| | FREQNKKIYFLKIDIANLEIQLNIMKREIEWFFDNLCSDTKICVDIKQEY | |
| | LLPANKKFYDIGFLCKTAKFVTFSIALVVFLCESFYSVLSQKFDLVGLFM | |
| | YYVLISLFLFTFIIDRLALYSEKNYKSISQNSKSIIDTLNTELNVKKRMY | |
| | TVLLQEVSKYNILTEKELVNENNIRTLEKYKNKIFDILDRV | |
| Ech_1055 | MTPRIKNTIYILITIILSMVCLVYASVPLYSIFCKVTGYGGTVRKSNILS | 12 |
| | SSKTGNTTIKVRFNADINKQLPWKFYPELPYVFVKPGEQKLIFYRAENLS | |
| | DKDISGMAVYNVTPHKAGKYFNKVACFCFSKQTLFPYQKTVMPVSFFIDP | |
| | AIETDPETADVKLITLSYVFFKYKE | |
| Ech_0640 | MMPLLBTVREKEGSQQIVKLYRKNSLIFLRKVVMGIRKIATKSWYNIPKE | 13 |
| | QQVSMDIFRKPILSMTATGVMQYSQGYDIVYKEISFKNKLNVEVFYRNCL | |
| | LDIKWFSLAERRYBTRFIREKNRPVLCSYDGQIYSTNSKVAYVITLEGNL | |
| | ITBDBIVCDRTQGQTYFBSTLAAGMPVICAGLLSVSNGIIBYISNESGBY | |
| | KPGIGNLYNAVKLLDKVISLDCIICVIGFAVNEETKKLRTVYQARVKGSF | |
| | LVDMESVGKDGLTIPERYFS11RKDNDNYKKRLIQAANSHNVQAR | |
| Ech_0040 | MDSVSANHIRNILFLVLGAFFGLEFCFYLSGVLFILMVWGPDYLDFNAIN | 14 |
| | PSFSDFPDRIWPTVFNYVEBWWBNPSSYDAILLVKLISSLCIPIGILSIV | |
| | LWNLRNILFDWRPFKKKESLBGDSRWATEKDIRKIGLRSRKGILLGKDKR | |
| | GYLIADGFQHALLFAPTGSGKGVGFVIPNLLFWEDSVIVHDIKLENYDLT | |
| | SGWRKKRGQEVFVWNPAQPDGVSHCYNPLDWISSKPGQMVDDVQKIANLI | |
| | MPEQDFWYNEARSLFVGVVLYLLAVPEKVKSFGEVVRTMRSDDVVYNLAV | |
| | VLDTIGKKIHPVAYMNIAAFLQKADKERSGVVSTMNSSLELWANPLIDTA | |
| | TASSDFNIQEFKRKKVTVYVGLTPDNLTRLRPLMQVFYQQATEFLCRTLP | |
| | SDDEPYGVLFLMDEFPTLGKMEQFQTGIAYFRGYRVRLFLIIQDTEQLKG | |
| | IYEEAGMNSFLSNSTYRITFAANNIETANLISQLIGNKTVNQESLNRPKF | |
| | LDLNPASRSLHISETQRALLLPQEVIMLPRDEQILLIESTYPIKSKKIKY | |
| | YEDKNFTKKLLKSTFVPTQEPYDPNNVKITKKENEDPIPSIESDIPENKP | |
| | NNTENNLTEEGAIYNSTETNYEDDEDEDDDFNFDDLDEYIDEDEEYSEQP | |
| | EEDYDYTDEEDEDEEEDEEDNDYNNSNEDDDYENNNISNSHHENDFDNHN | |
| | EKPVKDQDIKNDKQ | |
| Ech_0720 | MYITRKVSINQFIALLSCCLICSIFLLKSVYKRLLTRKDENTPLPVEPEP | 15 |
| | IILNRYVPEYSTTILPEHYAANYSIHRNLCSISQNLKLLISQPYGISEME | |
| | SYPCRNHYLNVVHGSRDVLLFPKFRGYFDYILDKLICAGMFVEHNPVDIR | |
| | KVKDLLGEENFTFVILAAQLIIRNEALYATYSESAYDKIAMLYNLSIEEV | |
| | PYQERLNSANKIIRLLGNKVYSFSSQDELCKFLSNVFREYMHDCVSEIGN | |
| | ENDFYRMVNNDQKFKLLQEIFCMSYFKYYCIYGHRSHAVVTQEIDLASNI | |
| | IGQYDEPKAVDDEPIIERICNMRKSGFEVELIDNCCSVLNVQEIISQIFE | |
| | QTVIENNRRVSSFETDKVTRLLGTIEDVIEKVKEVTHYDAVDVVSSGRDY | |
| | YRLMINMAYSGPPYNRMFISKELSMIMNQEECLFSVFSSFYDYARIVDKV | |
| | TWSLADFEECEAEAANRFKDAEYYILYTRLKAKLDVGLFILHTRPDLPMT | |
| | QDQILKVWYNDDASQFGLDKHILKDVLRNIVCCARVVCCLPYLLHSYPEN | |
| | IVDYIRMLARLDIDIYCTANMIISYDSYDICVFKDQDEFQQMINSFLISY | |
| | NEYYKDLDTGLDNSRVTFGSSFDIVLVSLMYTSLYNSSLVEQHVIDAACN | |
| | RMKSFNFCYLERNYRSLMTIIAETVNCRHNSIYWPLCEMDLSNSIDMSQE | |
| | SNDESIGEKWYQHTILPVLGNSAMTRGSH | |
| Ech_0755 | MKKRHTDNTQNNKVDFVIRRHGLSVCKLAIFLIAPLLCISSLYLFNIYDS | 16 |
| | YINIIINCIFTSISILAISIQLRQYLYTIASIEYQNMIFANSLNHNTEFC | |
| | LIIKNNGKIIYADARFYERFIKNQERKLDLFDILKLGNISEQEIEAFENA | |
| | LKNKLSINTYFCLNTKNKISNFTLILDPVLENPEIEINYTKTVNLFFAPL | |
| | ARPQEYFVLKAVKITKEQVYERLIQKHCVGAYLLNHRGVILSVNDSFLKM | |
| | FELNSVENGTTFANFLTKSDNKEKVIDNNTVTLSTTTGITFKAHISQAAF | |
| | YDKNNHSYIYGLLTPVNSDILNYHLHPCFTEAPIAILQCNTDGKILKSNN | |
| | TLKRLVKHSKEYIFEYILPSYNKKIKKYFQNNIVKNMSLEAQLYNNSYVK | |
| | IHFNKFIHQNNMFIICYITDSEDRKNLEIQLEQSQKMQAIGQLAGGIAHD | |
| | FNNILTAIIGFCDLLLIRHTATDPSFSDIMQIKQNANRATNLIKQLLAFS | |
| | RKQTLQPKILDVNNIIADLLHMIKRLINESIELKIQYEQNINLVKADLCQ | |
| | LEQVIINLVVNARAAMETGGQLSIKTYNTKTDVLKVLLKDMFSPDKEQIE | |
| | DGEYVAIEVSDTGHGMEDKIMKKIFDPFFSTKETTAGIGLGLSTVYGIVK | |
| | QTDGYIYVKSIVNSGTTFIILIPTVHLSNNNDIEIPYNKYSNETSTTVPE | |
| | VNTSTTILLIEDEDPVRIFTTKALTKKGFKVIDINCGDQALDIINKYPID | |
| | IVISDVVMPKISGPEIVEQILKVKPNIQIIFISGYAEEVFNKYSNIDVSK | |
| | INFLSKPFTLKQLTEKVLEISDNSFLQN | |
| Ech_0947 | MIEIYSLLFVDSFVAALILPLNKILIFKIMAYFGGYSYPLMLLVSTLGAV | 17 |
| | IGGVINWVLGRMIIFARVEYHKVQDDYGKLGVYIKLALMLLTLLCSWIPV | |
| | WGGIVNVLSGYFRVEMLKLVVLLFLSYLGYLTYCIITL | |
| Ech_0044 | MIRMFNKFRKKNNKNDNNKLSNANLETTYSWHVSRYNSVVIQRNMLLFFT | 18 |
| | MLALSAVGISVFVIFNISKNRTIEPFVVEIEKKSGITTLVNPVSVKQYSA | |
| | DEVLNNHFIIEYVRSRELFDPNNFQYNYYTKVRLFSNQTTYSEFRNWIRL | |
| | SNPASPLNLYANVTSGYLKIRSLQHLRPGSVQIRFSLEFNHPSGIIKKDR | |
| | IATLSFQYVTLEMNEQERQINPLGFQITYYRADDEFL | |
| Ech_0988 | MNKKLLRRYCITKISVCVILLVVLSMTLHYLYKFNTYIVQHNLRIADEIQ | 19 |
| | KLSFKIMDVHKHEAMLNDSGVLWQEISTSNIYSTFYEEDLGTLISSLFKK | |
| | YYIFNSQINISSPKIVNNIYHNQHIDIIKRNIEIKFSSISDEQVFLFLNT | |
| | IRHDISGYVKVVNFNIEKNTDITNEVLQAALKGETVPVIRATILFELYNI | |
| | VGRFVDES | |
| Ech_0635 | MKYFNKKLIVISSIVIASISSLYIGVWLVIATHVKATLSKSLAYIHAEHS | 20 |
| | NDIQITNFPFIPKIKVLNLKINSSSLNISIPSLSLKYHLLSNTLEFIGLD | |
| | NATLTFENSMKITNNDQKNTSVCNLHDNFKLLIKPSKNLLLFLLDNKNDK | |
| | KAYFTSMVYEDNGISCNNNHIINKSIFSVETDEHKSFDNLITVLQYKINA | |
| | SAASNTDTNINVKIKDAIVTLSMSNTEFKEISAIIDNIALTSRNSLISIY | |
| | GKLLLPLSLDTQINDEKLIIEISNYQDIIKQLVKILYPKSQHKDKIFSAL | |
| | QDYIYAISEKKDNGNIILSVSETVNPFNIFICKVPYEEFISKIKAITNTM | |
| | DNHANKK | |
| Ech_0681 | MNIINVRRELSEQSKKDNSALSDARKQIAVACGFIGESTGFLGIASILLI | 21 |
| | LGSHLLPFSSIALPLRLFNIVASLFSVTYGSILLMKSIEGNARSKQCNSV | |
| | RIRLHAVWSLLNSVIFVSLGIYNLLYETLLHEVLDFSANHDLAMLLKAAL | |
| | FIGYAVILTSIFLSQVFVPSHDPVANKSSNTLESETATLLGQEGCEELCA | |
| | TLLEVDPINVNRGELDNNRCC | |
| Ech_1038 | MKVWCYKNIGLYLIVLLSFVYPRALLHAKINIHVLRDYANVHYNTYYGLH | 22 |
| | FDNYYKPVDNTEGNLDSVMIRFASHDSYRYMSFMQYVKGQYQDVENLNVS | |
| | GLDIPFNREDRTFDDHDVMFMQLSSYWGKVTFHSLPIGGCKVLYAGSIIY | |
| | DPVSAIAFLENENASSKVCICFVGKCNVKPERNSCDKKSIRCKKVTVAIN | |
| | PPPFCSILDASSIVSITPLRFSQQTFFRPGVRIHIYSGSGNPYTKELYVK | |
| | SHKIGDKTSYNISHAGIPYEFQVYKAGYDTVCAEYSNNGEKGKKVCVPSP | |
| | GLMRPKVTSNANGVNIQYQDCQGLSSCSVDMLPGTQDLDMYFSVIKPKID | |
| | FNNYTLLSRYECEDGKIVENENQCVNGIGQRLGYVHDNNSNVTCVVDMPF | |
| | VPMKYSIKKNHRDLWLSMHDKMLLGYGVVVGKTDTGKDVERYVQCDQKFA | |
| | IDIKSMTQEQLNKITRIRQDAFFDIGGHYNPKNAPCQDSMLYRYENNRLY | |
| | EKGGQVSCKNMVELDYGTKKIKGCSSLYMSDDDFTYFFHENDELEKIVPL | |
| | NPILQGMCVSNFPSYEYKKRVLVRKILPDSYKLGIDQKNTECDFLKIEAW | |
| | GGGASGISRSGRSGKAGNYVMGLLRFDKNVVNKKLIIDIGDGGKGANSLS | |
| | NSGGDTTVKLCDDDDKNCLVKIIAHGGDEGGNYLQDSSEGIDNLVHYRFA | |
| | PGLQNSGESEILVPYQSPDMPYGKLRKGDKECLCDSNILEKNSNKYWGAG | |
| | GCSSVYNCAQEGANGMVRITCEKWSGNVGKISLIDENACSDFLVTLIEKM | |
| | HKSTSGIPNVVKEFLQKISKVSFCRQSKSFPNLISSMSKYFIAIDKILVG | |
| | GDILGHNLSGLRKELFTELNNTEVKAMLAKLGINESPETLLLYLDVLNFN | |
| | FGVNISNPPSGLLNYYVSDNEFDYDLSKHDEDYDKLSDNEAMMFNVTTEK | |
| | PEQWFSVELKDPEFVRRYRNFINMIHRSVITDEEGHKKNNIVVSWMYTFF | |
| | KSDRQLFELYAAPFVELMLGMDLNKFMKWGNCSDTHIRLFESIGKYSERL | |
| | PSQIQDFIKKIATGDFCNTFSKMELLNSYGVELSNYAIDCALKNTDKRCL | |
| | ESKWRSSLGSMAKKLQDAVDLNYEVFTDIGIASNRKEIALLIDAVMLNYV | |
| | MSDLNVGNSDITSTLSLLDPISSQALDNFPYDTIVELRQDASNMKYGKYG | |
| | DHRANIVTLWSYMAYNSFEWNIEDFKSFVKLLLAEDLTSVKIRKCNDNLR | |
| | YLFDKLNKYKSKLPAVLQDFLDKISKESVCKKISRFAALETSLVKYEENL | |
| | LNELRGGNLFYFSSLYDLNYAHRGKLSNIEKVYSHAANIWSDVGELSSNI | |
| | TNLLNNPEIYKIFTDAGITSSKEEISLAFDAVIFNSLVSEIKIDQKKLKN | |
| | LLLLIYDNSLALNNIRLERSKGSGQIQQVTIDQNRYPGNGILMLQQNANN | |
| | MEYGNYGVHRADIIALWSYISYMSSESGWSIKKCESFVKLLLGIDLKFID | |
| | LKGCDNDVVDLFNKLNTYGDKLPLSLRDFLKKISEKNFCEKMSLFPELGI | |
| | ALMNYTNELRNVLRVGSVFQVDSVANIVNGRVSNINDVYSYLGNLLSNVR | |
| | QLSSNIADLLNNPDIYKIFTDVGITSSQEAIFLSIDAVIFNLLVSEIKID | |
| | NSQLKELLSLVGGHRNASSNNANNGRSLSQGIRYKITFKISFAQNYFPVD | |
| | EIIKLQQDANNMEYGVHGVHRTDIIALWSYISYASSKSKWLFKRYQSFAG | |
| | LLFEIGAWGKCTSAERVFFTSMNRYSEKLPLKVYNFIRKITTGDFARKFS | |
| | GMQSLYTYKQRVYDYVMHCVLRGGLGGECSDMTLREISNELHKLKQEIYS | |
| | NYDVFRDLGITNGQQEVLLLINVMMLNYAMSDLLVSTTQVNSMLADVRSS | |
| | LSRTAHCLPYGTVSQLQRSVSHMKYGEYSNYRASVVALWACISCLASFND | |
| | DMEPLIKLMLEGD | |
| Ecaj_0151 | MKAIKFILNVCLLFAAIFLGYSYITKQGIFQTKHHDTPNTTIPNEDGIQS | 23 |
| | SFSLINQDGKTVTSQDFLGKHMLVLFGFSACKSICPAELGLVSEALAQLG | |
| | NNADKLQVIFITIDPKNDTVEKLKEFHEHFDSRIQMLTGNTEDINQIIKN | |
| | YKIYVGQADKDHQINHSAIMYLIDKKGSYLSHFIPDLKSQENQVDKLLSL | |
| | VKQYL | |
| Ecaj_0128 | MMRLLACLGIVAVIIVAFNFLTNKQQVQDQKQEVHVYSSRKEELLHSLFE | 24 |
| | QFTKETGIKVKYINDEAAQLINRMENEGTATSADVFLTADAVNLILAKKK | |
| | GLLQPIQSEVLNQAIPSKYRDSEGFWFGLTKRARVIVYNKDLVEDNELST | |
| | YEHLANTKWKDKILVRSSSSPYNQSLIAFMVANNGVENTKIWVKGLVANM | |
| | ARKPSGGDIDQIYAVAADEGSIAIVNSYYFGRIAASDKKSDQAVVKKLGI | |
| | FFPNQETTGTMINISGGAVTKHAKNKQNAIKLLEFLTSVRAQKVYAQVNQ | |
| | EYPIVEGVELSEVLKTFGTFKESDLPLQELEKHLTKSVEIADECGWR | |
| Ecaj_0213 | MVLQNDNITDDTQNNKDEVHDTQQSTEIVKESSVVTSETQPTHDKKLDKI | 25 |
| | DFNTLFLAISFAGLLIEAASAIFNLVSTYVYVPERIKHIVATAFYAISII | |
| | VSLSMMASSILAIKQSLNSKKKLQETSTGPNKEAERSINEGLLGYDKLKQ | |
| | KQANIQISENTITIISEILWIIVSAASLVMITVGTGTPALELASLCLAVI | |
| | APFLAFISCALRLSDANISRKTATSNKEKRHASNFTVLCTIILLFEAIHC | |
| | GCHVAEAVMLGGKMQNIYDFQDAIVLGLELAAVVMFIAAFFIEKYLDKKA | |
| | EKSDPQATPSSLLDDKAIDRMFREAQIS | |
| Ecaj_0162 | MVEERKPHINVGTIGHVDHGKTTLTAALTTVLAKRLSGEGNKSVKYDEID | 26 |
| | KAPEEKARGITISTAHVEYETENRHYAHVDCPGHADYIKNMITGAAQMDA | |
| | AILVVSATDGAMPQTREHILLAKQVGVKDIVVWMNKCDVVDDEEMLSLVE | |
| | MEIRELLSKYGYPGDDIDVVRGSAVKALEEETGSGVWSEKIMELMNALEK | |
| | ISLPVREKDKPFLMSIEDVFSIPGRGTVVTGRIERGVIRVGDKIEIVGLR | |
| | EIQSTVCTGVEMFHKALDAGEAGDNAGILLRGIKKEDVERGQVLSAPGQI | |
| | HSYKRFKAEVYILKKEEGGRHTPFFSNYQPQFYVRTTDVTGNIKLPEGVE | |
| | MVMPGDNINIEVSLDKPVAIDQGLRFAIREGGRTVGSGIITEILE | |
| Ecaj_0554 | MAVIGIDLGTTNSCVAVMEGGDAKAIENSEGARTTPSIVAFTDSERLVGD | 27 |
| | PAKRQATTNAKNTIYASKRLIGRRYQDVKDIKSSYEVVSAKNGDAWIKVL | |
| | GKEYSPSQIGAFVLEKMKETAERHLGHKVEKAVITVPAYFNDAQRQATKD | |
| | AGKIAGLDVIRIINEPTAAALAYGLNKSDKQKVIAVYDLGGGTFDVSILE | |
| | IADGVFEVKATNGDTMLGGEDFDHAIMDYLMDDFKKTTGIDLHNDAMAVQ | |
| | RIKEASEKAKIELSNRMETDINLPFISSDSTGPKHLSLKLTRAKFENLVD | |
| | DLIQRTIEPCKKALKDAGISADKIDEVVLVGGMTRVPKVIQKVKEFFGRE | |
| | PHKGVNPDEVVAIGAAIQGSILAGDVRDVLLLDVTPLSLGIETLGGVFTP | |
| | LIERNTTIPTKKSQVFSTAEDGQTAVTIKVYQGERKMAADNKLLGQFSLE | |
| | GIPSAPRGMPQIEVTFDIDANGIVHVSAKDKASGKEQAIKIQSSGGLSDD | |
| | EIQRMIKEAEQKAGEDEKRKKFIELKNNGENLVHSTEKSLNEYGDKIPNS | |
| | DRLEIENAIRDVRDALGNSDVESVDILQQKVDHLMKVSMKLGEALYGNAN | |
| | NTSSTESTTTNNNNEEDSKVVDSDYQEIDKKDGK | |
| Ecaj_0857 | MSEIQVKAENLGGESILEAPIRVSVNVGDTVKQGDMLFIIETDKTSLEIV | 28 |
| | SPEDGVIGEIFVTDEAMIQRGQVLCTINTVQSSAVESSDTSSAHNATTTA | |
| | ADCMQQFIQKKDAPSATKLMKENSIDRDQISGSGVSGRITKSDVLNYMKS | |
| | TASEGSNINRLAVVSEGKREDRVKMSKIRQVIAARLKESQNTAAILTTFN | |
| | EVDMKNVMDLRAQYRENFEKKYSIKLGFMSFFIKAVILALKELPVINAEI | |
| | SGNEIVYKHYYDIGIAVGTDKGLVVPVIRDADKMSFSELELTLAALGKKA | |
| | REGKLEVSDMAGATFTITNGGVYGSLLSTPIINPPQSGILGMHSIQKRPV | |
| | AVDDKTIEIRPMMYIALSYDHRIVDGQGAVTFLVRVKQYIEDPSRMFLEV | |
| Ecaj_0334 | MKSKGLFVKPLLIILVCSLVFIAFGTSFFPGNFNNDKYVAKIGHEKLSLQ | 29 |
| | DYTNAYHDELRYIQQILNRPLTEEQIAQFNIKLSVLNKLIENKVLTKFTD | |
| | SLNLKVGEKSILSHIKSIKFFQDENGNFDKTKFNIGLSNAGLTERLYINK | |
| | LEKAFPVAMLMSCLFSGTQNTYVKYHPELIKQILQNLHQARTIDLIEISP | |
| | STNDISVPSLDELKKLYEEKRKSGNLTFPENRVIEYLTINYKNFINQINV | |
| | TQEDIDNEIKTEELDEQRDILNLVFSTKNEAEAALKALNEGTDFNDVVTN | |
| | IAHTTTDNITLNNIVKNTLPNDIRGKVFSLKEGDISPILHSMFGWHIIKI | |
| | KSIHKISTEDLQELSEKIAQNIKKKKAISLLTTEIESINDKINNGASINE | |
| | IAKLYNLSIKTINTDISGKDLSGNKIKMSESNADAIILAAFSAQLNKPSN | |
| | FTDNDDYFFSVNVTKVEPSREKTFDESKEQLVYEWQNDLKSQKLSKLTQE | |
| | VVVKLKSGIDIHNIDGVLLNSNQMVYRNSVAAVDNPGQNYPTDLVDEIFN | |
| | LKKGEISKSYQSSTNSSEQKMLIAILKDIKNADQVSNVDFKNIQEKIAND | |
| | NLESLKNQLITYLTKKYSVKINQNLIDNVR | |
| Ecaj_0104 | MLGKDYFSDLDKRLVAFAKLNNGKQRSSKYHFMLSSCILITAIITVLSLI | 30 |
| | VLYSDKQYFSSMFKGGFKLFSSDTIPFSMALIAIIPSLLLLFFLIYKVCA | |
| | FHDLNRKLNNESIDILGKLEEWQYFLHLQLEYNTNKIENIDEVLKLFKKK | |
| | YSSFVKQCSDGFAKLEEQCKSVLAAVNSTTHGVNAGISKINCVVEDVKLQ | |
| | LNGLSAGCQKFSESSSNLLSAIESAIKTEGVNVDDRIAFLQNLQANLASD | |
| | VDLGLIQNRVQNFVARVRSILVEKPSIVKPAVLRELWCLQESFNVIIMKV | |
| | HKTDQCALEFLQLIQNLEQEISKFSLSASKKIASYNGNNRNLLEKMIKLE | |
| | LPLLHINILRFLTAKRAAFITNTSADITQFIQEYASGDLQRLSDSTSDSM | |
| | LSISSFLSLQDTNVSDKVSNDINPQ | |
| Ecaj_0737 | MGNLDIQKYVYLALFLLLLVILLMLAIYRYCVASEHDLQRQDVLVLSREY | 31 |
| | YNQLAGGYRQLKNDQSVMFVHNVRLAKTCEAVAEEKRNLEKKIKELGDDM | |
| | SEVSVSMAVELNSLGVHPTVVSSMTSQMLSLQRRIYDVARSSPVSIVKAD | |
| | QGVQTEQQQASSALDQILASGLQCNTGGKVVH | |
| Ecaj_0179 | MNILSVDNVQDLRNLHAISHPIEKIDQEIIALANDMMKVMEDSKTVGLSA | 32 |
| | VQLGNHSRMFTINMFSGLFDVTQDIKVLSGHHSLHGKNMVCINPEVLSFS | |
| | AETVDLFEGCSSAKSYGLINITRPKHMDFRYTDLLGNKCVVRVYGWLSRC | |
| | IQHELDHLNGILLANVVDNIKNNCVNSISYEDHSVIHILLVNKK | |
| Ecaj_0589 | MIFDEDSNSVTQDSGYMTVGNAYSNAVGYISMSDHWSKLTKPSSIKVESN | 33 |
| | GASPNKADLIVEPLESGFALTLGNALRRVMMSSLRGFAVYGVEIENVLHE | |
| | FTSISGVREDVTDILLNISMMRVKLSGLSNKVLSLRVKGPCEVRSGMIPD | |
| | TDDCIILNKDLLICTLDQDVDFNIKMYVNSGKGYVPAVKRKSVSKLSDVP | |
| | VNFIATNALYSPIKKASFKVESSRIGQFTDYDRLVLSVETDGSILPDEAV | |
| | ALAARILQDQFQPFINFDETDEPHKKIDTKDALPYDSNLLRKVDELELSV | |
| | RSYNCLKNDNITYIGDLVQKTESDMLRTPNFGRKSLNEINELLASMNLHL | |
| | GMKIANWPPESIESLSKQYSEE | |
| Ecaj_0805 | MFVKLKLNDDLESNTTTTSDNTHDNQDHNQTCATKSKNIISVTATEDETT | 34 |
| | SDDSYNTQELTPQQITQALDRFIIGQADAKRAVAIALRNRWRRNRVPEPL | |
| | REEIIPKNILMIGHTGIGKTEIARRLAKLAKAPFIKVEATKFTEIGYVGR | |
| | DVDSIIRDLVDVAINLVKEKFRKIVEKKAKALSESMILDALIGPDASEET | |
| | KTIFQEKLRNGEFEDSEISISIKESKNTMPPIDIPNIPGNQVGIMNINEI | |
| | VHKMLGNNKQLKTIKVTVKEARELLINEESEKLMDEDKIIKEALQLASND | |
| | GIVFLDEIDKIAARTEIRGEVNREGVQRDLLPLLEGTSVTTKYGTITTDH | |
| | ILFIASGAFHLAKPSDLLPELQGRLPIRVELKPLSKDDLVRILTEPESSL | |
| | LKQYCALMKTENITIDFTDEGVCTIAEIASTVNREVENIGARRLHTILEK | |
| | LMEDISYTATENSGKTYVIDSEYVKQKLEDISKQLDLSKFIL | |
| Ecaj_0851 | MVLFMKAHSTSIRNFQPLERAAIIIAVLGLAAFLFAAAACSDRFQRLQLT | 35 |
| | NPFVIAGMVGLAVLLVASLTAALSICLTKSKQVTQHAIRHRFGYESSTSS | |
| | SVLLAISIISLLLAAAFCGKIMGNDNPDLFFSKMQELSNPLVVAAIVAVS | |
| | VFLLSFVMYAAKNIISPDKQTHVIILSNQQTIEEAKVDQGMNILSAVLPA | |
| | AGEDIMTIASCDILAVSSRGSSQHQ | |
| Ecaj_0728 | MDMNKGLLVAIAVLALFVLLLLFALYIAYRKYKVCSNQMNCLSEELIVGE | 36 |
| | KRYNTALGKLEKEVHCLTKQLAALRNENAKMLLALEQSEKSEVAFEHDSA | |
| | LERQLQLSNKQMVLKNCAKLKDLASRLRTEVSEAQARHEDDVMQEEHLLS | |
| | AAFNSMVSCYKSMLGNSCHQQVKEAMKDLESSERCVKKHLYVLCDKLGEG | |
| | FISAEEDISDEVFNPVIQIVNHAQKKVA | |
| Ecaj_0850 | MRNILCYTLILIFFSFNTYANDLNINIKEATTKNKIHYLYVEHHNLPTIS | 37 |
| | LKFAFKKAGYAYDAFDKQGLAYFTSKLLNEGSKNNYALSFAQQLEGKGLD | |
| | LKFDEDLDNFYESLKTLSENFEEALVLLSDCLFNTVTDQELFNRLLAEQL | |
| | AHVKSLYSAPEFEATTEMNHAEFKGHPYSNKVYGTLNTLNNLNQEDVALY | |
| | EKNSFDKEQEVLSAAGDVDPTQLSNLLDKYLLSKLPSGNNKNTLPDTTVN | |
| | REDTLLYVQRDVPQSVEMFATDTVPYHSKDYHASNLFNTMLGGLSLNSEL | |
| | MEELRDKLGLTYHSSSSLSNMNHSNVLFGTLFTDNTTVTKCLSVLTDLLE | |
| | HEKKYGVDEDTFALAKSSETNSFLLSMLNNNNVSELLLSLQLHDLDPSYL | |
| | NKYNSYYKAETLEEVNKLAKKLLSNELVLLEVGKNNNLNGKQLDAKKHLL | |
| | G | |
| Ecaj_0746 | MDNYCELLCAALVLLLALLMAVLFGLLYKSTRDLKRSLESQSKSYNSELG | 38 |
| | LLKQSLESLKANEAGLNLQNENSVLNRVNTYDKKLELLKNDLKGFSSKCN | |
| | SLETACDTLKKQLESAHGELASLSKKEHEAWKDKNKDGTVALPSQNQLFD | |
| | EDLMALVEELNEMKMAVAKLMSQNETHDLMNLEHSVSNLVCSVSSLSDSL | |
| | SALESERVEYSEYLKNLAAKVASNLDEETFRVSAEERKESDLH | |
| Ecaj_0818 | MKTSSDNSMEVTRMKYQMDKMGENARELAHKADVGKSFVYDLLSGKSTNP | 39 |
| | TSKKLMALAKVLNVSLSYLESDDNYTYGQGNMNLLPVYDLELENGKLSSS | |
| | GDVNLYLSSNENLTSNMKNLRVYYVKGDSMEPTLMNQDVVLVDLGDKLPH | |
| | PAGLFVIVDTVGVSIRRLEYLRENQKIKLHVVSDNKKYSSYECHLEDMEI | |
| | LGRVIWYARSL | |
| Ecaj_0882 | MPNNNDYDTYSKFERDLRSTVFYLRNRGVLHLGDLVSNVTPATYLLDSAK | 40 |
| | EGLREQFLSRVFVHHSNNKNDEKKVLDFLLSLGFLPVVNADYKVTEHEGV | |
| | CLYDKNRKYGDKHVLEVSSVVRHTTRDVRNLLLESRKNFRSSFDDGAKDS | |
| | ERTVLYDPDSKPLKQRLYDALSYSEKWSLLTTQEKVLRCLFTVLSLSLPL | |
| | IPPLLLLSYIGNIDIREAAYSMNLLPWQYRVYMKTSDSLYDVIVNNSDEE | |
| | RRKMETDNEMETAMAKGFVVKGEGDELQPTDLGKHVSGKHNLLLVAHNKE | |
| | LSKKQLLLRETTFSVLAFVCFSQLLALTCHLVRAEKLALYCSLLSYFVLF | |
| | VEGLYLLSLKTTMSKVCATFCLVFAGFMLMVHAFLLYNDLTFGYGPLMSG | |
| | LMVEETGAVLSVALACNQYYKQDRDLALKLGGLSTTAYLSMKRDLDGLDE | |
| | VKTVKLEGGCGYQEEDDLSLALFNAYNAGVFGTVQDQTVGNRQDELYSGT | |
| | QENDNCPNTSIGDLQSIDRSNSQDLNL | |
| Ech_0991 | MDMINIFDNTEDDAFSVSNFINQNFISQFTITILPPSVPLYHDQHIDEGM | 41 |
| | YSVVFSYKKYEAQQPYGLVEHKSGKFEASLDHSDHRLYLNKDDISIVLNE | |
| | DMLNLCLSCTKVIDNKDSAQ | |
| Ecaj_0126 | MTSNTSEPKNHEYSFKLDLGENLYFFCNHNVHKVKLLTEDNTELTMPSKN | 42 |
| | YFFVGDKFYAPYNNYFYDNYLNEPAEYRYEKVDHMQYRTTNNEQPQDFYN | |
| | LVLCDKNGEEYRYNYYKFYEKPENELEKSAELNLKEYYNLQQLKEGAPLF | |
| | KLVSEQPNNTTKASTALELDESSNQKFAKLSPEALQYKHYLDRNSPTYDT | |
| | FTLSYSDLRKHHVDEQEKINLHNIRDDILQAEMENNPIFLVIQDGKYFFT | |
| | DVKQDQPLTTSYNTALKVLASANFQINNVPNDNCYVDMHKKFIFKITKSN | |
| | LHTEHDNSKNLASITLEGKEIPLISNDDDTQIFYDDFSFKCYQNFTQVFN | |
| | YDEPIIGLDKDFYEPIKEKLSSNNIYITIKSDEQNHIKTYFSDKQGNHIL | |
| | DLPNTKLTEYLSTMLPLGDFSNEVLNTHIEDIAHQKLSDTTQKHDTLNPE | |
| | KNSTTLQNSVNETAGTNDPQSTQNAVHKHDTLDTQKDSTTSQKSVNDTAS | |
| | TNDSQSTQNAVHKHDTLDTQKDSTTSQKSVNETASTNDPQPTQNAVHKHD | |
| | TLDTQKDSTTLQKLVSEEHNINKSNTNINVEQNIVYFPLSREHVSIVDNI | |
| | EQNKHHVSFNLTYEEMLNFYEAVKEQYSYDEVLIAYNNIFKNYGREQKND | |
| | NIYIDGDNHIFIENHDFGILQ | |
| Ecaj_0920 | MDIFSNELNATVHVNGTTYEGKVIIDNNGNFDTNLSLADGVDTLGHLCGN | 43 |
| | ISQNNETKENSYILEYIFEQRIVYPTLPILHSFNGQIVSSAEEALPHQIA | |
| | FDNSNDNIKIILSDSEIVQPVTNAKESQAEVSKPVTDVKENQDGAPQPAA | |
| | NTPQEKQESVPTPADGVNNDPTKEGASQPNKT | |
| Ecaj_0259 | MFGFLKKGASVIIKAAVTPTTSKLPHQESVGKHLEGMLKSIVPGQKSSRE | 44 |
| | KNFDLKMHDRTYKLGVELPGRTRVAGHETEVALKVPSYKLPYQALQKFAA | |
| | WQEQNKEGEVDETRKDALASLITPVAATRELIASKTADQYFQKLKELDSQ | |
| | LKIICGKVTLDSTAGSYKQGLTVELDVSGKSKEQIEQEVKIVLQSLGITD | |
| | SKLAKTFAQRLYKTSTDPKVSSADIQDKKTNTDSKDTTDDKDVVSSAPAQ | |
| | NSDVVSPKKEQRSDLYPAPESSTMSDSDFSLTNPRNMPYNTRGPLQSSEM | |
| | LQKLQQQILMQHRPEPLFPQPMSPSMQQAVEEIARDLPTNINEEEHMQGN | |
| | STNTGIPIHNTGKAHARR | |
| Ecaj_0922 | MSIDYDSCQIDFSIQRSGHPGVDVYHGIMHVNEEGSSYYASGHIVNDSGN | 45 |
| | EGIVNIIKYCNRGIYNDEHCTECDLEVILDDPNSSRYVFHVSKDDTCLMC | |
| | DERNILSMMKPSRKVQIQNDAVVALDIYGCKNT | |
| Ecaj_0271 | MFYPKRDLKTIYSKDNTLSAEHLLELMNSSSKELKEYRSQLLTGYVKKEE | 46 |
| | KKLTDQNKTPEEIQKHISSPEFFYKASSYADYKVNHGIEMFLISQIGNKV | |
| | KILNQRIKSLGYDRQSMILHGMYKISLLTEINSIIKDMSDDDFKELLEVI | |
| | ISCSHLKTDLTEQQQQSRQAAVAEYVKESKGFSMEKVREKAEELTGIDIA | |
| | NPIASLRKKFGADKPEGKQHMVSMDEKLVQTVINPFNSYYFILGALAIGL | |
| | EGFIPPLVPILLIAITGKSLFSLVTEDKDKEGSVHSPFQEGIIRQPVTAE | |
| | GQQNYTAMLERQKKEESEQQSTSPTSKKL | |
| Ecaj_0767 | MGNSGISIIVCVVFIFLIMMVLVSMFQRNASIFDCVSRQQCKASNADHDK | 47 |
| | GNDTSNQSEEASVNTDLVSGSNGTCEDIDLQSVQSRKCCRT | |
| Ecaj_0096 | MVALYTPQVDKNVIAHDFSLKSTDGNLYSLSSYHKGNGLLIMFICNHCPY | 48 |
| | VKAIINNLVYDVNTLKNDYNIYTIAIMPNDTIAYPDDSYENMIKFAQEYG | |
| | INFPYLIDEDQTVAKNYGAVCTPDFFGFNNKLELCYRGRFDSSGKNQVDS | |
| | KQEDRDLFNAMKFISKTGKAPDNQKPSIGCSIKWKSQDDQFVPH | |
| Ecaj_0730 | MCIVMLMLLLLIAMCQCCISMRTLDGKFESEAMAEQRIQELVDENQNLVL | 49 |
| | ENRELSAACKSANGKLSELLLQMKNLLLSAETQSEKILNDLALDNQANLC | |
| | TKKAVQCGVLKIRQGIFDIVHNNREEQRRNIELGGNTPSKIPVSKLRMQS | |
| | LSTLQSENKVQSCCN | |
| Ecaj_0736 | MDTTAIVLLVLLIVMLLLLVLLAVAAVKCQKQYNFLKEENKKLLACCEEA | 50 |
| | SDYSQELVSQNDKLKKESERIKSKSDKLERELKNKMQKFFSDQVSEYEDL | |
| | KESCIELINIRGESVKKLFDDVKQDMSPESVKCLIDTNILFLQKSITYEI | |
| | DSSKNTLEKRLSDQFPSNDIEGASVSSTGGNVQPNDIA | |
| Ecaj_0717 | MLGDEIAAIVIGVLLIIVSAILLYRLICWMLTPELHVKKGIPSHIDVQQT | 51 |
| | KFDSEETKPQTVTLPKVRTAGYLQFMLEAHDCCFNIVLDMMKQSANNKML | |
| | VGDNLTFLMDKICNSSSKLKELQNLSIGSEAALEQYLPTIYQCCLALVKY | |
| | GSLIQSQIKKVSRKTCNTEQLIPYQLTVSVLMHECNMLLREIKQEMVNAT | |
| | ESDRLPVSILESSTANSHSTSTGLNQ | |
| Ecaj_0748 | MYRDIFCIIVAVAVVLVILILLCIIGYMISCIKSLGNQIDKMKTELSQCR | 52 |
| | KEDTEMMEEVLTLYDEVYDMRNDLAYHPYFGNVKNGKKLGNEVGDTTLQQ | |
| | GSCTLDSTNVKGLGDKCEKDK | |
| Ecaj_0348 | MNKSQSLYLFLFLLCVTILAFTAAILAFRFNDRLKIPYLNIIAAVVMSVL | 53 |
| | AVALLISVAHLVMHYQGTSVSVKDMPPRIAAGDQMVLFVPFTADQLSKLS | |
| | PKNNIIDVKYDVVESGSSLAGKREFVLDITQSYGLCPTKKVQVVMDNNRQ | |
| | CQLLFTNEEISSQVNGPGMCFVVNGNTSIVKGNMQKPGFYIKHPEVKDGT | |
| | LSLNCEYQKICTGSMEDLRYILRSECVRSGRTIYEMLLSNILTMCPNQKD | |
| | KVIPSDASVTHSKDLWINTNSGRLALSLMKIISPDSTLINLEELKNDDEP | |
| | LYQKITWIRNYLVTNGHNVSEASFEVIFKKASDIFYRRTAGMSEYGNLHS | |
| | DRITLSGKKFSDILAEGMQLDEELLIIMAIASYSCEGTLDPYNTTNKFLR | |
| | EILMLDQQRSDSILKEIEEILGKHWYEIIYLNDLKFSGSKVCTQMKKLLQ | |
| | DGCDSATALEIVNAARAYMLEKQWSSVPITRGVGCIMNLGYLDRIRLCGN | |
| | REVSVETQEEGVSSSIVEVVDTSSVQNLLAPGNMQ | |
| Ecaj_0676 | MCILGADKIIPPHIPAVGTGKGYSSDGIAIPRNSKEILKNKITPGTKPLV | 54 |
| | VFIGGALDDTTRLVLRLYARYYTQNHDHQDIAYATWGSSATIPIIKAWYE | |
| | AEQKICLVGHSWGGDSEYKIVKKLEMNTIDLMCTIDPVSRAGVGGKLPKP | |
| | KNLKKWYNVAIDYKKVKFSINNLIAQTGGPWFYCMYADKNFIVDTVEINK | |
| | HLVSADHAMAEALFFKYFNEYVKNFATQE | |
| Ecaj_0636 | MADDEYKGVIQQYINTVKEIVSDSKTFDQMFESVVKIQERVMEANAQNDD | 55 |
| | GSQVKRIGSSTSDSISDSQYKELIEELKVIKKRLLRLEHKVLKPKEGA | |
| Ecaj_0347 | MEVNYKTIIGIVIGIAIAALITIAVLTVLNVIPIAVLLQLSTVFASAIAG | 56 |
| | SVVVYAKEKIFAWYDKQQYEDRASTSFKMVDKVLSASDEKIAKSSDKVVL | |
| | SNDTISNYSNYPFFPEFHKNFNKILDKLSLDVRFASINQELFYSSLSPQE | |
| | QTLAILSARLICKQPEMYLIYSEPVRVCIAAIAQLKIDKKFEGRARNNII | |
| | SLLRKQVRISNYNVLEQELKGVMLKHTKDPVFMKLLHGLIDIDEFSGKKQ | |
| | IMNLVNLFSSYKYYSIYSDSAVEYYSDMASELLKQINNKQESAVDVDAKS | |
| | SNVPKHKTSDNEKSIVDKRTSNSSVNACDTNNQELENNFSSKITRINSTK | |
| | MRGL | |
| Ecaj_0723 | MCERSLNVIIALLALLALLIVVLLCAVIYKCHVDCQNERQRSSEIEEMKL | 57 |
| | LLRGQPQAILEPDVDREKIEKLCEEKFKNQLKEQQKANSDLQSKLKSLEK | |
| | TNKHLAEVVSSLKKHGVIINRKNTELTEQCSMLRKNCAQLGVVCRKLGYD | |
| | NKVLMRDAQVVFNAGFYSFKEAVDGMLQISAEIELLLLKRNERIDALTVG | |
| | EIEYHLLTLRTILMDLEKQEKKSQKQPTQQVKAASVLPCSDQDNNKAA | |
| Ech_0159 | MLIDQKFQHLSQAFNSYSTLYQRNKLKSKYNFLLGSSIYLTALVSVFSIV | 58 |
| | ALYHHSGCFNSIFSGKDKLFGGNIPVVFSIALIVAIPSFILLFFLIYKIC | |
| | ATQDITDHVVERTNDLIGSAREVCEFLKEKLHENTCQVEALVGHYGSLAA | |
| | KHDDFVKKCSRCLQSLEEQQVALGALVRRSTSVVDNNISEVQALVVDLKA | |
| | KIAEMSVAHVKLMDASEDVREFIVKQLRLEGLGLQHRVRFLDGLQGTLSQ | |
| | NIDVSDAKSAMKIIIERIENEIPKVRFAFNSLNSMLWSLHSNLLKALLGL | |
| | DKDSSPDGCITVCQELVNALHKFLGDMRAKIDNYNGSDKRQFVNIGLVEV | |
| | PLLQIMILRLMATKRACMFEDVCASVKDFIESGTADRSSSTGTKVIPDSA | |
| | ASAAAPVTPTASVTPAASVTPATPTTPAASVTPIASATPATPAASVTPAA | |
| | SVTPTASATPATPAAGTNDGAEFATPPMGTTDAPSVEDLSALGAVGGVDP | |
| | NAHTHTP | |
| Ech_0251 | MRDKVKDLSNNNNSRNSNPIWNILRSIFTTLGNWGNMLANVLPRILISGI | 59 |
| | VPPIIPTNATNNNPQHNENSSTTHSNENTQSSDHNNMEQHTSQSEIPVLQ | |
| | SSANLDSLSDIEETPRRNNEDEIPVWESSTDSDGISDTENMPIDSHEQHD | |
| | SDTVSNDTNSDIEFISEEDVLEASGFFIVDICDNPNSSVTHIDLQSGSQD | |
| | MVYRP | |
| Ech_0531 | MIHNGNKKHSATNALLNVATVVSALVMTFSAFCTIYSGLNLAFNIKIPGT | 60 |
| | PQTSMGLFLAFVLTFTAGSLSLISAAVANRLSQPFVLTQETSRNTSQQNT | |
| | SEDDQHRLRSTSQQNTSEDDQHRLRNTSQQNTSEDDDQHRLRNTSELDGQ | |
| | DNNSPPSSVVRQSTMQGIPSAQQEV | |
| Ech_0285 | MLSLLIYVMSKKCYMTAAITVGEGEDIQSVQAPERNLSNGVSNPRVVNSI | 61 |
| | DSSSNMSADSASDMGADSASNMSADGAGNMGADGAGNMSADGAGNMSADG | |
| | AGNMSADGAGNMSADGAGNMGTDGADSGSDIDTDNSIGSDVIEGGTSGDE | |
| | EEELHSVDQHMVNVGNNGVIAVEPQSSICFR | |
| Ech_0147 | MVALNTPPVNKKFIAKDFLLKSIDGNLYNLSSCHKGNGLLIMFICNHCPY | 62 |
| | VKAIINNLVYDVNTLKNDYNIYTVAIMPNDTITYPEDSFDNMINFAQEHS | |
| | IDFPYLIDENQTVARNYGAVCTPDFFGFNNKLELCYRGRFDASGKNQMNS | |
| | KQEDRDLFNAMKLISKTGESPENQKPSIGCSIKWKSQDYKFVT | |
| Ech_1148 | MDIFGNEFDVHVNANGTEYAGKMSIDSNGDFDVNLDLQDGVGTLGHLSGH | 63 |
| | ISQSDDAANYIIEYIFDQCIIYPELPVLHSFNGQIVSPAEEGSIIFDNGD | |
| | NIQISLHGLQEQPEEAIPAAEVEEAVPAEAAIPAAEVTENQQ | |
| Ech_0259 | MNNTNITNSTQSNNSTNSDNSVTIYAPIIAITCLFILMIIICRRMHRNRE | 64 |
| | NQVSTTAPSRSTSFEIRRHTNFTNYVLYDSFYEPELTDREVMALVDVDAP | |
| | HNNTRRHNNSNSTNASGR | |
| Ech_0704 | MAGHSQFANIKHRKGAQDAKRAQKFTKLIREIIVAAKQGLPDPEFNSRLR | 65 |
| | SAIIAAKKENLPKDRIDAAIKSATGNTQTDNYEEVVYEGYGPGNIALMIQ | |
| | TLTNNRNRTAAELRHALSKYNGKLGESGSVSFLFNHVGVIAYKASSIDSF | |
| | DSLFNTAIELHALDVEEIIQDDTQEKIYYVICNVQDFGNVRDQLFHKFSD | |
| | AEFSRLFWKASDTVKIEDEEIQKKISNLMDLLENNDDVQYIDSNFTFY | |
| Ech_0281 | MSEGLIVVIALQVFLLLLLFIFALYHYCIKPCSKSGKMGYERFEGENIPK | 66 |
| | YEGVLEHFEEMDYLQLQQRVAELRLACRMLDKERVTNDRTYEECLDWMFF | |
| | VSLTLDELMVENKLESDYITRMRAQYHLFNLRIIADQVARQPAANVERQE | |
| | DLQEVEQSPSPQVEGTSVASCCGQEKQAA | |
| Ech_0478 | MRNVKYVLFWLGFIVLVMLFSGTKDDIGGNKYFNKFFPNAATQNNKVNYV | 67 |
| | EGGVEFYRAKDGHFYIEAMIHGIPVNFLVDTGATDVVLSVEDAKRLKHHL | |
| | KYLNKKKTYHTANGTVKALYVEISEMQVGKFVVNNVKASVNVSPMRTSLL | |
| | GMSFLQYFHFNMSGDKLTLHSY | |
| Ech_0715 | MNKTQSLCLLLTCFLFFTAAALVYYFRKDISTTPYLHVAAILLMVLMAIM | 68 |
| | AIAMLVIVAHMAMMCSQSYTKIQSNLPSIKEGKEVLVFVPLTEDTVSRLN | |
| | FGGGDIFKIECNTRASVMNLGQGNGKGIVLTLTRKYGLCSQRTVDVQVDT | |
| | NRKCAMLFNDQQAANVKGPGLYCVTKLNEKSGVFQDRLKSDTSLTVEYVE | |
| | VHGGKVKVWHRYCKRTLGQNFASFLLSKKVEDKSHTASDDDDTISLYEVM | |
| | LRGLLRSCPSSSTLIFPSDDCDENSEAWVNRDDGKLALSFLMSFSPASIL | |
| | STLKNLKDKEVLEKVQLLQRYISQCDLTPDDEHLPIICERIKYKFFETHE | |
| | KISSYNNIEDRLTTSGSSFFTLLRTKIEEDKEGILPGVLAIAAHQFQNDL | |
| | APDTKSNMFFVGILSEQGKHGMIKEVRELFGKNPDQTLIGDPYSLIAFDS | |
| | CKLIGLQISPALKLMGSVLGPTCTAQELVDKARGYVMDIYWKNIVVPNMV | |
| | LDCVEFGNAQNVSLCAGQDMALENSSDNVSIISGANVCCFGSCDPNPYSR | |
| | I | |
| Ech_0181 | MVMSYFDYILSYIINNPKYTALLVLVLLAGIAYLFKRKKSKSKYKKSTTS | 69 |
| | YTNNNSSSSTFTPKPHNKNYIPEHSTQHSQGSNFFVVESVSQVFESKMQG | |
| | RSR | |
| Ecaj_0923 | MGLYYNEYQNDHQIDFLIQQNGHSNVDIYHGTLHINGNEGKYYASASIVN | 70 |
| | DLTGDKGTINILNHGVKGIYSNESPKCDLEITLEHYDPASSDNIITSINK | |
| | SFIVSVFSNLQDCLLIDSHNPLLMDPSKVEQINKEPVIAVLVHDGIDTKK | |
| | IRDSFFNEKPENKEHKAGGCTYVYSHGKTETKCE | |
| Ecaj_0071 | MQSDTHESIVRVDVSNGSNDEQLNDNSEFAKRTDKSTQKTKGSDRKSECL | 71 |
| | LKDIVESSEKTLVTESTSAYSQDIHDAEEFESRLSSSQWSTVQKGTRSTT | |
| | KGDKEQKKFSGDGVKCLGKTEGVYSESVVESSAQRVSVPKGSIVENSSQW | |
| | DKKNQDIKNSQLKLLKAENIAESGIISGSGEEWSVKFSKKDSKRNKKSQT | |
| | FESDATVKAIDVSHASTVGEGKASQELSSKYLQQGARPKVRSHTQSISVG | |
| | LPIGNANSKDVSQKKNVSLSTVNVSEMKAHSCMVTGIGTGVHLETKPIGR | |
| | SGDTYGCVTNKQYLASVDKFKMSVEKFAKMHMQYIANIVFDVFAIENGNI | |
| | IFRDEIQNVLLKTTKNIELCSFVIKQGIIMQVMSRVCSSKLYNSVMYCAS | |
| | HWDNQKILDAVLLEILNLGPNALVSMLPSMRNVFLNMHEGIEHNVFRLYS | |
| | GGFYSGLLNMCYDFITQIDKSPGLMRLYNFIILSSQIHFGTMYDVICKSQ | |
| | VTAAGVQNVEAIKKLIFNGHRVGSVQICPTIFVHRCCGVKRTFRHLYSPN | |
| | YKNVACTIGNLNIPSVIIKECSCNIDEKVAQCIEEKTMSFDVFVEDMVCN | |
| | CVKSLLYQNVKLLLEKDIALYEKEVRSKYQTMGSTTSSRML | |
| Ecaj_0220 | MLNRTQNDNITQTLQGANNSYSPEENANNLNIEEQLINNPEIQVNPNIQA | 72 |
| | EEVIADAIQEPYNPNAQIEAANATEDNIINDTIEENDNNLNIEEQLINNP | |
| | EIQVNPNIQAEEVIADAIQEPYNPNAQIEAANVIEDNIIINNTIEENANN | |
| | LNIEEQLINNPEIQVNPNIQAEEVIADAIQEPYNPNVQIEAANVIEDNII | |
| | INDTIEENANNLNIEEQLINNPEIQVNPNIQAEEVIADAMQEPDNPNAQI | |
| | EAANATAITNINAMVTLQNINEIYPEDPESLITQEERAIEYMIQKREQVL | |
| | AEANIYNDISRPRVLIDHSAAMQDILKGYSIAKTQNKINNIEQEILRNTI | |
| | IQAEKNVGILIENRKDLHVNTTQEEPNAQIETDITDAIQQEELEEPSNQS | |
| | NDISNNSSHVSSDNTETVEVDIEAIPSLLRRIARDIVALINTELNDPNAE | |
| | HVNTQNQNINK | |
| Ecaj_0824 | MSNTNDFQSIKDVIANIRKVMSSNDNGDSTTEQHDTNTQPDDTEHEVLNL | 73 |
| | ENPENHVELHNIQHAHNTLNEINQTFQAISDLPITTYQNKTQPDIQISVK | |
| | KEKIYPDQLSNVNQFISIEQSTQRLSSQERRLTTTHTLSEEISKNTSEIK | |
| | GVHQKNTFISENLVSPESIVASSEEIKKLITQVHNYTKPTNVSSDKSPTI | |
| | EELVINMLKPELSTWLNNNLQKLVKEIVEKEIKHIIKKSNQN | |
| Ecaj_0342 | MIKTHDIAINTISAIMVTAAATGLALCSIGIIREHQYEYCALCVLLLLSA | 74 |
| | CVSLIANYKKGISRKCASIRNYLSKISSDESLDGENKYYDPAPDLFAEHY | |
| | FDEDAPSADRAEKIHVDRRQYYSEPYENEEQGQYSAYEEALHHSGWIRSQ | |
| | PFFAEYDTTPPDNRSAEMKTFSSSSSEAKVDTTIRGMQDKNNSKIQITDS | |
| | RVEAVKPLSEEQGKGRA | |

As shown in the below examples, all of the polypeptides listed in Table 1 demonstrated significant reactivity with sera used for screening. As shown in the below Examples, the proteins listed in Table 1 displayed an optical density (OD) of at least 0.2 or greater. In some embodiments, the protein is a protein of Table 2:

TABLE 2
Medium
Immunoreactivity
Proteins

Ech_0159
Ech_0251
Ech_0531
Ech_0285
Ech_0147
Ech_0949
Ech_0259
Ech_0704
Ech_0281
Ech_0478
Ech_0181
Ech_0715
Ecaj_0736
Ecaj_0730
Ecaj_0071
Ecaj_0923
Ecaj_0220
Ecaj_0824
Ecaj_0342
Ecaj_0767

As shown in the below Examples, the proteins in Table 2 showed reactivity to the tested sera, and ELISA OD values between 0.2 to 0.5 were observed. Even more preferably, the immunoreactive protein is a protein as shown in Table 3:

TABLE 3
Highly
Immunoreactive
Proteins

Ecaj_0126
Ecaj_0717
Ecaj_0636
Ecaj_0920
Ecaj_0259
Ecaj_0348
Ecaj_0271
Ecaj_0096
Ecaj_0748
Ecaj_0676
Ecaj_0922
Ecaj_0723
Ecaj_0347
Ech_0991

As shown in the below Examples, the proteins in Table 3 displayed 100% reactivity to all the sera tested and had an optical density of ≥0.5 with least 4 sera. In some embodiments, it is anticipated that a protein having at least 90%, more preferably at least 95%, 97.5%, or at least 99% sequence identity to a protein in Table 1 or Table 2, or more preferably Table 3, that retains at least some of its immunoreactivity can be used in various embodiments as described herein (e.g., in a diagnostic test, or to induce an immune response against Ehrlichia in a subject, for inclusion in a vaccine composition). In some embodiments, the protein may be used to generate an antibody that selectively binds the protein, and the antibody may be used, e.g., in a diagnostic assay. For example, in some embodiments, the antibody is labelled or attached to a solid substrate (e.g., in a lateral-flow test). In some embodiments, the protein is Ecaj_0126, Ecaj_0717, Ecaj_0636, Ecaj_0920, Ecaj_0259, or Ecaj_0922; as shown in the below examples, these proteins continued to react with dog sera even after denaturation of the immunoreactive protein. Without wishing to be bound by any theory, these results support the idea that these immunoreactive proteins (i.e., Ecaj_0126, Ecaj_0717, Ecaj_0636, Ecaj_0920, Ecaj_0259, and Ecaj_0922) have linear epitopes, and the linear epitope may be comprised in a larger polypeptide with additional Ehrlichia antigens or epitopes. In contrast, after denaturation, Ecaj_0348, Ecaj_0748, Ecaj_0676, Ecaj_0723, Ecaj_0736, Ecaj_0730 and Ecaj_0767 exhibited markedly reduced or no reactivity with most dog sera tested, supporting the idea that these proteins have conformation-dependent antibody epitopes. In some embodiments, it is anticipated that one or more of these immunoreactive proteins that may contain conformation-dependent antibody epitopes can be used to stimulate B-cells (e.g., in vitro, or in vivo after the immunoreactive protein is administered to a mammalian subject). In some preferred embodiments, an “Ecaj_” polypeptide from Table 1, Table 2, Table 3, or Table 5 can be used in the diagnosis of, or to cause an immune response against, E. canis. In some preferred embodiments, an “Ech_” polypeptide Table 1, Table 2, Table 3, or Table 4 can be used in the diagnosis of, or to cause an immune response against, E. chaffeensis.

TABLE 4
Highly Immunoreactive
Proteins Against E. ch.

Ech_0875
Ech_0129
Ech_1065
Ech_0678
Ech_0207
Ech_0121
Ech_0673
Ech_1128
Ech_0670
Ech_0706
Ech_0518
Ech_1055
Ech_0640
Ech_0040
Ech_0720
Ech_0755
Ech_0947
Ech_0044
Ech_0988
Ech_0635
Ech_0681
Ech_1038

As shown in the below Examples, all of the proteins in Table 4 exhibited a mean ELISA OD of greater than 0.9 using HME patient sera. In some embodiments, a protein of Table 4 may exhibit a mean ELISA OD of greater than 0.6, 0.7, 0.8, 0.9, 1, 1.1, 1.2, 1.3, or greater than 1.4 using HME patient sera. Ech_0875, Ech_0129, Ech_1065, Ech_0678, Ech_0207, Ech_0121, Ech_0673, Ech_1128, Ech_0670, Ech_0706, Ech_0518, and Ech_1055 were observed to be immunodominant.

TABLE 5
Highly Immunoreactive
Proteins Against E. ca.

Ecaj_0151
Ecaj_0128
Ecaj_0213
Ecaj_0162
Ecaj_0554
Ecaj_0857
Ecaj_0334
Ecaj_0104
Ecaj_0737
Ecaj_0179
Ecaj_0589
Ecaj_0805
Ecaj_0851
Ecaj_0728
Ecaj_0850
Ecaj_0746
Ecaj_0818
Ecaj_0882

As shown in the below Examples all of the proteins in Table 5 exhibited a mean ELISA OD of greater than 0.5 using CME patient sera. In some embodiments, a protein of Table 5 may exhibit a mean ELISA OD of greater than 0.6, 0.7, 0.8, 0.9, 1, 1.1, 1.2, 1.3, 1.4, 1.5, 1.6, or greater than 1.7 using CME patient sera. Ecaj_0151, Ecaj_0128, Ecaj_0213, Ecaj_0162, Ecaj_0554, Ecaj_0857, Ecaj_0334, Ecaj_0104, and Ecaj_0737 were observed to be immunodominant.

An aspect of the present disclosure relates to a pharmaceutical composition comprising a nucleic acid comprising an open reading frame encoding a polypeptide of Table 1, Table 2, Table 3, Table 4, or Table 5 formulated in a lipid nanoparticle or a viral vector. The nucleic acid may be a ribonucleic acid (RNA). The RNA may be an mRNA. The mRNA may be a non-natural RNA or a synthetic RNA. The mRNA may be an in vitro transcribed (IVT) mRNA. In some embodiments, the nucleic acid is an mRNA further comprising a 5′ untranslated region (UTR) and a 3′ UTR. The mRNA may comprise at least one analogue of a naturally occurring nucleotide. In some embodiments, the mRNA is chemically modified. In some embodiments, the analogue is selected from the group consisting of phosphorothioates, phosphoramidates, peptide nucleotides, methylphosphonates, 7-deazaguanosine, 5-methylcytosine, and inosine. The mRNA may comprises pseudouridine, a 5′ cap analog, or a poly(A) tail. In some embodiments, the 5′ cap analog is 7mG(5′)ppp(5′)NlmpNp. The chemical modification may be a 1-methylpseudouridine modification or a 1-ethylpseudouridine modification. The RNA or mRNA may be comprised in a pharmaceutical composition. The mRNA may comprise a 5′ untranslated region (UTR) and a 3′ UTR. In some embodiments, the mRNA is comprised in liposomes, lipid nanoparticles, or a viral vector. The liposomes or lipid nanoparticles may comprise an ionizable cationic lipid, a neutral lipid (e.g., DSPC), sterol (e.g., cholesterol), and/or a PEG-modified lipid (e.g., PEG-DMG or PEG-DMA). In some embodiments, the RNA encodes the polypeptide of Table 1, Table 2, Table 3, Table 4, or Table 5 for secretion. In some embodiments, the RNA encodes the polypeptide of Table 1, Table 2, Table 3, Table 4, or Table 5 as an intracellular protein. In some embodiments, the polypeptide of Table 1, Table 2, Table 3, Table 4, or Table 5 is comprised in a fusion protein. The fusion protein may comprise a transmembrane region. In some embodiments, nucleic acid is a DNA. The viral vector may be an adenovirus, or an adeno-associated virus (AAV).

Another aspect of the present disclosure relates to a pharmaceutical composition comprising a polypeptide of Table 1, Table 2, Table 3, Table 4, or Table 5 and an excipient. The composition may further comprise an adjuvant. The adjuvant may comprises a triterpenoid saponin, a sterol, and/or an immunostimulatory oligonucleotide. In some embodiments, the triterpenoid saponin is Quil A. In some embodiments, the immunostimulatory oligonucleotide is a CpG-containing ODN. In some embodiments, the CpG-containing ODN is 5′ JU*C-G*T*C*G*A*C*G*A*T*C*G*G*C*G*G*C*C*G*C*C*G*T 3′ (SEQ ID NO: 75), wherein “*” refers to a phosphorothioate bond, “-” refers to a phosphodiester bond, and “JU” refers to 5′-Iodo-2′-deoxyuridine. The composition may comprise an E. canis bacterin or an E. chaffeensis bacterin. The E. canis bacterin or the E. chaffeensis bacterin can be a heat-inactivated or chemically-inactivated bacterin. In some embodiments, the chemically-inactivated bacterin was inactivated with formaldehyde, formalin, bi-ethylene amine, radiation, ultraviolet light, beta-propiolactone treatment, or formaldehyde.

An aspect of the present disclosure relates to a method of detecting antibodies that specifically bind an Ehrlichia organism in a test sample, comprising: (a) contacting an isolated polypeptide comprising or consisting of a sequence of Table 1, Table 2, Table 3, Table 4, or Table 5 or a polypeptide having at least 95% sequence identity thereto, with the test sample, under conditions that allow peptide-antibody complexes to form; (b) detecting the peptide-antibody complexes; wherein the detection of the peptide-antibody complexes is an indication that antibodies specific for an Ehrlichia organism are present in the test sample, and wherein the absence of the peptide-antibody complexes is an indication that antibodies specific an Ehrlichia organism are not present in the test sample. In some embodiments the polypeptide is selected from the group consisting of a polypeptide of Table 2 or, more preferably the polypeptide is selected from the group consisting of a polypeptide of Table 3, Table 4, or Table 5. The Ehrlichia organism may be an Ehrlichia chaffeensis organism. In some embodiments, the Ehrlichia organism is an Ehrlichia canis organism. The step of detecting may comprise performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a mass spectrometry assay, or a particulate-based assay. In some embodiments, the step of detecting comprises a lateral flow assay or an enzyme-linked immunoassay, wherein the enzyme-linked immunoassay is an ELISA. In some embodiments, the isolated polypeptide is Ech_0991, Ecaj_0126, Ecaj_0717, Ecaj_0636, Ecaj_0920, Ecaj_0259, or Ecaj_0348. In some embodiments, the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055. In some embodiments, the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

Another aspect of the present disclosure relates to a method of identifying an Ehrlichia infection in a mammalian subject comprising: (a) contacting a biological sample from the subject with an isolated polypeptide comprising or consisting of a sequence of Table 1, Table 2, Table 3, Table 4, or Table 5 under conditions that allow peptide-antibody complexes to form; and (b) detecting the peptide-antibody complexes; wherein the detection of the peptide-antibody complexes is an indication that the subject has an Ehrlichia infection. In some embodiments, the polypeptide is selected from the group consisting of Table 2, or more preferably the polypeptide is selected from Table 3, Table 4, or Table 5. In some embodiments, the step of detecting comprises performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a dipstick test, or a particulate-based assay. In some embodiments, the subject is a human or a dog. In some embodiments, the isolated polypeptide is Ech_0991, Ecaj_0126, Ecaj_0717, Ecaj_0636, Ecaj_0920, Ecaj_0259, or Ecaj_0348. In some embodiments, the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055. In some embodiments, the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

Yet another aspect of the present disclosure relates to an isolated polypeptide comprising or consisting of a sequence of Table 1, Table 2, Table 3, Table 4, or Table 5, wherein the isolated polypeptide is immobilized on a surface of a support substrate. The polypeptide may be selected from the group consisting of Table 2, or more preferably the polypeptide is selected from Table 3, Table 4, or Table 5. The support substrate may comprise latex, polystyrene, nylon, nitrocellulose, cellulose, silica, agarose, or magnetic resin. In some embodiments, the support substrate is a reaction chamber, a well, a membrane, a filter, a paper, an emulsion, a bead, a microbead, a dipstick, a card, a glass slide, a lateral flow apparatus, a microchip, a comb, a silica particle, a magnetic particle, a nanoparticle, or a self-assembling monolayer. In some embodiments, the polypeptide is comprised in a kit. In some embodiments, the polypeptide is produced via peptide synthesis or in vitro transcription and translation (IVTT). In some embodiments, the polypeptide is recombinantly produced. In some embodiments, the isolated polypeptide is Ech_0991, Ecaj_0126, Ecaj_0717, Ecaj_0636, Ecaj_0920, Ecaj_0259, or Ecaj_0348. In some embodiments, the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055. In some embodiments, the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

Another aspect of the present invention relates to an isolated polypeptide comprising or consisting of a sequence of Table 1, Table 2, Table 3, Table 4, or Table 5, wherein the isolated polypeptide is covalently attached to or bound to a detectable label. The polypeptide may be selected from the group consisting of Table 2, or more preferably the polypeptide may be selected from Table 3, Table 4, or Table 5. The detectable label may be a fluorescent label, a radioactive label, an enzyme label, or a luminescent nanoparticle. In some embodiments, the luminescent nanoparticle is a luminescent rare earth nanoparticle, a luminous nanoparticle, or a strontium aluminate nanoparticle. The polypeptide may be comprised in a kit. In some embodiments, the polypeptide is produced via peptide synthesis or in vitro transcription and translation (IVTT). In some embodiments, the polypeptide is recombinantly produced. In some embodiments, the isolated polypeptide comprises or consists of Ech_0991, Ecaj_0126, Ecaj_0717, Ecaj_0636, Ecaj_0920, Ecaj_0259, or Ecaj_0348. In some embodiments, the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055. In some embodiments, the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

Yet another aspect of the present disclosure relates to a kit comprising: (a) the isolated polypeptide described herein of above, (b) an anti-dog or anti-human secondary antibody linked to a reporter molecule; and, (c) an appropriate reagent for detection of the reporter molecule. The polypeptide may be immobilized on a membrane or a microtiter plate. In some embodiments, the reporter molecule is selected from the group consisting of luciferase, horseradish peroxidase, a luminous nanoparticle, P-galactosidase, and a fluorescent label. In some embodiments, the luminous nanoparticle is a strontium aluminate nanoparticle. The kit may further comprise a dilution buffer for dog or human serum. In some embodiments, the kit comprises a lateral flow immunoassay or a lateral flow immunochromatographic assay. In some embodiments, the kit comprises an enzyme-linked immunosorbent assay (ELISA).

Another aspect of the present disclosure relates to a method of inducing an immune response in a mammalian subject comprising administering to the subject an effective amount of a pharmaceutical preparation comprising an isolated polypeptide comprising or consisting of a sequence of Table 1, Table 2, Table 3, Table 4, or Table 5, or a nucleic acid encoding a polypeptide sequence of Table 1, Table 2, Table 3, Table 4, or Table 5. The nucleic acid may be a RNA, such as a mRNA, or a DNA as described above or herein. In some embodiments, the nucleic acid is an mRNA. The RNA may be an mRNA. The mRNA may be a non-natural RNA or a synthetic RNA. The mRNA may be an in vitro transcribed (JVT) mRNA. The mRNA may comprise at least one analogue of a naturally occurring nucleotide. In some embodiments, the mRNA is chemically modified. The analogue may be selected from the group consisting of phosphorothioates, phosphoramidates, peptide nucleotides, methylphosphonates, 7-deazaguanosine, 5-methylcytosine, and inosine. In some embodiments, the mRNA comprises pseudouridine, a 5′ cap analog, or a poly(A) tail. The chemical modification may be a 1-methylpseudouridine modification or a 1-ethylpseudouridine modification. In some embodiments, the mRNA comprises a 5′ untranslated region (UTR) and a 3′ UTR. The mRNA may be comprised in liposomes, or lipid nanoparticles. The liposomes or lipid nanoparticles may comprise an ionizable cationic lipid, a neutral lipid (e.g., DSPC), sterol (e.g., cholesterol), and/or a PEG-modified lipid (e.g., PEG-DMG or PEG-DMA). In some embodiments, the nucleic acid is a DNA. The DNA may be comprised in a viral vector. The viral vector may be an adenovirus, or an adeno-associated virus (AAV). The method may comprise administering the pharmaceutical composition as described above or herein. In some embodiments, the polypeptide comprises or consists of a polypeptide of Table 3, Table 4, or Table 5. In some embodiments, the subject is a human or a dog. In some embodiments, the pharmaceutical preparation is administered subcutaneously, intramuscularly, nasally, via inhalation or aerosol delivery, or intradermally. In some embodiments, the isolated polypeptide comprises or consists of Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737. The method may further comprise administering an Ehrlichia bacterin to the mammalian subject. The method may further comprise administering an adjuvant to the subject. In some embodiments, the polypeptide is comprised in a multimer or fusion protein.

Yet another aspect of the present invention relates to a method of treating an Ehrlichia chaffeensis infection in a subject comprising: (a) contacting abiological sample from the subject with an isolated polypeptide comprising or consisting of a sequence of Table 1, Table 2, Table 3, Table 4, or Table 5 under conditions that allow peptide-antibody complexes to form; (b) detecting the peptide-antibody complexes; wherein the detection of the peptide-antibody complexes is an indication that the subject has an Ehrlichia chaffeensis infection; and (c) administering a therapeutic compound to treat Ehrlichia infection in the subject. The polypeptide may be selected from the group consisting of Table 2. In some preferred embodiments, the polypeptide is selected from the group consisting of Table 3. The step of detecting may comprise or consist of performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a dipstick test, or a particulate-based assay. In some embodiments, the subject is a human or a dog. The therapeutic compound may be an antibiotic (e.g., doxycycline). In some embodiments, the isolated polypeptide is Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.

Another aspect of the present disclosure relates to an antibody that selectively binds a polypeptide of Table 1. The polypeptide may be a polypeptide of Table 3, Table 4, or Table 5. The antibody may be a polyclonal antibody or a monoclonal antibody. The antibody may be a mammalian antibody. In some embodiments, the antibody is a humanized antibody. In some embodiments, the antibody selectively binds Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737. In some embodiments, the antibody is present in a multimer.

A method of detecting an ehrlichiosis infection in a mammalian subject, comprising: (a) obtaining a biological sample from the mammalian subject, wherein the biological sample is preferably serum or blood; and (b) performing a polymerase chain reaction (PCR) amplification that can selectively expand a nucleic acid encoding a polypeptide of Table 1; wherein expansion of the nucleic acid indicates that the mammalian subject has ehrlichiosis. The polypeptide may be a polypeptide of Table 3, Table 4, or Table 5. In some embodiments, the polypeptide is Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.

In some aspects a polypeptide of Table 1, more preferably a polypeptide of Table 3, is present in a multimer, fusion protein, or a nanoparticle. For example, the polypeptide may be expressed in a fusion protein with one or more additional Ehrlichia antigens, e.g., a TRP protein (e.g., TRP19, TRP36, TRP47, TRP120, TRP144) or OMP. TRP proteins are provided, e.g., in Luo et al., 2017. In this way, the fusion protein may be able to protect against more than one cellular target of E. chaffeensis and/or E. canis. In some aspects, the polypeptide may be fused to a ferritin nanoparticle, e.g., as described in Swanson et al., 2020.

In some aspects, an antibody can be generated that specifically binds a polypeptide of Table 1, more preferably of Table 3, using methods known in the art. For example, the polypeptide may be injected into a mammalian subject (e.g., a rabbit, rat, or mouse) to generate an immune response in the subject. After the subject has generated an immune response against the polypeptide, polyclonal antibodies can be obtained from the blood or serum of the subject that may selectively bind the polypeptide. If desired, B-cell lymphocytes can be obtained from the subject and tested using serial dilution to identify a B-cell lymphocyte that produces a monoclonal antibody that can selectively bind the polypeptide. Production of these monoclonal antibodies is typically based on the fusion of antibody generating spleen cells from immunized mice, rats, or rabbits with immortal myeloma cell lines. A variety of hybridoma and antibody engineering techniques can be used to produce polyclonal or monoclonal antibodies against the polypeptide, e.g., as reviewed in Saeed et al. (2017). In some embodiments, human antibodies can be generated in vitro by antibody engineering technologies such as phage display, construction of antibody fragments, immunomodulatory antibodies, and cell-free systems (Edwards and He, 2012). In some embodiments, the antibodies can be humanized via methods such as framework-homology-based humanization, germline humanization, complementary determining regions (CDR)-homology-based humanization and specificity determining residues (SDR) grafting (e.g., as described in Safdari et al., 2013).

As used herein, the term “polypeptide” encompasses amino acid chains comprising at least 50 amino acid residues, and more preferably at least 75 amino acid residues or at least 100 amino acid residues, wherein the amino acid residues are linked by covalent peptide bonds. As used herein, an “antigenic polypeptide” or an “immunoreactive polypeptide” is a polypeptide which, when introduced into a vertebrate, can stimulate the production of antibodies in the vertebrate, i.e., is antigenic, and wherein the antibody can selectively recognize and/or bind the antigenic polypeptide. An antigenic polypeptide may comprise or consist of an immunoreactive sequence derived from an immunoreactive Ehrlichia protein as described herein (e.g., as shown in Table 1, more preferably Table 3), and the polypeptide may comprise one or more additional sequences. In some embodiments, the additional sequences may be derived from a native Ehrlichia antigen and may be heterologous, and such sequences may (but need not) be immunogenic. In some embodiments, the antigenic polypeptide or immunoreactive polypeptide is covalently bound to a solid substrate, e.g., in an immunoassay such as a lateral flow test, etc.

Ehrlichia immunoreactive polypeptides as described herein may be a recombinant polypeptide, synthetic polypeptide, purified polypeptide, immobilized polypeptide, detectably labeled polypeptide, encapsulated polypeptide, or a vector-expressed polypeptide. In various embodiments, the Ehrlichia immunoreactive polypeptides provided herein may be truncated or may comprise a deletion mutation, without eliminating the immunoreactivity of the resulting peptide or polypeptide. An immunoreactive peptide or polypeptide disclosed herein may also be comprised in a pharmaceutical composition such as, e.g., a vaccine composition that is formulated for administration to a human or canine subject.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B: Immunoreactivity screening of E. ch. and E. ca. hypothetical proteins by ELISA. (FIG. 1A) An HME patient serum was used to screen E. ch. hypothetical proteins. (FIG. 1B) Pooled CME dog sera were used to screen E. ca. hypothetical proteins. OD values represent the mean optical density reading from 3 wells (±standard deviations) after background subtraction. A sample OD of ≥0.1 was considered positive and ≥0.5 a strong positive after subtracting negative control (IVTT reaction with the empty vector template) reading.

FIG. 2: Immunoreactivity of 15 hypothetical proteins of E. ch. and comparison with 3 TRPs by ELISA. The IVTT products reacted with a panel of sera from 10 HME patients. A normal human serum did not recognize these proteins. OD values represent the mean optical density reading from 3 wells (±standard deviations) after background subtraction. A sample OD of ≥0.1 was considered positive and ≥0.5 a strong positive after subtracting negative control (IVTT reaction with empty plasmid template) reading. The top 6 proteins with mean OD of ≥1.0 were considered immunodominant.

FIG. 3: Immunoreactivity of 16 E. ca. hypothetical proteins and comparison with TRP19 by ELISA. The IVTT proteins reacted with sera from 10 E. ca.-infected dogs. OD values represent the mean optical density reading from 3 wells (±standard deviations) after background subtraction. A sample OD of ≥0.1 was considered positive and ≥0.5 a strong positive after subtracting negative control (IVTT reaction with empty plasmid template) reading. The top 8 proteins with mean OD of ≥1.0 were considered immunodominant.

FIGS. 4A-B: Conformational immunoreactivity of 15 recombinant E. ch. hypothetical proteins. (FIG. 4A) Immunoreactivity comparison of the denaturing recombinant hypothetical proteins and TRPs detected by ELISA with a panel of sera from 10 HME patients. (FIG. 4B) Immunoreactivity of overlapping synthetic peptides spanning 3 immunoreactive proteins containing positive peptides, as determined by ELISA with an HME patient serum. Positive control, TRP120. OD values represent the mean optical density reading from 3 wells (±standard deviations) after background subtraction. A sample OD of ≥0.1 was considered positive and ≥0.5 a strong positive after subtracting negative control (A: IVTT reaction with empty plasmid template; B: a negative peptide) reading.

FIGS. 5A-B: Conformation immunoreactivity of 16 E. ca. hypothetical proteins. (FIG. 5A) Immunoreactivity of the denaturing E. ca. hypothetical proteins compared with TRP19 by ELISA. The IVTT proteins reacted with sera from 10 E. ca.-infected dogs. (FIG. 5B) Immunoreactivity of overlapping synthetic peptides spanning 3 E. ca. immunoreactive proteins containing positive peptides, as determined by ELISA with a dog serum. Positive control, TRP19. OD values represent the mean optical density reading from 3 wells (±standard deviations) after background subtraction. A sample OD of ≥0.1 was considered positive and ≥0.5 a strong positive after subtracting negative control (A: IVTT reaction with empty plasmid template; B: a negative peptide) reading.

FIG. 6: Expression of E. ch. and E. ca. hypothetical proteins by IVTT. Recombinant expression of randomly selected hypothetical proteins of E. ch. (upper) and E. ca. (bottom) by IVTT was detected by dot immunoblotting with anti-His-tag antibody. CTL, the negative control (IVTT reaction without plasmid template).

FIGS. 7A-B: Immunoreactivity screening of E. ch. and E. ca. hypothetical proteins by ELISA. (FIG. 7A) Pooled HME patient sera were used to screen E. ch. hypothetical proteins. (FIG. 7B) Pooled CME dog sera were used to screen E. ca. hypothetical proteins. ELISA OD values represent the mean optical density reading from 3 wells (±standard deviations) after background subtraction. A sample OD of ≥0.2 was considered positive after subtracting negative control (IVTT reaction with the empty vector template) reading.

FIG. 8: Immunoreactivity of 22 E. ch. proteins and TRP120 comparison by ELISA. IVTT-expressed proteins were probed with a panel of convalescent sera from 8 HME patients. A normal human serum control did not recognize these proteins. ELISA OD values represent the mean optical density reading from 3 wells (±standard deviations) after background subtraction. A sample OD of ≥0.2 was considered positive after subtracting negative control (IVTT reaction with empty plasmid template) reading. The top 12 proteins with mean OD of ≥1.0 were considered immunodominant.

FIG. 9: Immunoreactivity of 18 E. ca. proteins and TRP19 comparison by ELISA. IVTT-expressed proteins were probed with convalescent sera from 10 dogs with CME. OD values represent the mean optical density reading from 3 wells (±standard deviations) after background subtraction. A sample OD of ≥0.2 was considered positive after subtracting negative control (IVTT reaction with empty plasmid template) reading. The top 9 proteins with mean OD of ≥1.0 were considered immunodominant.

FIGS. 10A-B: Conformation-dependent immunoreactivity of 22 recombinant E. ch. proteins. (FIG. 10A) Immunoreactivity comparison of the denatured IVTT-expressed proteins and TRPs by ELISA using a panel of 8 sera from HME patients. (FIG. 10B) Immunoreactivity of overlapping synthetic peptides spanning 3 immunoreactive proteins, as determined by ELISA with an HME patient serum. Positive control, TRP120. OD values represent the mean optical density reading from 3 wells (±standard deviations) after background subtraction. A sample OD of ≥0.2 was considered positive after subtracting negative control (A: IVTT reaction with empty plasmid template; B: a negative peptide) reading.

FIG. 11A-B: Conformation-dependent immunoreactivity of 18 E. ca. hypothetical proteins. (FIG. 11A) Immunoreactivity of the IVTT-expressed denatured E. ca. proteins compared with TRP19 by ELISA. The IVTT proteins reacted with convalescent sera from 10 CME dogs. (FIG. 11B) Immunoreactivity of overlapping synthetic peptides spanning 3 E. ca. immunoreactive proteins, as determined by ELISA with a CME dog serum. Positive control, TRP19. ELISA OD values represent the mean optical density reading from 3 wells (±standard deviations) after background subtraction. A sample OD of ≥0.2 was considered positive after subtracting negative control (A: IVTT reaction with empty plasmid template; B: a negative peptide) reading.

FIG. 12: A schematic diagram of E. ch./E. ca. new antigen discovery showing our identification strategy and the different investigated proteins.

FIG. 13: Expression of E. ch. and E. ca. proteins by IVTT. Detection of IVTT expression of selected proteins of E. ch. (upper) and E. ca. (bottom) by dot immunoblot with anti-His-tag antibody. CTL, the negative control (IVTT reaction without plasmid template).

FIG. 14: Conformation-dependent immunoreactivity of E. ch. and E. ca. proteins. Immunoreactivity of the native and denatured proteins and TRPs was detected by dot immunoblot with serum from an HME patient or a CME dog. All proteins were IVTT-expressed and purified. CTL, the negative control (IVTT protein with empty plasmid template).

### DESCRIPTION OF ILLUSTRATIVE EMBODIMENTS

In some aspects, an immunoreactive polypeptide (e.g., in Table 1, more preferably Table 3, Table 4, or Table 5) described herein may be used in diagnostic or prophylactic tools for detection of or immunization against Ehrlichia infection. For example, the immunoreactive polypeptides may be used in solution-phase assays, or in assays in which the isolated immunoreactive polypeptide is immobilized on a surface of a support substrate. An immunoreactive polypeptide described herein or an RNA encoding a polypeptide described herein may be comprised in a vaccine formulation to induce an immune response, such as a protective immune response, in a subject against Ehrlichia chaffeensis or Ehrlichia canis. One or more immunoreactive polypeptides can be immobilized on a surface by covalent attachment, encapsulation, or adsorption using methods generally known in the art, and may include the use of cross-linkers, capture molecules and such like, to which peptides or polypeptides may be coupled, conjugated, or cross-linked. In some embodiments, an mRNA encoding a polypeptide of Table 3, Table 4, or Table 5 is included in a pharmaceutical composition; for example, the mRNA may be chemically modified and comprised in a viral vector (e.g., an adenovirus

As shown in the below examples, immunomolecular characterization of Ehrlichia chaffeensis (E. ch.) and E. canis (E. ca.) has defined protein orthologs, including tandem repeat proteins (TRPs) that have immunodominant linear antibody epitopes. Bioinformatic analysis and high-throughput protein expression and immunoscreening approaches were used to identify immunoreactive E. ch. and E. ca. hypothetical proteins. Antigenicity of the E. ch. and E. ca. ORFeomes (n=1105 and n=925, respectively) was analyzed by the sequence-based prediction model ANTIGENpro, and ˜250 ORFs were identified in each respective ORFeome as highly antigenic. The hypothetical proteins (E. ch. n=93 and E. ca. n=98) present in the top 250 antigenic ORFs were further investigated in this study. By ELISA, 46 E. ch. and 30 E. ca. IVTT-expressed hypothetical proteins reacted with antibodies in sera from naturally E. ch.-infected patients or E. ca.-infected dogs. Moreover, 15 E. ch. and 16 E. ca. proteins consistently reacted with a panel of sera from patients or dogs, including many that rivaled the immunoreactivity of “gold standard” TRPs. Antibody epitopes in most (˜70%) of these proteins exhibited partial or complete conformation-dependence. The majority (23/31; 74%) of the major novel immunoreactive proteins identified were small (≤250 aa), and 20/31 (65%) were predicted to be secreted effectors. Unlike the strong linear antibody epitopes previously identified in TRP and OMP orthologs, there were contrasting differences in the E. ch. and E. ca. antigenic repertoires, epitopes and ortholog immunoreactivity. These immunodominant and subdominant antigens may be used in diagnostic tests for Ehrlichia infection and/or in vaccine compositions against Ehrlichia chaffeensis (E. ch.) and/or E. canis (E. ca.). In particular, Ecaj_0126, Ecaj_0717, Ecaj_0636, Ecaj_0920, Ecaj_0259, and Ecaj_0922 continued to react with dog sera even after denaturation of the immunoreactive protein, supporting the idea that these immunoreactive proteins have linear epitopes. In contrast, after denaturation, Ecaj_0348, Ecaj_0748, Ecaj_0676, Ecaj_0723, Ecaj_0736, Ecaj_0730 and Ecaj_0767 exhibited markedly reduced or no reactivity with most dog sera tested, supporting the idea that these proteins have conformation-dependent antibody epitopes. Without wishing to be bound by any theory, the results also demonstrate that E. ch. and E. ca. have very different conformational immunomes that are largely not shared between the species.

Additionally, 216 E. ch. and 190 E. ca. highly antigenic proteins were further analyzed ANTIGENpro and also performed a genome-wide hypothetical protein analysis (E. ch. n=104; E. ca. n=124) for antibody immunoreactivity. Using cell-free protein expression and immunoanalysis, 118 E. ch. and 39 E. ca. proteins reacted with sera from naturally E. ch.-infected patients or E. ca.-infected dogs. Several of the hypothetical proteins were unexpectedly more reactive to sera than would have been expected based on the predicted antigenicity using ANTIGENpro. 22 E. ch. and 18 E. ca. proteins consistently and strongly reacted with a panel of patient or canine sera. Numerous E. ch. (n=12) and E. ca. (n=9) proteins were identified as immunodominant. Most of the immunoreactive proteins were classified as hypothetical and the antibody epitopes exhibited complete or partial conformation-dependence. The majority (28/40; 70%) of the E. ch and E. ca. proteins contained transmembrane domains and 19 (48%) were predicted to be secreted effectors. The antigenic repertoires of E. ch. and E. ca. were mostly diverse and suggest that the immunomes of these closely related ehrlichiae are dominated by species-specific conformational antibody epitopes.

### I. Immobilized Immunoreactive Polypeptides

In some embodiments, an immunoreactive polypeptide provided herein (e.g., Table 1, more preferably Table 3) is immobilized onto a surface of a support or a solid substrate; for example, the immunoreactive polypeptide may be immobilized directly or indirectly by coupling, cross-linking, adsorption, encapsulation, or by any appropriate method known in the art. By way of non-limiting example, binding of an immunoreactive polypeptide disclosed herein by adsorption to a well in a microtiter plate or to a membrane may be achieved by contacting the peptide, in a suitable buffer, with the well surface for a suitable amount of time. The contact time can vary with temperature, but is typically between about 1 hour and 1 day when using an amount of peptide ranging from about 50 ng to about 1 mg, and preferably about 250-700 ng or about 450-550 ng.

In some embodiments, an immunoreactive polypeptide disclosed herein is covalently attached to a support substrate by first reacting the support with a reagent that will chemically react with both the support and a functional group (i.e., crosslink), such as a hydroxyl or amino group, on the peptide. For example, an immunoreactive polypeptide may be crosslinked to a surface through an amine or carboxylic group on either end of the peptide, and a peptide may be crosslinked through a group on each end of the polypeptide (i.e., head-to-tail crosslinked). Such peptomers (i.e., head-to-tail crosslinked or otherwise immobilized peptides) may be used with both diagnostic and therapeutic methods of the present embodiments.

Numerous support substrates for polypeptide immobilization are known in the art which may be employed with an immunoreactive polypeptide disclosed herein, formed from materials such as, for example, latex, polystyrene, nylon, nitrocellulose, cellulose, silica, agarose, inorganic polymers, lipids, proteins, sugars, or magnetic resin. A person of ordinary skill in the art may select the support substrate that is appropriate for a given application. In particular embodiments of the present invention, a support substrate may be a reaction chamber, a microplate well, a membrane, a filter, a paper, an emulsion, a bead, a microbead, a microsphere, a nanocrystal, a nanosphere, a dipstick, a card, a glass slide, a microslide, a lateral flow apparatus, a microchip, a comb, a silica particle, a magnetic particle, a nanoparticle, or a self-assembling monolayer.

### II. Detectably-Labeled Immunoreactive Polypeptides

An immunoreactive polypeptide (e.g., in Table 1, more preferably Table 3) may be conjugated to or attached to detectable label such as, for example, a radioactive isotope, a non-radioactive isotope, a particulate label, a fluorescent label, a chemiluminescent label, a paramagnetic label, an enzyme label or a colorimetric label. The detectably-labelled polypeptide may be used, e.g., in diagnostic or prophylactic methods and compositions. In certain embodiments, the polypeptide portion of the detectably labeled immunoreactive polypeptide may be immobilized on a surface of a support substrate. In other embodiments, the detectable label may be used to immobilize the detectably labeled immunoreactive peptide to the surface of a support substrate.

As used herein, “detectable label” is a compound and/or element that can be detected due to its specific functional properties, and/or chemical characteristics, the use of which allows the peptide to which it is attached be detected, and/or further quantified if desired.

In some embodiments, the detectable label is a photoluminescent probe, such as a fluorophore or a nanoparticle, such as for example a strontium aluminate nanoparticle (e.g., see Paterson et al., 2014). Exemplary labels include, but are not limited to, a particulate label such as colloidal gold, a radioactive isotope such as astatine²¹¹, ¹⁴carbon, ₉₅chromium, ³⁶chlorine, ⁵⁷cobalt, ⁵⁸cobalt, copper⁶⁷, ¹⁵²Eu, gallium⁶⁷, ³hydrogen, iodine¹²³, iodine¹²⁵, iodine¹³¹, indium¹¹, ⁹iron, ³²phosphorus, rhenium186, rhenium188, ⁷⁵selenium, ³⁵sulphur, technicium-99, technetium-99m or yttrium⁹⁰, a colorimetric label such as dinitrobenzene, dansyl chloride, dabsyl chloride, any of the azo, cyanin or triazine dyes, or chromophores disclosed in U.S. Pat. Nos. 5,470,932, 5,543,504, or 6,372,445, all of which are incorporated herein by reference; a paramagnetic label such as chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) or erbium (III), a fluorescent label such as Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, or Lucifer Yellow, an enzyme label such as urease, luciferase, alkaline phosphatase, (horseradish) hydrogen peroxidase, or glucose oxidase, or a chemiluminescent label such as luminol, phthalazinedione, and others disclosed in any of U.S. Pat. Nos. 4,373,932, 4,220,450, 5,470,723, and U.S. Patent Application 2007/0264664, all of which are incorporated herein by reference.

### III. Methods of Producing an Immunoreactive Polypeptide

An immunoreactive polypeptide of the present embodiments may be produced using in vitro transcription and translation (IVTT) methods, may be recombinantly produced using a variety of cell types (e.g., bacterial cells, mammalian cells, E. coli, yeast, insect cells, etc.), or in some instances may be synthesized (e.g., using solid-phase synthesis). In some embodiments, IVTT and synthetic methods can provide certain advantages over recombinant approaches, since the resulting polypeptides can produce highly pure forms without contaminating bacterial or other proteins that might result in false positive reactions when utilizing recombinant proteins. Thus, IVTT and synthetic methods have an advantage of lacking many of the costly and laborious purification procedures often associated with recombinant methodologies.

A variety of IVTT approaches are known in the art and may be used in various embodiments. IVTT generally involves cell-free methods for production or synthesis of a protein from DNA. The cell-free system for protein production may use, e.g., E. coli extract, protozoan extracts, yeast extracts, human cell extract, wheat germ extract, mammalian extracts, extracts from cultured human cell lines, rabbit reticulocyte lysate, insect cell extract, or reconstituted and purified E. coli components. A variety of kits are commercially available including, e.g., RTS (FivePrime, San Francisco, Calif.), Expressway™ (Life Technologies); S30 T7 high yield (Promega), One-step human IVT (Thermo Scientific), WEPRO@ (CellFree Sciences), TNT® coupled (Promega), RTS CECF (5 PRIME), TNT® Coupled (Promega), Retic lysate IVT™ (Life Technologies); TNT® T7 (Promega), EasyXpress Insect kit (Qiagen/RiN A), PURExpress® (New England Biolabs), and PURESYSTEM® (BioComber). Such methods can be used to incorporate unnatural amino acids into proteins, if desired. Cell-free expression systems that may be used in various embodiments are also described, e.g., in Zemella et al., 2015.

An isolated immunoreactive protein as disclosed herein may be produced in some embodiments using an appropriate method known in the organic chemistry arts. For example, peptides may be produced using one of the established solid-phase peptide synthesis techniques, such as those of Merrifield, Carpino, or Atherton [Atherton and Sheppard, 1989]. In some embodiments, peptides may be synthesized using equipment for automated peptide synthesis that is widely available from commercial suppliers such as Perkin Elmer (Foster City, Calif.), or the peptide may be chemically synthesized using solution-phase techniques such as those described in Carpino et al., 2003 or U.S. Patent App. 2009/0005535. In some embodiments, the peptides or shorter proteins may be synthesized, e.g., using solid-phase peptide synthesis (SPPS), t-Boc solid-phase peptide synthesis, or Fmoc solid-phase peptide synthesis.

In some embodiments, an immunoreactive protein as described herein can be recombinantly prepared from a nucleic acid encoding the peptide. Such a nucleic acid may be operably linked to an expression vector. By way of nonlimiting example, an immunoreactive protein may be expressed from a vector and isolated from the growth media of a host cell comprising the vector. In some embodiments, the immunoreactive protein may be produced in a cell-free system from a nucleic acid encoding the peptide.

An immobilized immunoreactive protein as disclosed herein may be conjugated, crosslinked, or adsorbed, either directly or indirectly onto a surface of a support substrate. In some embodiments, an immobilized immunoreactive protein or peptide may be synthesized onto a support substrate.

It is anticipated that virtually any method of protein or peptide immobilization known in the art which would not impact the structure or function of the disclosed polypeptides may be used to immobilize an immunoreactive protein or polypeptide as disclosed herein. For example, peptide immobilization may be accomplished using a crosslinking or conjugation agent such as methyl-p-hydroxybenzimidate, N-succinimidyl-3-(4-hydroxyphenyl)propionate, using sulfosuccinimidyl 4-(N-maleimidomethyl)cyclohexane-1-carboxylate (sSMCC), N-[maleimidocaproyloxy]sulfosuccinimide ester (sEMCS), N-maleimidobenzoyl-N-hydroxysuccinimide ester (MBS), glutaraldehyde, 1-ethyl-3-(3-dimethylaminopropyl) carbodiimide (EDCI), Bis-diazobenzidine (BDB), or N-acetyl homocysteine thiolactone (NAHT), and others disclosed in any of U.S. Pat. Nos. 5,853,744, 5,891,506, 6,210,708, 6,617,142, 6,875,750, 6,951,765, 7,163,677, and 7,282,194, each incorporated herein by reference. Immunoreactive proteins may be conjugated directly or indirectly to any of the commercially available support substrates having a surface coatings comprising crosslinkers, coupling agents, thiol or hydroxyl derivatizing agents, carboxyl- or amine-reactive groups such as of maleic anhydride (e.g., Pierce Immunotechnology Catalog and Handbook, at A12-A13, 1991).

In some embodiments, a polypeptide as disclosed herein may also be immobilized using metal chelate complexation, employing, for example, an organic chelating agent such a diethylenetriaminepentaacetic acid anhydride (DTPA); EDTA; N-chloro-p-toluenesulfonamide; and/or tetrachloro-3α-6 α-diphenylglycouril-3 attached to the antibody (U.S. Pat. Nos. 4,472,509 and 4,938,948, each incorporated herein by reference). Proteins, polypeptides and peptides can also be immobilized by coupling to other peptides or to condensation groups immobilized on a surface or present in an immobilization buffer such as glutaraldehyde or periodate. Conjugates with fluorescence markers may also prepared in the presence of such agents or by reaction with an isothiocyanate. A peptide may be attached to a surface by conjugation, crosslinking or binding to an affinity binding agent such as biotin, streptavidin, a polysaccharide such as an alginate, a lectin, and the like.

In general, regardless of the method of preparation or immobilization status, the immunoreactive proteins disclosed herein are preferably prepared in a substantially pure form. Preferably, the immunoreactive proteins are at least about 80% pure, more preferably at least about 90% pure, even more preferably at least about 95% pure, and most preferably at least about 99% pure.

### IV. Ehrlichia Vaccine Compositions

Previous work has shown that Ehrlichial proteins that induce antibody responses can provide protective immune responses; thus, in some embodiments an immunoreactive protein provided herein (e.g., in Table 1, more preferably Table 3, Table 4, or Table 5) may be included in a pharmaceutical composition such as a vaccine composition for administration to a mammalian or human subject. For example, protection against E. chaffeensis infection has been demonstrated with epitope-specific antibodies directed at OMP and TRPs in in vitro models and in animal models (Kuriakose et al., 2012; Li et al., 2002; Li et al., 2001), demonstrating that ehrlichial proteins that elicit strong antibody responses to linear epitopes are protective.

The phrases “pharmaceutical,” “pharmaceutically acceptable,” or “pharmacologically acceptable” refers to molecular entities and compositions that do not produce an adverse, allergic or other untoward reaction when administered to an animal, such as, for example, a human, as appropriate. As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (e.g., Remington: The Science and Practice of Pharmacy; Pharmaceutical Press, 22nd Revised edition, 2012). Except insofar as any conventional carrier is incompatible with the active ingredient, its use in the vaccine compositions of the present invention is contemplated.

As used herein, a “protective immune response” refers to a response by the immune system of a mammalian host to an Ehrlichia antigen which results in increased recognition of the antigen and antibody production by the immune system of the mammalian host upon subsequent exposure to an Ehrlichia pathogen. A protective immune response may substantially reduce or prevent symptoms as a result of a subsequent exposure to Ehrlichia chaffeensis or Ehrlichia canis.

A person having ordinary skill in the medical arts will appreciate that the actual dosage amount of a vaccine composition administered to an animal or human patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

A. RNA Vaccines

In some aspects, a ribonucleic acid (RNA) comprising an open reading frame encoding a polypeptide of Table 1, more preferably Table 3, Table 4, or Table 5 is included in an RNA vaccine or pharmaceutical composition. The RNA is preferably a mRNA, and the RNA may be comprised in a lipid nanoparticle (e.g., comprising an ionizable cationic lipid, a neutral lipid, a sterol, and/or a PEG-modified lipid) or a viral vector (e.g., an adenovirus, an adeno-associated virus, etc.).

RNA vaccines offer a variety of advantages. Since mRNA is a non-infectious, non-integrating platform, there is no significant risk of infection or insertional mutagenesis. mRNA is degraded by normal cellular processes, and its in vivo half-life can be extended by chemical modifications and delivery methods (Kariko, et al., 2008; Kauffman, et al., 2016; Guan & Rosenecker, 2017; Thess et al., 2015; Kariko et al., 2011). Immunogenicity of mRNA can also be reduced, if desired, to further increase the safety profile (Kariko, et al., 2008; Thess et al., 2015; Kariko et al., 2011). Various modifications can make mRNA more stable and highly translatable (Kariko, et al., 2008; Thess et al., 2015; Kariko et al., 2011). Efficient in vivo delivery can be achieved by formulating mRNA into carrier molecules, which can allow for rapid uptake and expression in the cytoplasm (reviewed in Kauffman, et al., 2016; Guan & Rosenecker, 2017).

The RNA may be comprised in a variety of formulations, such as nanoparticles and lipid nanoparticles. In some embodiments the RNA or mRNA is comprised in protamine, a protamine liposome, polysaccharide particle, cationic nanoemulsion, cationic polymer, cationic polymer liposome, cationic lipid nanoparticle, nanoparticle comprising cationic lipid and cholesterol, nanoparticle comprising (cationic lipid, cholesterol, and PEG), or a dendrimer nanoparticle. These different formulations are further discussed, e.g., in Pardi et al., 2018.

The RNA or mRNA may be chemically modified or unmodified (also called naked RNA). A variety of modifications to the RNA or mRNA can be made, e.g., to extend the half-life of the RNA after injection to a mammalian subject, such as a dog or a human. Modifications to the mRNA that can be made include a 5′ cap, 5′- and 3′-UTRs, optimization of the coding region, and/or including the poly(A) tail; these modifications can be used, e.g., to improve intracellular stability and/or translational efficiency. In some embodiments, when a 5′ cap is not included on the mRNA, the nRNA may include an internal ribosome entry site (IRES) to promote function. Codon optimization can be included to improve translation or reduce endonucleolytic attack. a poly(A) tail may be included in the mRNA to promote stability. Modified nucleotides can be included to inhibit deadenylation. A 3′ UTR can be included to promote proper translation and intracellular trafficking. A 5′ UTR can be included, optionally with or without an IRES, to promote proper translation and intracellular trafficking and to reduce 5′-exonucleolytic degradation. 5′ caps include 5′-5′-triphosphate bridge (ppp) (m7GpppN structure) and anti-reverse cap analogues (ARCAs; m27,3′-OGpppG). 3′-UTR sequences include 3′-UTR of the eukaryotic elongation factor 1α (EEF1A1) mRNA. Codon optimization can be used to improve translation efficiency; for example, replacing rare codons with synonymous frequent codons improves translational yield. Nonetheless, in some embodiments, codon optimization is not used; for example, some proteins require slow translation, which is ensured by rare codons, for their proper folding. A variety of modifications are known and can be used in various embodiments (e.g., Sahin et al., 2014)

Lipid nanoparticles (LNPs) are used in some embodiments to deliver the RNA or mRNA. LNPs generally include four components: an ionizable cationic lipid, which may promote self-assembly into virus-sized (−100 nm) particles and help endosomal release of mRNA to the cytoplasm; lipid-linked polyethylene glycol (PEG), which may increase the half-life of formulations; cholesterol, a stabilizing agent; and naturally occurring phospholipids, which can support lipid bilayer structure. LNPs can be used for effective in vivo delivery of self-amplifying RNA and non-replicating mRNA. LNPs can be delivered via intradermal, intramuscular and subcutaneous administration have been shown to produce prolonged protein expression at the site of the injection. The magnitude and duration of in vivo protein production from mRNA-LNP vaccines can be affected by varying the route of administration. For example, intramuscular and intradermal delivery of mRNA-LNPs may result in more persistent protein expression than other systemic delivery routes.

In some embodiments, the lipid nanoparticle comprises 20-60% ionizable cationic lipid, 5-25% neutral lipid (e.g., disteroylphosphatidyl choline (DSPC)), 25-55% sterol (e.g., cholesterol), and 0.5-15% PEG-modified lipid (e.g., PEG-DMG or PEG-cDMA). Lipid nanoparticle formulations are also discussed in US2020/0197510 and U.S. Pat. No. 10,702,600 that can be used in various embodiments of the present disclosure. LNPs can optionally contain chitosan, cationic 1,2 dioleoyloxy 3 trimethylammoniumpro-pane (DOTAP), dioleoylphosphatidylethanolamine (DOPE), or ionizable dendrimer, if desired.

The RNA or mRNA may be included in a self-amplifying or replicon RNA vaccine or a non-replicating mRNA vaccine. In some preferred embodiments, the RNA or mRNA is included in a non-replicating mRNA vaccine. Self-amplifying mRNA (SAM) vaccines typically include portions of an alphavirus genome, wherein genes encoding the RNA replication machinery are included but the genes encoding the structural proteins are replaced with the antigen of interest (e.g., Perri et al., J. Virol. 77, 10394-10403 (2003)). Preferably, the mRNA is included in a directly injectable, non-replicating mRNA vaccine.

In some embodiments, the RNA or mRNA is produced via Good Manufacturing Practices (GMP) techniques. GMP production of mRNA typically begins with DNA template production followed by enzymatic IVT. Depending on the specific mRNA construct and chemistry, the protocol may be modified to accommodate modified nucleosides, capping strategies and/or template removal. To initiate the production process, template plasmid DNA (e.g., produced in Escherichia coli) can be linearized using a restriction enzyme to allow synthesis of runoff transcripts with a poly(A) tract at the 3′ end. Next, the mRNA can be synthesized from NTPs by a DNA-dependent RNA polymerase from bacteriophage (e.g., such as T7, SP6, or T3). The template DNA can then be degraded by incubation with DNase. The mRNA can then be enzymatically or chemically capped to enable efficient translation in vivo. mRNA synthesis can be very productive, e.g., yielding in excess of 2 gl⁻¹ of full-length mRNA in multi-gram scale reactions under optimized conditions. After synthesis of the mRNA, additional purification steps (e.g., microbeads in batch or column formats) can be performed to remove reaction components including enzymes, free nucleotides, and any residual DNA and/or truncated RNA fragments.

B. DNA Vaccines

In some embodiments, pharmaceutical composition comprising a DNA encoding a polypeptide of Table 1, more preferably Table 3, Table 4, or Table 5 is included in an DNA vaccine or pharmaceutical composition. DNA vaccines can be delivered via a viral vector, such as an adenovirus or adeno-associated virus (AAV). In some embodiments, RNA vaccines are preferable over DNA vaccines because RNA vaccines do not require a viral vector and may be less expensive to manufacture.

A variety of viral vectors can be used. For example, adenoviruses that can be used in various embodiments include those described in U.S. Pat. Nos. 9,714,435 and 9,701,718. Adenoviral vectors include AD26 and ChAdOx1 (derived from a chimpanzee adenovirus).

C. Peptide and Polypeptide Vaccines

In select embodiments, it is contemplated that an immunoreactive polypeptide of Table 1, more preferably Table 3, Table 4, or Table 5, can be comprised in a vaccine composition and administered to a subject (e.g., a human or dog) to induce an immune response in the subject against an Ehrlichia organism such as Ehrlichia chaffeensis or Ehrlichia canis. In some embodiments, the immune response is a protective immune response that that may substantially prevent or ameliorate infection in the subject by an the Ehrlichia organism. A vaccine composition for pharmaceutical use in a subject may comprise an immunoreactive polypeptide of Table 1, 2, or 3 and a pharmaceutically acceptable carrier.

In some embodiments, a vaccine composition of the present invention may comprise an immunoreactive polypeptide (e.g., having a sequence that has at least about 90%, 91%, 92%, 93%, 94%, 95%, 96%, 97%, 98%, 99%, or 100% sequence identity to a polypeptide listed of Table 1 or more preferably Table 3, Table 4, or Table 5). In some embodiments, a vaccine composition comprising the immunoreactive polypeptide may be used to induce an immune response, such as a protective immune response, against Ehrlichia chaffeensis or Ehrlichia canis (e.g., in a human or dog subject).

In certain embodiments, vaccine compositions may comprise, for example, at least about 0.1% of an immunoreactive polypeptide (e.g., of Table 1, more preferably Table 3). In other embodiments, the immunoreactive polypeptide may comprise between about 2% to about 75% of the weight of the unit, or between about 25% to about 60%, for example, and any range derivable therein. As with many vaccine compositions, frequency of administration, as well as dosage, will vary among members of a population of animals or humans in ways that are predictable by one skilled in the art of immunology. By way of nonlimiting example, the pharmaceutical compositions and vaccines may be administered by injection (e.g., intracutaneous, intramuscular, intravenous or subcutaneous), intranasally (e.g., by aspiration) or orally. Between 1 and 3 doses may be administered over a 1-36 week period. Preferably, 3 doses are administered (e.g., at intervals of 3-4 months), and booster vaccinations may be given periodically thereafter.

In some embodiments, a “suitable dose” is an amount of an immunoreactive polypeptide that, when administered as described above, can result in an immune response in an immunized patient sufficient to reduce the symptoms of or provide some protection against a subsequent exposure to an Ehrlichia organism. In general, the amount of peptide present in a suitable dose (or produced in situ by the nucleic acid in a dose) may range from about 1 pg to about 500 mg per kg of host, typically from about 10 pg to about 10 mg, preferably from about 100 pg to about 1 mg and more preferably from about 100 pg to about 100 microgram.

A vaccine composition of the present invention may utilize a variety of different types of carriers depending on whether it is to be administered in solid, liquid or aerosol form, and whether it needs to be sterile for such routes of administration as injection. A vaccine composition disclosed herein can be administered intramuscularly, intradermally, subcutaneously, intravenously, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subconjunctivally, intravesicularly, mucosally, intrapericardially, locally, orally, intranasally, or by inhalation, injection, infusion, continuous infusion, lavage, or localized perfusion. A vaccine composition may also be administered to a subject via a catheter, in cremes, in lipid compositions, by ballistic particulate delivery, or by other method or any combination of the forgoing as would be known to one of ordinary skill in the art (see, for example, Remington: The Science and Practice of Pharmacy, 21^{st} Ed. Lippincott Williams and Wilkins, 2005, incorporated herein by reference).

While any suitable carrier known to those of ordinary skill in the art may be employed in the vaccine compositions of this invention, the type of carrier will vary depending on the mode of administration. For parenteral administration, such as subcutaneous injection, the carrier preferably comprises water, saline, alcohol, a fat, a wax or a buffer. For oral administration, any of the above carriers or a solid carrier, such as mannitol, lactose, starch, magnesium stearate, sodium saccharine, talcum, cellulose, glucose, sucrose, and magnesium carbonate, may be employed. Biodegradable microspheres (e.g., polylactic galactide) may also be employed as carriers for the pharmaceutical compositions of this invention. Suitable biodegradable microspheres are disclosed, for example, in U.S. Pat. Nos. 4,897,268 and 5,075,109.

In some embodiments, vaccine composition can be administered by microstructured transdermal or ballistic particulate delivery. Microstructures as carriers for vaccine formulation are a desirable configuration for vaccine applications and are widely known in the art (e.g., U.S. Pat. Nos. 5,797,898, 5,770,219 and 5,783,208, and U.S. Patent Application 2005/0065463). Such a vaccine composition formulated for ballistic particulate delivery may comprise an isolated immunoreactive polypeptide of Table 1, more preferably Table 3, immobilized on a surface of a support substrate. In these embodiments, a support substrate can include, but is not limited to, a microcapsule, a microparticle, a microsphere, a nanocapsule, a nanoparticle, a nanosphere, or a combination thereof.

Microstructures or ballistic particles that serve as a support substrate for an immunoreactive polypeptide disclosed herein may contain a biodegradable material or non-biodegradable material, and such support substrates may be comprised of synthetic polymers, silica, lipids, carbohydrates, proteins, lectins, ionic agents, crosslinkers, and other microstructure components available in the art. Protocols and reagents for the immobilization of a peptide of the invention to a support substrate composed of such materials are widely available commercially and in the art.

In other embodiments, a vaccine composition comprises an immobilized or encapsulated immunoreactive polypeptide (e.g., of Table 1, more preferably Table 3, Table 4, or Table 5) and a support substrate. In these embodiments, a support substrate can include, but is not limited to, a lipid microsphere, a lipid nanoparticle, an ethosome, a liposome, a niosome, a phospholipid, a sphingosome, a surfactant, a transferosome, an emulsion, or a combination thereof. The formation and use of liposomes and other lipid nano- and microcarrier formulations is generally known to those of ordinary skill in the art, and the use of liposomes, microparticles, nanocapsules and the like have gained widespread use in delivery of therapeutics (e.g., U.S. Pat. No. 5,741,516, specifically incorporated herein in its entirety by reference). Numerous methods of liposome and liposome-like preparations as potential drug carriers, including encapsulation of peptides, have been reviewed (U.S. Pat. Nos. 5,567,434; 5,552,157; 5,565,213; 5,738,868 and 5,795,587, each of which is specifically incorporated in its entirety by reference).

In addition to the methods of delivery described herein, a number of alternative techniques are also contemplated for administering the disclosed vaccine compositions. By way of nonlimiting example, a vaccine composition may be administered by sonophoresis (i.e., ultrasound) which has been used and described in U.S. Pat. No. 5,656,016 for enhancing the rate and efficacy of drug permeation into and through the circulatory system; intraosseous injection (U.S. Pat. No. 5,779,708), or feedback-controlled delivery (U.S. Pat. No. 5,697,899), and each of the patents in this paragraph is specifically incorporated herein in its entirety by reference.

A polypeptide may be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids such as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

Sterile injectable solutions are prepared by incorporating the active peptides in the required amount in the appropriate solvent with various of the other ingredients enumerated above, as required, followed by filtered sterilization. Generally, dispersions are prepared by incorporating the various sterilized active ingredients into a sterile vehicle that contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. In some embodiments, prolonged absorption of an injectable composition can be brought about by the use in the compositions of agents delaying absorption, such as, for example, aluminum monostearate, gelatin or combinations thereof.

D. Adjuvants

In some aspects, an immunogenic composition comprising one or more chimeric polypeptide as disclosed herein (e.g., a polypeptide of Table 1, more preferably Table 3, Table 4, or Table 5) also contains an adjuvant. In some embodiments, the composition is a pharmaceutical preparation or a vaccine composition. A variety of adjuvants are known that can be included. For example, adjuvants such as MF59, AS01, AS02, AS03, AS04, Virosomes, CAF01, CAF04, CAF05, Montanide ISA™ 720, or Montanide ISA™ 51 (e.g., Bonam et al., 2017) can be used in some embodiments.

Any of a variety of adjuvants may be employed in the vaccines of this invention to nonspecifically enhance the immune response. Most adjuvants contain a substance designed to protect the antigen from rapid catabolism, such as aluminum hydroxide or mineral oil, and a nonspecific stimulator of immune responses, such as lipid A, Bortadella pertussis or Mycobacterium tuberculosis. Suitable adjuvants are commercially available as, for example, Freund's Incomplete Adjuvant and Freund's Complete Adjuvant (Difco Laboratories, Detroit, Mich.) and Merck Adjuvant 65 (Merck and Company, Inc., Rahway, N.J.). Other suitable adjuvants include alum, biodegradable microspheres, monophosphoryl lipid A and quil A.

In some embodiments, the vaccine composition further comprises an Ehrlichia canis bacterin or Ehrlichia chaffeensis bacterin. Methods that may be used to generate the bacterin include, but are not limited to, treatment of the Ehrlichia with heat, formaldehyde, formalin, bi-ethylene amine, radiation, or beta-propiolactone treatment. It is anticipated that an E. chaffeensis or E. canis bacterin may be inactivated by any suitable method available, such as, e.g., those described in WO2005087803, EP2433646, Vega et al., 2007; or Stuen et al., 2015.

In some embodiments, the immunogenic or vaccine composition includes an adjuvant comprising a triterpenoid, sterol, immunomodulator, polymer, and/or Th2 stimulator. For example, in some embodiments the adjuvant comprises DEAE Dextran, an immunostimulatory oligonucleotide, and oil (e.g., a light mineral oil), wherein the immunostimulatory oligonucleotide is a CpG containing ODN, and wherein the adjuvant formulation is a water-in-oil (W/O) emulsion. The vaccine adjuvant may optionally comprise an Ehrlichia bacterin (such as a heat-inactivated E. Canis or E. chaffeensis) and/or a chimeric peptide as disclosed herein (e.g., of Formula I or Table 2). In some embodiments, the immunogenic or vaccine composition includes an antigen component and an adjuvant formulation comprising a saponin (e.g., present in an amount of about 1 pg to about 5,000 pg per dose), a sterol (e.g., present in an amount of about 1 pg to about 5,000 pg per dose), a quaternary ammonium compound (e.g., present in an amount of about 1 pg to about 5,000 .mu.g per dose), a polymer (e.g., present in an amount of about 0.0001% v/v to about 75% v/v.), and an ORN/ODN; the saponin may be Quil A or a purified faction thereof, the sterol may be cholesterol, the quaternary ammonium compound may be dimethyl dioctadecyl ammonium bromide (DDA), the polymer may be polyacrylic acid, and the ORN/ODN may be a CpG. The adjuvant may comprise a glycolipid, such N-(2-deoxy-2-L-leucylamino-β-D-glucopyranosyl)-N-octadecyldodecanamide acetate. The adjuvant may comprise an immunostimulatory oligonucleotide, a polyacrylic acid polymer and at least two of the following: (a) dimethyl dioctadecyl ammonium bromide (DDA); (b) a sterol; and/or (c)N-(2-deoxy-2-L-leucylamino-β-D-glucopyranosyl)-N-octadecyldodecanamide acetate. For example, the vaccine composition may comprise an adjuvant as described, e.g., in U.S. Pat. 10,238,736, U.S. Pat. No. 8,580,280, or US Publication 2019/0008953.

In some embodiments, immunogenic or vaccine composition includes an antigen component and an adjuvant formulation comprising a triterpenoid saponin, a sterol, a quaternary ammonium compound, and a polyacrylic acid polymer, wherein the antigen component comprises or consists of a Ehrlichia bacterin (such as a heat-inactivated E. Canis) and/or a chimeric polypeptide as disclosed herein (e.g., a polypeptide of Formula I or of Table 2). In some embodiments, the saponin is present in an amount of about 1 mg to about 5,000 mg per dose, the sterol is present in an amount of about 1 mg to about 5,000 mg per dose, the quaternary ammonium compound is present in an amount of about 1 mg to about 5,000 mg per dose, and the polyacrylic acid polymer is present in an amount of about 0.0001% v/v to about 75% v/v. For example, the vaccine composition may comprise an adjuvant as described, e.g., in U.S. Pat. No. 9,662,385.

In some aspects, an immunogenic or vaccine composition as disclosed herein comprises an oil-based adjuvant comprising an Ehrlichia bacterin (such as a heat-inactivated E. Canis or E. chaffensis) and/or one or more chimeric polypeptide as disclosed herein (e.g., a polypeptide of Formula I or of Table 2). For example, the adjuvant formulation may comprise an oily phase and an aqueous phase, a polycationic carrier (e.g., DEAE dextran), and a CpG containing immunostimulatory oligonucleotide, wherein the vaccine is a water-in-oil emulsion. The adjuvant may optionally further comprise an aluminum hydroxide gel. In some embodiments, the CpG containing immunostimulatory oligonucleotide is present in the amount of about 50 to about 400 pg per dose and DEAE Dextran is present in the amount of about 10 to about 300 mg per dose. The adjuvant formulation may comprise an immunostimulating oligonucleotide, polycationic carrier, sterol, saponin, quaternary amine, TLR-3 agonist, glycolipid, and/or MPL-A (or an analog thereof) in an oil emulsion. For example, the vaccine composition may comprise an adjuvant as described, e.g., in U.S. Pat. No. 10,117,921 or US 2019/0038737.

In some embodiments, the immunogenic composition is an emulsion comprising (i) an Ehrlichia bacterin (such as a heat-inactivated E. Canis or E. chaffeensis), and/or (ii) one or more chimeric polypeptide as disclosed herein (e.g., a polypeptide of Formula I or of Table 2). For example, the emulsion composition may comprise an adjuvant, such as acrylic polymer and/or dimethyl dioctadecyl ammonium bromide (DDA), in the aqueous phase. The emulsion can be prepared, in some embodiments, by mixing an aqueous phase containing the antigen (e.g., an E. Canis bacterin such as a heat-inactivated E. Canis, and/or one or more chimeric polypeptide (e.g., a polypeptide of Formula I or of Table 2)) and adjuvant with an oil phase in the presence of an emulsifier. In some embodiments, the adjuvant component comprises an oil-in-water emulsion, wherein the aqueous phase of the oil-in-water emulsion comprises dimethyl dioctadecyl ammonium bromide (DDA) and/or an alkyl-polyacrylic acid (alkyl-PAA). In some embodiments, the oil in the oil-in-water emulsion is mineral oil, a terpene oil, soybean oil, olive oil, or a propylene glycol derivative. The adjuvant may further comprise the adjuvant component further comprises CpG DNA, a lipopolysaccharide, and/or monophosphoryl lipid A. The vaccine may further comprise one or more emulsifiers. For example, the vaccine composition may comprise an adjuvant as described, e.g., in U.S. Pat. No. 9,545,439 or U.S. Pat. No. 8,980,288.

In various embodiments, adjuvants such as MF59 (e.g., Calabro et al. (2013) Vaccine 31: 3363-3369), AS01 (Didierlaurent, et al. (2014) J. Immunol. 193, 1920-1930), AS02 (Garçon and Van Mechelen (2011) Expert Rev. Vaccines 10, 471-486), AS03 (Morel, S. et al. (2011) Vaccine 29, 2461-2473), AS04 (Didierlaurent, et al. (2009) J. Immunol. 183: 6186-6197.), Virosomes (Künzi, et al. (2009) Vaccine 27, 3561-3567), CAF01 (Tandrup Schmidt, et al. (2016) Pharmaceutics 8, 7.), CAF04 (Billeskov, et al. (2016) PLoS One 11, e0161217), CAF05 (Billeskov, et al. (2016) PLoS One 11, e0161217), Montanide ISA™ 720 (Aucouturier, et al. (2002) Expert Rev. Vaccines 1, 111-118), or Montanide ISA™ 51 (Aucouturier, et al. (2002) Expert Rev. Vaccines 1, 111-118) can be used. Table 6 provides a listing of example adjuvant containing formulations that can be used in various embodiments.

TABLE 6
Example adjuvant containing formulations

| Adjuvant | Composition |
|---|---|
| MF59 | Squalene, Span 85, Tween 80, and citrate buffer |
| AS01 | Liposomes containing 3-O-desacyl-4’-monophosphoryl lipid A |
| | (MPLA) and QS21 |
| AS02 | Oil-in-water (O/W) emulsion containing MPLA and the saponin QS21 |
| AS03 | α-tocopherol, squalene, polysorbate 80, and PBS |
| AS04 | Contains MPLA adsorbed onto a particulate form of aluminum salt |
| Virosomes | Contain inactivated virus |
| CAF01 | Cationic liposomal vehicle containing dimethyl dioctadecyl- |
| | ammonium (DDA) with a glycolipid immunostimulator (TDB) |
| CAF04 | Cationic liposomal vehicle containing DDA with monomycoloyl |
| | glycerol analog (MMG) |
| CAF05 | Cationic liposomal vehicle containing DDA with the |
| | immunostimulators TDB and poly(I:C) |
| Montanide ISA ™ 720 | Water-in-oil (W/O) emulsion containing non-mineral oil with |
| | mannide mono-oleate family emulsifier |
| Montanide ISA ™ 51 | W/O emulsion containing mineral oil with mannide |
| | mono-oleate family emulsifier |
| Acrylic polymer/ | Oil-in-water emulsion comprises dimethyl dioctadecyl ammonium |
| DDA emulsions | bromide (DDA) and/or an alkyl-polyacrylic acid (alkyl-PAA); e.g., see |
| | U.S. Pat. No. 9,545,439 or U.S. Pat. No. 8,980,288. |
| CpG/DEAE emulsions | Emulsions comprising a polycationic carrier (e.g., DEAE dextran) and |
| | a CpG containing immunostimulatory oligonucleotide; e.g., see |
| | U.S. Pat. No. 10,117,921 or US 2019/0038737. |
| Saponin/cholesterol/ | Saponin (e.g., Quil A), cholesterol, DDA, a polyacrylic acid; e.g., a |
| DDA adjuvants | triterpenoid saponin, a sterol, a quaternary ammonium compound, and |
| | a polyacrylic acid polymer; e.g., see U.S. Pat. No. 9,662,385. |
| Polyacrylic acid | Water-in-oil (W/O) emulsions, DEAE Dextran, immunostimulatory |
| polymer emulsions | oligonucleotide (e.g., a CpG containing ODN), a sterol, N-(2-deoxy-2- |
| | L-leucylamino-β-D-glucopyranosyl)-N-octadecyldodecanamide |
| | acetate, and/or a polyacrylic acid polymer; e.g., see U.S. Pat. No. |
| | 10,238,736, U.S. Pat. No. 8,580,280, or US Publication 2019/0008953. |

### V. Biological Functional Equivalents

Preferred immunoreactive polypeptides or analogs thereof specifically or preferentially bind an Ehrlichia chaffeensis or Ehrlichia canis specific antibody. Determining whether or to what degree a particular immunoreactive polypeptide, or an analog thereof, can bind an E canis specific antibody can be assessed using an in vitro assay such as, for example, an enzyme-linked immunosorbent assay (ELISA), immunoblotting, immunoprecipitation, radioimmunoassay (RIA), immunostaining, latex agglutination, indirect hemagglutination assay (IHA), complement fixation, indirect immnunofluorescent assay (FA), nephelometry, flow cytometry assay, chemiluminescence assay, lateral flow immunoassay, u-capture assay, mass spectrometry assay, particle-based assay, inhibition assay and/or an avidity assay.

An immunoreactive polypeptide of the present embodiments may be modified to contain amino acid substitutions, insertions and/or deletions that do not alter their respective interactions with anti-Ehrlichia antibody binding regions. Such a biologically functional equivalent of an immunoreactive polypeptide derived from an Ehrlichia protein could be a molecule having like or otherwise desirable characteristics, i.e., binding of Ehrlichia specific antibodies. As a nonlimiting example, certain amino acids may be substituted for other amino acids in an immunoreactive polypeptide disclosed herein without appreciable loss of interactive capacity, as demonstrated by detectably unchanged antibody binding. It is thus contemplated that an immunoreactive polypeptide disclosed herein (or a nucleic acid encoding such a polypeptide) which is modified in sequence and/or structure, but which is unchanged in biological utility or activity, remains within the scope of the present embodiments. The immunoreactive polypeptide may have, e.g., at least 90%, at least 95%, or at least 99% sequence identity with a polypeptide of Table 1 or Table 3, and in some embodiments the immunoreactive protein may have 1, 2, 3, 4, 5, or more amino acid substitutions, insertions and/or deletions as compared to a polypeptide of Table 1 or Table 3.

It is also well understood by the skilled artisan that, inherent in the definition of a biologically functional equivalent peptide, is the concept that there is a limit to the number of changes that may be made within a defined portion of the molecule while still maintaining an acceptable level of equivalent biological activity. Biologically functional equivalent polypeptides are thus defined herein as those peptides in which certain, not most or all, of the amino acids may be substituted. Of course, a plurality of distinct peptides with different substitutions may easily be made and used in accordance with the invention.

The skilled artisan is also aware that where certain residues are shown to be particularly important to the biological or structural properties of a peptide (e.g., residues within an epitope) such residues may not generally be exchanged. It is anticipated that a mutation in an epitope of an immunoreactive peptide or polypeptide disclosed herein could result in a loss of species-specificity and in turn, reduce the utility of the resulting peptide for use in methods of the present embodiments. Thus, polypeptides that are antigenic (i.e., bind anti-Ehrlichia antibodies specifically) and comprise conservative amino acid substitutions are understood to be included in the present embodiments. Conservative substitutions are least likely to drastically alter the activity of a protein. A “conservative amino acid substitution” refers to replacement of amino acid with a chemically similar amino acid, i.e., replacing nonpolar amino acids with other nonpolar amino acids; substitution of polar amino acids with other polar amino acids, acidic residues with other acidic amino acids, etc.

Amino acid substitutions, such as those which might be employed in modifying an immunoreactive polypeptide disclosed herein are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

Isoforms of the immunoreactive polypeptides disclosed herein can be used in some embodiments. An isoform contains the same number and kinds of amino acids as an immunoreactive polypeptide as disclosed herein, but the isoform has a different molecular structure. The isoforms contemplated by the present embodiments are those having the same properties as a polypeptide as described herein.

Nonstandard amino acids may be incorporated into proteins by chemical modification of existing amino acids or by de novo synthesis of a polypeptide disclosed herein. A nonstandard amino acid refers to an amino acid that differs in chemical structure from the twenty standard amino acids encoded by the genetic code, and a variety of nonstandard amino acids are well known in the art.

In select embodiments, the present disclosure contemplates a chemical derivative of an immunoreactive polypeptide disclosed herein. “Chemical derivative” refers to a peptide having one or more residues chemically derivatized by reaction of a functional side group, and retaining biological activity and utility. Such derivatized polypeptides include, for example, those in which free amino groups have been derivatized to form specific salts or derivatized by alkylation and/or acylation, p-toluene sulfonyl groups, carbobenzoxy groups, t-butylocycarbonyl groups, chloroacetyl groups, formyl or acetyl groups among others. Free carboxyl groups may be derivatized to form organic or inorganic salts, methyl and ethyl esters or other types of esters or hydrazides and preferably amides (primary or secondary). Chemical derivatives may include polypeptides that comprise one or more naturally occurring amino acids derivatives of the twenty standard amino acids. For example, 4-hydroxyproline may be substituted for serine; and ornithine may be substituted for lysine.

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues. The amino acids described herein are preferred to be in the “L” isomeric form. However, residues in the “D” isomeric form can be substituted for any L-amino acid residue, as long as the desired functional properties set forth herein are retained by the protein. In keeping with standard protein nomenclature, abbreviations for amino acid residues are known in the art.

In addition to the biological functional equivalents discussed above, it is contemplated that structurally similar compounds may be formulated to mimic the key portions of an immunoreactive peptide disclosed herein. Such compounds, which may be termed peptidomimetics, may be used in the same manner as immunoreactive peptides disclosed herein and, hence, also are functional equivalents. Methods for generating specific structures are disclosed, e.g., in Mizuno et al., 2017, as well as in U.S. Pat. Nos. 5,446,128; 5,710,245; 5,840,833; 5,859,184; 5,440,013; 5,618,914; and 5,670,155.

### VI. Methods of Detecting Ehrlichia Infection

Ehrlichiosis in humans generally refers to infections caused by obligate intracellular bacteria in the family Anaplasmataceae, chiefly in the genera Ehrlichia and Anaplasma. The majority of cases of human ehrlichiosis (HE) are caused by 3 distinct species: Ehrlichia chaffeensis, chief among them (Dumler et al., 2007). Ehrlichia infections in animals are also referred to as ehrlichiosis, along with a variety of diseases caused by a diverse group of pathogens from genuses Ehrlichia, Anaplasma, Neorickettsia, and Cowdria (Dumler et al., 2007). Ehrlichia infections are sustained mostly in monocytes or granulocytes, and studies have demonstrated that antibodies play an essential role in the immune response to Ehrlichia infection (Feng and Walker, 2004; Winslow et al., 2003; Winslow et al., 2000; Yager et al., 2005).

Accordingly, select embodiments of the present disclosure provide methods of detecting antibodies that specifically bind an Ehrlichia organism in a sample. Such a method may involve contacting a polypeptide of Table 1, more preferably Table 3, with the test sample, under conditions that allow peptide-antibody complexes to form, and detecting the peptide-antibody complexes. In these embodiments, the detection of the peptide-antibody complexes is an indication that antibodies specific for an Ehrlichia organism are present in the test sample, and the absence of the peptide-antibody complexes is an indication that antibodies specific an Ehrlichia organism are not present in the test sample.

In some embodiments, detection of an immunoreactive polypeptide disclosed herein bound to an Ehrlichia specific antibody (i.e., a peptide-antibody complex) can be accomplished using an enzyme-linked immunoassay (e.g., a sandwich ELISA, or a competitive ELISA), a radioimmunoassay, an immunoprecipitation, a fluorescence immunoassay, a chemiluminescent assay, an immunoblot assay, a lateral flow assay, a flow cytometry assay, a mass spectrometry assay, latex agglutination, an indirect hemagglutination assay (IHA), complement fixation, an inhibition assay, an avidity assay, a dipstick test, or a particulate-based assay. In some preferred embodiments, peptide-antibody complexes described herein are detected using an enzyme-linked immunoassay, a lateral flow assay, or a particle-based assay.

As used herein, a “sample” is any sample that comprises or is suspected to comprise antibodies. Preferably, the sample is whole blood, sputum, serum, plasma, saliva, cerebrospinal fluid or urine. In some embodiments, the sample is a blood, serum or plasma sample obtained from a subject or patient.

Ehrlichiosis caused by an Ehrlichia canis infection in humans presents with flu-like symptoms of fever, chills, headache, and muscle aches. In more severe cases, nausea, loss of appetite, weight loss, abdominal pain, cough, diarrhea and change in mental status may also be observed. Ehrlichiosis in humans is potentially fatal.

In dogs, ehrlichiosis is most often caused by either Ehrlichia chaffeensis or Ehrlichia canis bacteria, and progresses in three phases: an acute phase, a subclinical phase, and a chronic phase. The acute phase normally extends weeks after infection and features symptoms similar to those of human ehrlichiosis, such as fever, lethargy, loss of appetite, shortness of breath, joint pain and stiffness, and may also include more severe symptoms such as anemia, depression, bruising, and enlarged lymph nodes, liver, and spleen. The subclinical phase can persist for years and most often presents no symptoms, although antibodies to Ehrlichia antigens may be detectable. The chronic phase of Ehrlichia infection generally features recurring symptoms of weight loss, anemia, neurological dysfunction, bleeding, ocular inflammation, leg edema, and fever, and presents a blood profile which often leads to a misdiagnosis of leukemia. An Ehrlichia infection that progresses to the chronic stage of disease is often fatal.

The nonspecific symptoms of an Ehrlichia infection and their resemblance to mild and severe influenza symptoms makes diagnosis of Ehrlichiosis difficult in humans and dogs. Diagnosis can be further hampered by current laboratory testing procedures for Ehrlichia infection which are not point-of-care tests, i.e., the tests are not available in most hospitals, clinics, and physician or veterinarian offices where a patient can receive treatment.

Accordingly, select embodiments of the present invention provide methods of identifying an Ehrlichia infection in a mammalian subject. Such a method may involve contacting a sample from the subject with an isolated immunoreactive polypeptide disclosed herein (e.g., from Table 1, more preferably Table 3, Table 4, or Table 5) under conditions that allow peptide-antibody complexes to form, and detecting the peptide-antibody complexes. In these embodiments, the detection of the peptide-antibody complexes is an indication that the subject has an Ehrlichia infection. The Ehrlichia organism may be an Ehrlichia chaffeensis organism or an Ehrlichia canis organism. In some embodiments, the subject is a human or a dog. As with other methods disclosed herein, the detection step may be accomplished using any appropriate type of assay known in the art, and may be preferrably accomplished using a lateral flow assay or an ELISA.

The terms “subject” and “patient” are used interchangeably herein, and may refer to a mammal, especially a human or a dog. In certain embodiments, a “subject” or “patient” refers to a mammalian Ehrlichia host (i.e., animal infected with an Ehrlichia organism). An Ehrlichia host may be, for example, human or non-human primate, bovine, canine, caprine, cavine, corvine, epine, equine, feline, hircine, lapine, leporine, lupine, murine, ovine, porcine, racine, vulpine, and the like, including livestock, zoological specimens, exotics, as well as companion animals, pets, and any animal under the care of a veterinary practitioner. A subject may be or may not be infected with an Ehrlichia organism, and a subject may be a mammal suspected of being infected with an Ehrlichia organism.

Without wishing to be bound by theory, the ehrlichial immunoreactive polypeptides disclosed herein each comprise at least a part of a major Ehrlichia epitope that accounts for a species-specific immunogenicity in humans and animals. The term “epitope” is used herein to indicate that portion of an immunogenic substance that is specifically identified, recognized, and bound by, an antibody or cell-surface receptor of a host immune system that has mounted an immune response to the immunogenic substance as determined by any method known in the art. (see, for example, Geysen et al., 1984). Thus, an epitope that is “species-specific” is an epitope that can be used to differentiate one species of the Ehrlichia genus from another Ehrlichia species.

Particular embodiments relate to determining whether a subject has been immunized against Ehrlichia or is actively infected with an Ehrlichia organism. In these embodiments, the method comprises contacting a sample from the subject with at least one isolated immunoreactive polypeptide (e.g., of Table 1, more preferably Table 3) that is not a component of an Ehrlichia vaccine, and detecting whether an antibody in the sample specifically binds to the isolated ehrlichial immunoreactive polypeptide. According to the method, if an antibody in the sample specifically binds to the isolated ehrlichial immunoreactive polypeptide, then this result indicates the subject has or has had an active Ehrlichia infection, and if an antibody does not specifically bind to the isolated ehrlichial immunoreactive peptide, then the subject has either been previously immunized with an Ehrlichia vaccine or is not infected with an Ehrlichia organism. The Ehrlichia organism may be an E. chaffeensis organism or an E. canis organism.

An immunoreactive polypeptide of Table 1, more preferably Table 3, may be used to bind an Ehrlichia-specific or E. chaffeensis-specific antibody using a variety of methods or kits. The specific binding between an antibody and an Ehrlichial polypeptide as disclosed herein may therefore be assessed by any appropriate method known in the art including, but not limited to, an enzyme-linked immunosorbent assay (ELISA), a sandwich ELISA, a competitive ELISA, immunoblotting, immunoprecipitation, radioimmunoassay (RIA), immunostaining, latex agglutination, indirect hemagglutination assay (IHA), complement fixation, indirect immnunofluorescent assay (FA), nephelometry, flow cytometry assay, chemiluminescence assay, lateral flow immunoassay, u-capture assay, mass spectrometry assay, particle-based assay, inhibition assay and avidity assay. Exemplary methods of detecting the binding of an Ehrlichia-specific antibody to an ehrlichial immunoreactive polypeptide as disclosed herein may include, for example, an ELISA performed in a microplate, a lateral flow test performed using a dipstick or lateral flow device, or a particulate-based suspension array assay, e.g., performed using the Bio-Plex® system (Bio-Rad Laboratories, Hercules, Calif., USA).

A. ELISA

In certain embodiments, the detection of a peptide-antibody complex described herein is accomplished using an enzyme linked immunosorbent assay (ELISA). This assay may be performed by first contacting an immunoreactive polypeptide (e.g., in Table 1, Table 2, Table 3, Table 4, Table 5, Ech_0991, Ecaj_0126, Ecaj_0717, Ecaj_0636, Ecaj_0920, Ecaj_0259, or Ecaj_0348) that has been immobilized on a solid support, commonly the well of a microtiter plate, with the sample, such that antibodies specific for the peptide within the sample are allowed to bind to the immobilized peptide. Unbound sample is then removed from the immobilized peptide and a detection reagent capable of binding to the immobilized antibody-polypeptide complex is added. The amount of detection reagent that remains bound to the solid support is then determined using a method appropriate for the specific detection reagent.

In some embodiments, the detection reagent contains a binding agent (such as, for example, Protein A, Protein G, immunoglobulin, lectin, or free antigen) conjugated or covalently attached to a reporter group or label. Exemplary reporter groups or labels include enzymes (e.g., horseradish peroxidase), substrates, cofactors, inhibitors, dyes, radionuclides, luminescent groups, fluorescent groups, and biotin. The conjugation of binding agent to reporter group or label may be achieved using standard methods known to those of ordinary skill in the art. Common binding agents may also be purchased conjugated to a variety of reporter groups from many commercial sources (e.g., Zymed Laboratories, San Francisco, Calif.; and Pierce, Rockford, Ill.).

In some embodiments, the presence or absence of Ehrlichia specific antibodies can be determined in the sample by comparing the level of a signal detected from a reporter group or label in the sample with the level of a signal that corresponds to a control sample or predetermined cut-off value. In certain embodiments, the cut-off value is based on or reflects the average mean signal obtained when the immobilized ehrlichial immunoreactive peptide is incubated with samples from an uninfected subject. The cut-off value may be determined using a statistical method or computer program.

B. Lateral Flow Tests

Lateral flow tests may also be referred to as immunochromatographic strip (ICS) tests or simply strip-tests. In general, a lateral flow test is a form of assay in which the test sample flows laterally along a solid substrate via capillary action, or alternatively, under fluidic control. Such tests are often inexpensive, require a very small amount (e.g., one drop) of sample, and can typically be performed reproducibly with minimal training. The economical simplicity and robustness of many lateral flow assay formats makes these types of tests ideal for identifying an Ehrlichia (e.g., E. canis) infection at the point of care, which can be particularly important when the subject is, for example, a human or dog exhibiting detectable antibodies during the treatable acute phase of infection.

Exemplary lateral flow device formats include, but are not limited to, a dipstick, a card, a chip, a microslide, and a cassette, and it is widely demonstrated in the art that the choice of format is largely dependent upon the features of a particular assay. Accordingly, lateral flow devices are now ubiquitous in human and veterinarian medicine and quite varied, providing many options to the ordinarily skilled artisan for detecting a peptide-antibody complex in a sample using a lateral flow assay (e.g., any of U.S. Pat. Nos. 7,344,893, 7,371,582, 6,136,610, and U.S. Patent Applications, 2005/0250141 and 2005/0047972, or Koczula et al., 2016). By way of a nonlimiting example, a sample from a subject suspected of having an Ehrlichia infection is applied to a lateral flow device comprising at least a sample zone and a binding zone. The sample may be a serum sample or blood sample, and may be drawn laterally from the sample zone to the binding zone which comprises an immunoreactive polypeptide disclosed herein (e.g., of Table 1, Table 3, Ecaj_0919, Ecaj_0073, Ecaj_0104, or Ecaj_0663) immobilized to a surface of the lateral flow device. In this example, the binding of the immobilized ehrlichial immunoreactive polypeptide on the lateral flow device (e.g., by an antibody or antibodies from a blood or serum sample from the subject) is an indication that Ehrlichia specific antibodies are present in the sample from the subject, indicating an Ehrlichia infection in the subject, such as an E. chaffeensis or E. canis infection in the subject.

In related embodiments, an ELISA assay as described above may be performed in a rapid flow-through, lateral flow, or strip test format, wherein the antigen is immobilized on a membrane, such as a nitrocellulose membrane. In this flow-through test, Ehrlichia antibodies within the sample bind to the immobilized ehrlichial immunoreactive peptide as the sample passes through the membrane. A detection reagent, such as protein A labeled with gold, a fluorophore, or a chromophore, binds to the peptide-antibody complex as the solution containing the detection reagent flows through the membrane. Peptide-antibody complexes bound to detection reagent may then be detected, as appropriate for the detection reagent used (e.g., based on the presence or absence of a visibly detectable color or fluorescent label, a nanoparticle, a luminescent rare earth nanoparticle, a luminous nanoparticle, a strontium aluminate nanoparticle (e.g., see Paterson et al., 2014; and Wang et al., 2017, etc.). In some embodiments, detection of binding of an antibody from a biological sample from the subject with the immunogenic protein can be observed, e.g., by the binding of a labeled anti-human antibody or a labeled anti-dog antibody to the antibody bound to the immunoreactive polypeptide.

In some embodiments, a flow-through format ELISA may be performed in which one end of the membrane to which an immunoreactive peptide (e.g., from Table 1, more preferably Table 3) is immobilized may be immersed in a solution containing the sample, or the sample may be added to an area (i.e., a sample zone) at one end of the membrane. The sample may migrate along the membrane through a region (i.e., a labeling zone) comprising the detection reagent, and flows to the area (i.e., a binding zone) comprising the immobilized ehrlichial immunoreactive peptide. An accumulation of detection reagent at the binding zone indicates the presence of Ehrlichia specific antibodies in the sample.

Typically, a flow-through ELISA may feature a detection reagent applied to a test strip in a pattern, such as a line, that can be read visually. As with other lateral flow tests, the absence of such a pattern typically indicates a negative result. It is within the ability of an ordinarily skilled artisan to select an amount of the immunoreactive polypeptide for immobilization on the membrane that can generate a visually discernible pattern when the biological sample contains a level of antibodies that would be sufficient to generate a positive signal in a standard format ELISA. Preferably, the amount of peptide immobilized on the membrane ranges from about 25 ng to about 1 mg.

C. Particulate-Based Assays

In general, particle-based assays use a capture-binding partner, such as an antibody or an antigen in the case of an immunoassay, coated on the surface of particles, such as microbeads, crystals, chips, or nanoparticles. Particle-based assays may be effectively multi-plexed or modified to assay numerous variables of interest by incorporating fluorescently labeled particles or particles of different sizes in a single assay, each coated or conjugated to one or more labeled capture-binding partners. The use of sensitive detection and amplification technologies with particle-based assay platforms known in the art has resulted in numerous flexible and sensitive assay systems to choose from in performing a method described herein. For example, a multiplex particle-based assay such as the suspension array Bio-Plex® assay system available from Bio-Rad Laboratories, Inc. (Hercules, Calif.) and Luminex, Inc. (Austin, Tex.) may be useful in identifying Ehrlichia antibodies in a sample.

In an aspect, the present invention contemplates the immobilization of an isolated immunoreactive polypeptide (e.g., of Table 1, more preferably Table 3, Table 4, or Table 5) on a surface of a particle for use in a particle-based immunoassay. As described herein, methods of peptide immobilization onto support surfaces is well known in the art. In a preferred embodiment, a labeled her immunoreactive polypeptide disclosed herein is immobilized onto a surface of a particle and the peptide-particle complex is employed in an ELISA or in a flow cytometry assay according to established protocols.

### VII. Ehrlichia Detection and Vaccination Kits

Various embodiments of the present invention are concerned with kits for the detection of antibodies in a sample that specifically bind an Ehrlichia organism, such as E. chaffeensis or E. canis. The kits may thus be used for the diagnosis or identification of an Ehrlichia infection in a subject. In other embodiments, the invention provides kits for determining whether a subject has been immunized against Ehrlichia or is actively infected with an Ehrlichia organism. In still other embodiments, kits are provided for vaccination of a subject against Ehrlichia chaffeensis infection, and in some embodiments it is anticipated that the composition may be used to provide a protective immune response against an Ehrlichia canis infection.

In select embodiments, a kit of the present invention may be used to perform a method disclosed herein. For example, a kit may be suitable for detecting Ehrlichia antibodies in a sample, for identifying an Ehrlichia infection individual, for determining whether a subject has been immunized against Ehrlichia or is actively infected with an Ehrlichia organism, or for vaccinating a subject against an Ehrlichia organism. In these embodiments, one or more immunoreactive peptide (e.g., from Table 1, 2, or 3, or a polypeptide having at least about 95% or more sequence identity with a polypeptide of Table 1, more preferably Table 3 or a polypeptide having at least about 95% or more sequence identity with Table 1 or Table 3) may be comprised in the kit. The ehrlichial immunoreactive polypeptide in the kit may be detectably labeled or immobilized on a surface of a support substrate also comprised in the kit. The immunoreactive polypeptide(s) may, for example, be provided in the kit in a suitable form, such as sterile, lyophilized, or both.

The support substrate comprised in a kit of the invention may be selected based on the method to be performed. By way of nonlimiting example, a support substrate may be a multi-well plate or microplate, a membrane, a filter, a paper, an emulsion, a bead, a microbead, a microsphere, a nanobead, a nanosphere, a nanoparticle, an ethosome, a liposome, a niosome, a transferosome, a dipstick, a card, a celluloid strip, a glass slide, a microslide, a biosensor, a lateral flow apparatus, a microchip, a comb, a silica particle, a magnetic particle, or a self-assembling monolayer.

As appropriate to the method being performed, a kit may further comprise one or more apparatuses for delivery of a composition to a subject or for otherwise handling a composition of the invention. By way of nonlimiting example, a kit may include an apparatus that is a syringe, an eye dropper, a ballistic particle applicator (e.g., applicators disclosed in U.S. Pat. Nos. 5,797,898, 5,770,219 and 5,783,208, and U.S. Patent Application 2005/0065463), a scoopula, a microslide cover, a test strip holder or cover, and such like.

A detection reagent for labeling a component of the kit may optionally be comprised in a kit for performing a method of the present invention. In particular embodiments, the labeling or detection reagent is selected from a group comprising reagents used commonly in the art and including, without limitation, radioactive elements, enzymes, molecules which absorb light in the UV range, and fluorophores such as fluorescein, rhodamine, auramine, Texas Red, AMCA blue and Lucifer Yellow. In other embodiments, a kit is provided comprising one or more container means and a BST protein agent already labeled with a detection reagent selected from a group comprising a radioactive element, an enzyme, a molecule which absorbs light in the UV range, and a fluorophore.

In particular embodiments, the present invention provides a kit for detecting anti-Ehrlichia antibodies in a sample which may also be used for identification of an Ehrlichia infection in a subject, and/or for determining whether a subject has been immunized against Ehrlichia or is actively infected with an Ehrlichia organism. Such a kit may comprise one or more immunoreactive polypeptides (e.g., from Table 1, 2, or 3, or having at least about 95% or more sequence identity with a polypeptide of Table 1, 2, or 3; Ecaj_0919, Ecaj_0073, Ecaj_0104, or Ecaj_0663), and the peptides may be detectably labeled and immobilized to one or more support substrates comprised in the kit.

In some embodiments, a kit comprises an immunoreactive polypeptide of Table 1, 2, or 3 or having about 95% or more sequence identity with polypeptide of Table 1, 2, or 3. The peptides may be immobilized to one or more separate lateral flow assay devices, such as a nitrocellulose test strips. In these embodiments, each of the test strips may further comprises a detection reagent, for example, a chromophore-labeled protein A. Such a kit may further comprise one or more containers for sample material, one or more diluents for sample dilution, and one or more control indicator strips for comparison.

When reagents and/or components comprising a kit are provided in a lyophilized form (lyophilisate) or as a dry powder, the lyophilisate or powder can be reconstituted by the addition of a suitable solvent. In particular embodiments, the solvent may be a sterile, pharmaceutically acceptable buffer and/or other diluent. It is envisioned that such a solvent may also be provided as part of a kit.

When the components of a kit are provided in one and/or more liquid solutions, the liquid solution may be, by way of non-limiting example, a sterile, aqueous solution. The compositions may also be formulated into an administrative composition. In this case, the container means may itself be a syringe, pipette, topical applicator or the like, from which the formulation may be applied to an affected area of the body, injected into a subject, and/or applied to or mixed with the other components of the kit.

### IV. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Identification and Validation of Immunoreactive Proteins

Predicting E. ch. and E. ca. ORF antigenicity with ANTIGENpro

Antigenicity of 1105 E. ch. and 925 E. ca. ORFs (excluding RNA genes and pseudogenes) were predicted by ANTIGENpro. The antigenicity score of E. ch. and E. ca. ORFs ranged from 0.01 to 0.97, with the top 250 ORFs in the respective genomes scoring above a minimum threshold (−0.7) that has been shown to provide a balance of sensitivity, specificity and accuracy in predicting protein antigenicity (Magnan et al., 2010). Well-characterized major immunoreactive proteins of E. ch. and E. ca., including TRPs, Ank200, Omp1/OmpA (P28/P30) and Msp4 family members, were represented in the ANTIGENpro top 250, indicating concordance between previous experimental data and ANTIGENpro prediction (Table X1). Among the top 250 E. ch. and E. ca. ORFs, 93 (37%) and 98 (39%) ORFs were annotated as hypothetical without any putative function assigned in IMG database. The TRPs and Ank200, previously annotated as hypothetical, were excluded from this group. Based on existing empirical TRP/Ank data and other studies suggesting hypothetical proteins are frequent targets of the host immune response, the inventors focused on this group in this investigation. The E. ch. and E. ca. hypothetical proteins in the top 250 were ranked according to the ANTIGENpro score (from high to low) (Tables S1 and S2).

TABLE X1
ANTIGENpro antigenicity score and overall rank of
known E. ch. and E. ca. major immunoreactive proteins.

| | | | | Antigenicity |
|---|---|---|---|---|
| Ehrlichia | Protein | Tag no. | Rank | score |
| E. ch. | TRP47 | 0166 | 30 | 0.908 |
| | TRP32 | 0170 | 49 | 0.881 |
| | TRP120 | 0039 | 100 | 0.838 |
| | TRP75 | 0558 | 130 | 0.802 |
| | Ank200 | 0684 | 184 | 0.759 |
| | Omp1/P28 family | multiple^{a} | 68~233 | 0.861~0.711 |
| E. ca. | TRP36 | 0109 | 8 | 0.943 |
| | TRP19 | 0113 | 43 | 0.906 |
| | OmpA/P30 family | 0563,0918 | 49, 211 | 0.902, 0.737 |
| | Msp4 family | multiple^{b} | 113~236 | 0.835~0.714 |
| | TRP140 | 0017 | 227 | 0.724 |
| | Ank200 | 0365 | 237 | 0.714 |

^{a}Tag numbers include 1121, 1125, 1126, 1127, 1130, 1131, 1133, 1134, 1137, 1140, 1142 and 1144.
^{b}Tag numbers include 0831, 0833, 0896, 0905, 0906, 0911, 0913, 0914, 0915, 0916 and 0917.

TABLE S1
Highly antigenic E. ch. hypothetical proteins (n = 93) by ANTIGENpro (score ≥ 0.695).

| | | Antigenicity | Mean | Size | E. ca. ortholog |
|---|---|---|---|---|---|
| No. | Ech_tag no. | score | ELISA OD | (AA) | (Ecaj_tag no.) |
| 1 | 0187 | 0.969 | 0.06 | 563 | 0126 |
| 2 | 1147 | 0.964 | 0.05 | 126 | * |
| 3 | 0247 | 0.958 | 0.05 | 302 | * |
| 4 | 0261 | 0.956 | 1.08 | 264 | 0762 |
| 5 | 0255 | 0.950 | 0.89 | 338 | 0764 |
| 6 | 0253 | 0.950 | 0.01 | 189 | * |
| 7 | 0865 | 0.949 | 0.04 | 302 | 0229 |
| 8 | 1152 | 0.949 | 0.04 | 185 | 0923 |
| 9 | 0722 | 0.945 | 0.41 | 190 | * |
| 10 | 0246 | 0.944 | 0.03 | 275 | * |
| 11 | 0257 | 0.943 | 0.02 | 226 | * |
| 12 | 0609 | 0.935 | 0.01 | 301 | * |
| 13 | 0601 | 0.929 | 0.00 | 374 | 0434 |
| 14 | 0535 | 0.928 | 0.20 | 186 | 0500 |
| 15 | 0251 | 0.928 | 0.41 | 205 | * |
| 16 | 0576 | 0.924 | 0.04 | 98 | 0462 |
| 17 | 0150 | 0.923 | 0.05 | 672 | 0099 |
| 18 | 1037 | 0.920 | 0.02 | 1231 | 0835 |
| 19 | 0745 | 0.920 | 0.21 | 118 | 0324 |
| 20 | 0864 | 0.918 | 0.13 | 330 | 0231 |
| 21 | 0825 | 0.917 | 0.52 | 380 | 0259 |
| 22 | 0113 | 0.909 | 0.03 | 793 | * |
| 23 | 0166 | 0.908 | 0.50 | 285 | 0109 |
| 24 | 0862 | 0.907 | 0.00 | 403 | 0232 |
| 25 | 0531 | 0.905 | 0.42 | 175 | * |
| 26 | 0285 | 0.895 | 0.20 | 181 | * |
| 27 | 0744 | 0.889 | 0.06 | 157 | 0325 |
| 28 | 0612 | 0.888 | 0.00 | 208 | * |
| 29 | 0879 | 0.885 | 0.03 | 815 | * |
| 30 | 0147 | 0.885 | 0.47 | 193 | 0096 |
| 31 | 0611 | 0.880 | 0.19 | 229 | 0428 |
| 32 | 1036 | 0.880 | 0.04 | 750 | 0834 |
| 33 | 0525 | 0.879 | 0.01 | 666 | 0508 |
| 34 | 0252 | 0.875 | 0.61 | 364 | * |
| 35 | 0118 | 0.873 | 0.00 | 30 | * |
| 36 | 0807 | 0.864 | 0.05 | 334 | 0271 |
| 37 | 0348 | 0.862 | 0.12 | 202 | 0660 |
| 38 | 0763 | 0.860 | 0.33 | 165 | 0312 |
| 39 | 0106 | 0.858 | 0.00 | 713 | 0066 |
| 40 | 1154 | 0.857 | 0.14 | 135 | 0926 |
| 41 | 0120 | 0.857 | 0.00 | 213 | * |
| 42 | 0240 | 0.857 | 0.31 | 158 | * |
| 43 | 1148 | 0.854 | 0.21 | 142 | 0920 |
| 44 | 0243 | 0.853 | 0.02 | 293 | * |
| 45 | 0284 | 0.852 | 0.03 | 1016 | 0716 |
| 46 | 0115 | 0.851 | 0.00 | 203 | * |
| 47 | 0345 | 0.850 | 0.36 | 294 | 0663 |
| 48 | 0878 | 0.847 | 0.00 | 409 | * |
| 49 | 1021 | 0.845 | 0.02 | 219 | 0824 |
| 50 | 0700 | 0.845 | 0.15 | 192 | * |
| 51 | 0607 | 0.844 | 0.31 | 322 | 0434 |
| 52 | 0377 | 0.843 | 0.02 | 104 | 0636 |
| 53 | 0549 | 0.842 | 0.15 | 195 | * |
| 54 | 0614 | 0.839 | 0.54 | 231 | 0423 |
| 55 | 1103 | 0.830 | 0.26 | 223 | 0881 |
| 56 | 0846 | 0.828 | 0.48 | 171 | 0242 |
| 57 | 0199 | 0.823 | 0.00 | 213 | 0136 |
| 58 | 0108 | 0.819 | 0.01 | 825 | 0072/0071 |
| 59 | 0551 | 0.811 | 0.17 | 191 | 0479 |
| 60 | 1027 | 0.804 | 0.00 | 34 | * |
| 61 | 0663 | 0.802 | 0.14 | 202 | 0379 |
| 62 | 0578 | 0.798 | 0.37 | 185 | * |
| 63 | 0716 | 0.790 | 0.64 | 367 | 0347 |
| 64 | 0778 | 0.786 | 0.15 | 1132 | 0297 |
| 65 | 1013 | 0.785 | 0.06 | 203 | 0818 |
| 66 | 0398 | 0.781 | 0.41 | 121 | 0621 |
| 67 | 0991 | 0.779 | 0.70 | 710 | 0139 |
| 68 | 0927 | 0.775 | 0.02 | 34 | * |
| 69 | 0949 | 0.773 | 0.21 | 31 | * |
| 70 | 0259 | 0.773 | 0.36 | 118 | * |
| 71 | 0704 | 0.771 | 0.35 | 248 | 0351 |
| 72 | 0256 | 0.770 | 0.01 | 72 | * |
| 73 | 0181 | 0.769 | 0.15 | 103 | 0122 |
| 74 | 0297 | 0.769 | 0.07 | 272 | 0706 |
| 75 | 0388 | 0.768 | 0.71 | 293 | * |
| 76 | 0159 | 0.767 | 0.50 | 507 | 0104 |
| 77 | 1053 | 0.763 | 0.61 | 193 | 0846 |
| 78 | 0122 | 0.758 | 0.10 | 126 | 0073 |
| 79 | 0593 | 0.758 | 0.10 | 382 | 0445 |
| 80 | 0698 | 0.758 | 0.01 | 200 | * |
| 81 | 0079 | 0.756 | 0.01 | 134 | 0047 |
| 82 | 0986 | 0.752 | 0.16 | 179 | 0142 |
| 83 | 0715 | 0.748 | 0.14 | 551 | 0348 |
| 84 | 0279 | 0.747 | 0.01 | 41 | * |
| 85 | 0836 | 0.737 | 0.13 | 1201 | 0253 |
| 86 | 0281 | 0.716 | 0.39 | 179 | * |
| 87 | 0276 | 0.716 | 0.06 | 184 | * |
| 88 | 0526 | 0.715 | 0.07 | 495 | 0507 |
| 89 | 0478 | 0.704 | 0.29 | 172 | 0548 |
| 90 | 0126 | 0.704 | 0.04 | 334 | 0077 |
| 91 | 0866 | 0.703 | 0.06 | 330 | 0228 |
| 92 | 0945 | 0.699 | 0.00 | 1349 | 0174 |
| 93 | 0767 | 0.695 | 0.02 | 621 | 0309 |

* E.ca. ortholog not identified.

TABLE S2
Highly antigenic E. ca. hypothetical proteins (n = 98) by ANTIGENpro (score ≥ 0.710).

| | | Antigenicity | Mean | Size | E.ch. ortholog |
|---|---|---|---|---|---|
| No. | Ecaj_tag no. | score | ELISA OD | (AA) | (Ech_tag no.) |
| 1 | 0126 | 0.962 | 2.23 | 671 | 0187 |
| 2 | 0341 | 0.951 | 0.16 | 190 | * |
| 3 | 0920 | 0.947 | 1.44 | 182 | 1148 |
| 4 | 0715 | 0.945 | 0.02 | 1918 | * |
| 5 | 0099 | 0.945 | 0.02 | 630 | 0150 |
| 6 | 0923 | 0.942 | 0.27 | 184 | 1152 |
| 7 | 0063 | 0.940 | 0.10 | 705 | * |
| 8 | 0762 | 0.940 | 0.01 | 353 | 0261 |
| 9 | 0503 | 0.939 | 0.19 | 110 | * |
| 10 | 0069 | 0.938 | 0.04 | 823 | * |
| 11 | 0838 | 0.932 | 0.03 | 1510 | 1038 |
| 12 | 0431 | 0.929 | 0.03 | 436 | * |
| 13 | 0259 | 0.928 | 1.75 | 368 | 0825 |
| 14 | 0346 | 0.926 | 0.01 | 518 | * |
| 15 | 0071 | 0.925 | 0.51 | 641 | 0121 |
| 16 | 0500 | 0.925 | 0.00 | 185 | 0535 |
| 17 | 0716 | 0.925 | 0.10 | 1601 | 0284 |
| 18 | 0324 | 0.922 | 0.00 | 117 | 0745 |
| 19 | 0922 | 0.920 | 1.56 | 133 | * |
| 20 | 0067 | 0.919 | 0.00 | 695 | * |
| 21 | 0764 | 0.919 | 0.01 | 191 | 0255 |
| 22 | 0660 | 0.917 | 0.05 | 197 | 0348 |
| 23 | 0139 | 0.915 | 0.07 | 794 | 0991 |
| 24 | 0220 | 0.915 | 0.22 | 461 | * |
| 25 | 0230 | 0.915 | 0.04 | 486 | * |
| 26 | 0772 | 0.914 | 0.10 | 163 | * |
| 27 | 0924 | 0.912 | 0.06 | 144 | 1152 |
| 28 | 0066 | 0.912 | 0.02 | 889 | 0106 |
| 29 | 0835 | 0.911 | 0.02 | 1267 | 1037 |
| 30 | 0325 | 0.906 | 0.00 | 157 | 0744 |
| 31 | 0068 | 0.905 | 0.01 | 616 | * |
| 32 | 0881 | 0.904 | 0.00 | 305 | 1103 |
| 33 | 0462 | 0.903 | 0.00 | 93 | 0576 |
| 34 | 0228 | 0.899 | 0.09 | 345 | 0866 |
| 35 | 0072 | 0.899 | 0.03 | 916 | 0121 |
| 36 | 0312 | 0.897 | 0.00 | 188 | 0763 |
| 37 | 0345 | 0.897 | 0.05 | 631 | * |
| 38 | 0356 | 0.891 | 0.00 | 85 | * |
| 39 | 0824 | 0.890 | 0.24 | 242 | 1021 |
| 40 | 0073 | 0.887 | 1.70 | 92 | 0122 |
| 41 | 0379 | 0.886 | 0.10 | 182 | 0663 |
| 42 | 0528 | 0.885 | 0.00 | 199 | 0500 |
| 43 | 0743 | 0.885 | 0.00 | 84 | * |
| 44 | 0271 | 0.884 | 1.17 | 329 | 0807 |
| 45 | 0434 | 0.881 | 0.00 | 223 | 0601 |
| 46 | 0834 | 0.876 | 0.08 | 782 | 1036 |
| 47 | 0767 | 0.875 | 0.79 | 91 | * |
| 48 | 0065 | 0.871 | 0.08 | 595 | * |
| 49 | 0342 | 0.871 | 0.51 | 217 | * |
| 50 | 0232 | 0.868 | 0.16 | 412 | 0862 |
| 51 | 0062 | 0.867 | 0.04 | 957 | * |
| 52 | 0096 | 0.864 | 0.03 | 194 | 0147 |
| 53 | 0620 | 0.861 | 0.00 | 97 | 0399 |
| 54 | 0429 | 0.861 | 0.00 | 251 | * |
| 55 | 0771 | 0.852 | 0.06 | 245 | * |
| 56 | 0231 | 0.849 | 0.09 | 328 | 0864 |
| 57 | 0508 | 0.843 | 0.18 | 621 | 0525 |
| 58 | 0919 | 0.841 | 2.35 | 120 | 1147 |
| 59 | 0343 | 0.841 | 0.14 | 181 | * |
| 60 | 0450 | 0.840 | 0.02 | 330 | * |
| 61 | 0229 | 0.838 | 0.10 | 354 | 0865 |
| 62 | 0309 | 0.837 | 0.06 | 840 | 0767 |
| 63 | 0726 | 0.835 | 0.11 | 214 | * |
| 64 | 0730 | 0.831 | 0.67 | 165 | * |
| 65 | 0741 | 0.830 | 0.19 | 114 | * |
| 66 | 0428 | 0.829 | 0.07 | 290 | 0611 |
| 67 | 0625 | 0.821 | 0.02 | 112 | 0391 |
| 68 | 0926 | 0.820 | 0.06 | 135 | 1154 |
| 69 | 0185 | 0.819 | 0.17 | 271 | 0929 |
| 70 | 0663 | 0.818 | 0.02 | 293 | 0345 |
| 71 | 0736 | 0.814 | 0.60 | 188 | * |
| 72 | 0445 | 0.812 | 0.07 | 382 | 0593 |
| 73 | 0297 | 0.807 | 0.01 | 1130 | 0778 |
| 74 | 0717 | 0.804 | 2.06 | 226 | * |
| 75 | 0423 | 0.801 | 0.10 | 257 | * |
| 76 | 0047 | 0.801 | 0.02 | 133 | 0079 |
| 77 | 0748 | 0.797 | 1.75 | 121 | * |
| 78 | 0186 | 0.794 | 0.10 | 285 | 0929 |
| 79 | 0381 | 0.788 | 0.05 | 184 | 0660 |
| 80 | 0493 | 0.782 | 0.06 | 239 | 0540 |
| 81 | 0239 | 0.778 | 0.14 | 951 | * |
| 82 | 0142 | 0.776 | 0.03 | 178 | 0986 |
| 83 | 0348 | 0.767 | 1.35 | 535 | * |
| 84 | 0430 | 0.761 | 0.07 | 183 | * |
| 85 | 0676 | 0.760 | 0.65 | 229 | 0329 |
| 86 | 0636 | 0.759 | 1.70 | 98 | 0377 |
| 87 | 0122 | 0.756 | 0.03 | 103 | 0181 |
| 88 | 0347 | 0.756 | 0.78 | 354 | 0716 |
| 89 | 0482 | 0.756 | 0.02 | 92 | * |
| 90 | 0254 | 0.755 | 0.03 | 269 | 0835 |
| 91 | 0739 | 0.748 | 0.02 | 220 | * |
| 92 | 0253 | 0.739 | 0.03 | 1206 | 0836 |
| 93 | 0174 | 0.729 | 0.04 | 1306 | 0945 |
| 94 | 0725 | 0.728 | 0.03 | 190 | * |
| 95 | 0723 | 0.728 | 0.46 | 248 | * |
| 96 | 0727 | 0.714 | 0.02 | 97 | * |
| 97 | 0119 | 0.712 | 0.20 | 881 | 0176 |
| 98 | 0198 | 0.710 | 0.12 | 381 | 0907 |

* E.ch. ortholog not identified.

Expression, Immunoscreening and Identification of E. ch. And E. ca. Hypothetical Proteins

An in vitro transcription and translation (IVTT) system was used to express the E. ch. and E. ca. hypothetical ORFs. To confirm IVTT expression, dot blots were performed using anti-His-tag antibody on randomly selected proteins (17 from E. ch. and 18 from E. ca.). The expression of all proteins was detectable (FIG. 6) and the negative control (IVTT reaction without plasmid) was not detectable.

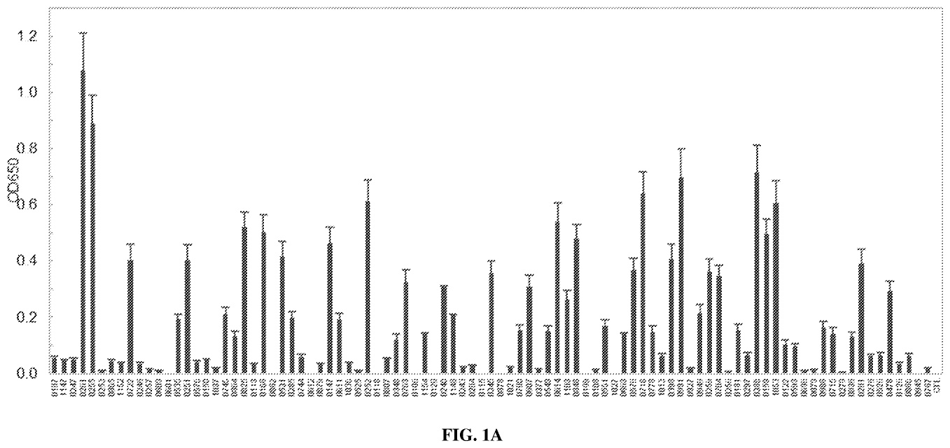

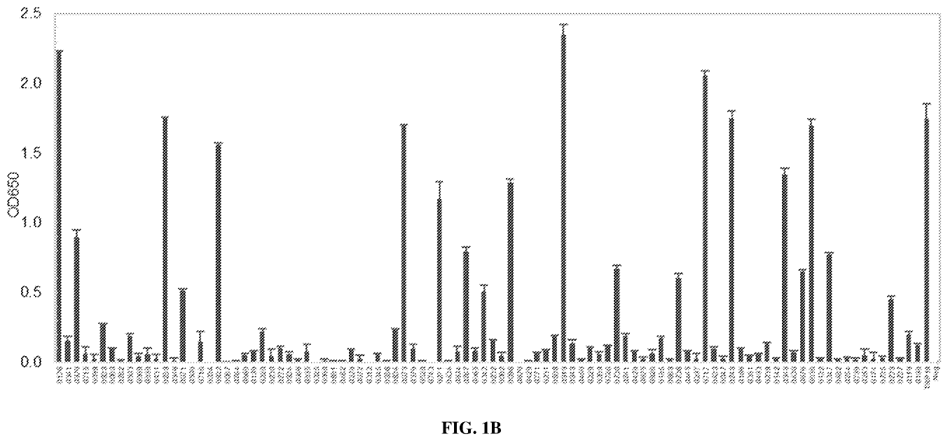

The 93 IVTT-expressed E. ch. proteins were screened for immunoreactivity by antigen capture ELISA using a single convalescent HME patient serum (IFA titer: 1600). A total of 46 (49%) E. ch. hypothetical proteins reacted with the HME patient serum (mean OD≥0.1 with background subtracted) (FIG. 1A). The 98 IVTT-expressed E. ca. proteins were similarly screened for immunoreactivity by ELISA with pooled CME dog sera (IFA titer: 1600), and 30 (31%) proteins were immunoreactive (mean OD≥0.1) (FIG. 1B). These immunoreactive E. ch. and E. ca. proteins were investigated further to determine overall immunoreactivity among a panel (n=10) of HME and CME sera, respectively.

### Identification of Major Immunoreactive E. ch. And E. ca. Hypothetical Proteins

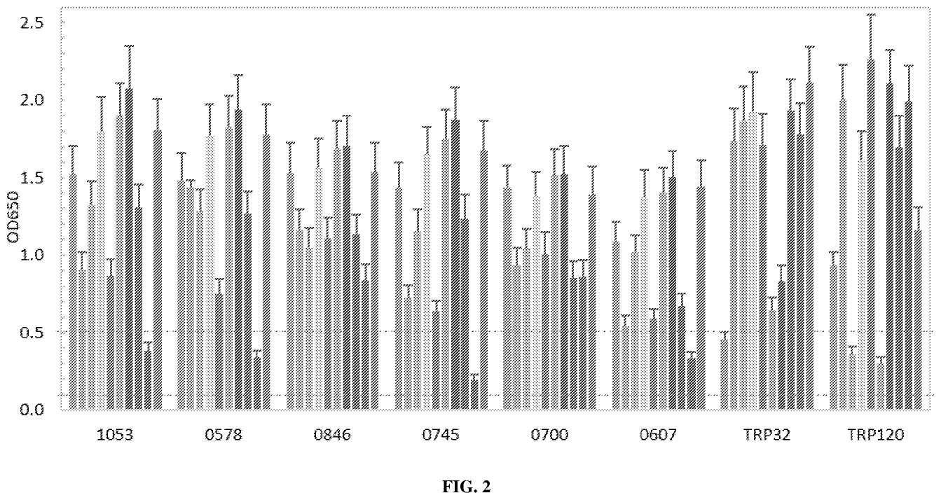

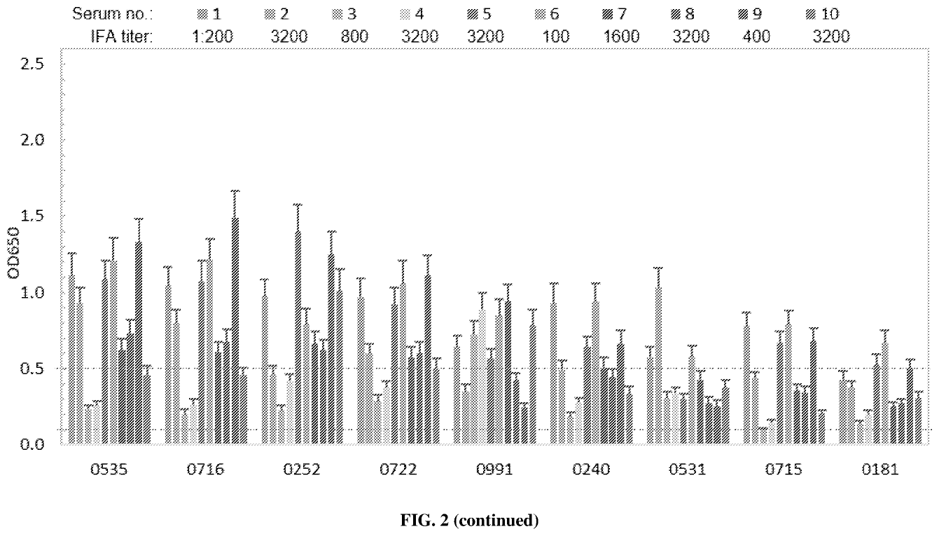

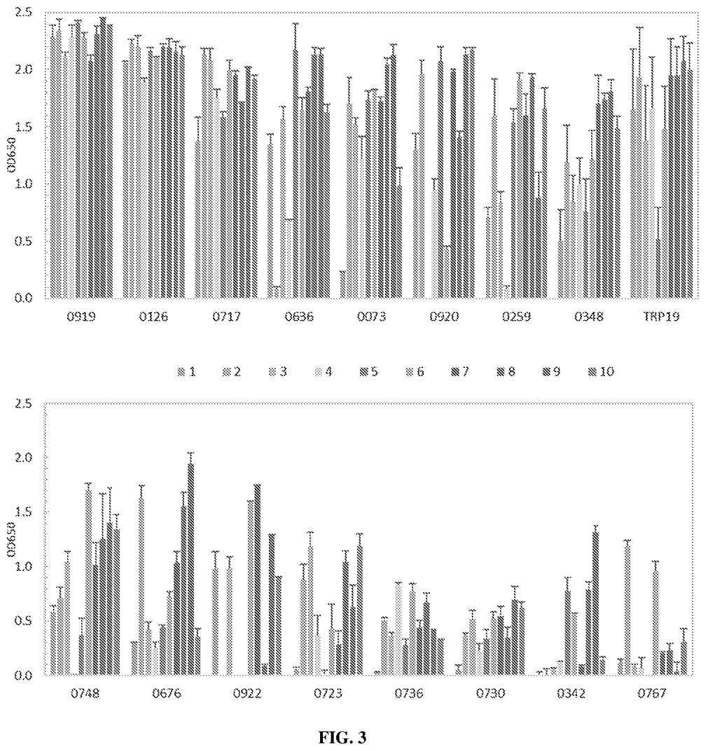

In order to define and compare the immunoreactivity of these immunoreactive E. ch. and E. ca. hypothetical proteins, an ELISA was performed with a panel of 10 HME patient or 10 CME dog sera that had detectable E. ch. or E. ca. antibodies by IFA (titers ranging from 200 to 3200). To compare the immunoreactivity of newly identified E. ch. and E. ca. immunoreactive proteins with well-defined major immunoreactive TRPs, the inventors also cloned and expressed E. ch. TRP32/TRP120 and E. ca. TRP19 by IVTT, and compared immunoreactivity of these proteins with HME and CME sera. Consistent with this previous data, E. ch. TRP32/TRP120 and E. ca. TRP19 reacted with all HME or CME sera (FIGS. 2 and 3).

Among the 46 novel E. ch. immunoreactive proteins identified, 15 (33%) proteins were recognized by all HME patient sera (FIG. 2). These proteins ranked by mean ELISA OD values are shown in Table X2. The top 6 proteins reacted strongly with most HME patient sera, which in comparison with known immunodominant proteins (TRPs) were considered immunodominant based on mean ELISA OD values (≥1.0). In addition, 6 proteins reacted strongly with the majority of HME patient sera (mean OD 0.5-0.8), and 3 proteins reacted consistently, but at lower levels with HME patient sera (mean OD 0.3-0.5). Thus, these 9 immunoreactive proteins were considered to be subdominant (FIG. 2 and Table X2). None of the HME patient sera reacted with the IVTT-expressed negative control protein (raw OD<0.08). Eight (53%) of these immunoreactive proteins were ranked in the top 100 by ANTIGENpro, indicating the substantial enrichment of antigenic proteins in the top 100 tier.

TABLE X2
Immunodominant E. ch. hypothetical protein immunoreactivity and ANTIGENpro analysis.

| | | | | E.ca. ortholog/ |
|---|---|---|---|---|
| Protein | Mean | ANTIGENpro | Antigenicity | ANTIGENpro |
| (Ech_tag no.) | ELISA OD^{a} | rank | score | rank/Immunoreactive^{b} |
| 1053 | 1.39 | 180 | 0.762 | 0846/381/− |
| 0578 | 1.39 | 137 | 0.797 | * |
| 0846 | 1.33 | 107 | 0.828 | 0242/417/− |
| 0745 | 1.23 | 23 | 0.919 | 0324/26/− |
| 0700 | 1.19 | 90 | 0.845 | * |
| 0607 | 1.00 | 92 | 0.811 | 0434/70/− |
| 0535 | 0.80 | 18 | 0.927 | 0500/24/− |
| 0716 | 0.78 | 142 | 0.790 | 0347/190/+ |
| 0252 | 0.78 | 55 | 0.874 | * |
| 0722 | 0.70 | 10 | 0.944 | 0342/80/+ |
| 0991 | 0.64 | 157 | 0.779 | 0139/32/− |
| 0240 | 0.54 | 73 | 0.856 | * |
| 0531 | 0.45 | 33 | 0.904 | * |
| 0715 | 0.45 | 194 | 0.747 | 0348/178/+ |
| 0181 | 0.37 | 170 | 0.769 | 0122/189/− |

^{a}Mean OD from 10 HME patient sera;
(*): E.ca. ortholog not identified;
^{b}(+) immunoreactive in immunoscreening;
(−) not immunoreactive in immunoscreening.

Among 30 new E. ca. immunoreactive proteins, the inventors observed that 16 (53% of 30) were recognized by most CME dog sera (FIG. 3). These proteins ranked by mean ELISA OD values are shown in Table X3. Top 8 E. ca. proteins reacted strongly with most dog sera at a level comparable to TRP19 (mean OD>1.0), thus were considered immunodominant. Another 8 E. ca. proteins reacted with most dog sera but had mean ELISA OD values<1.0 and were classified as subdominant (FIG. 3 and Table X3). None of the CME patient sera reacted with the IVTT-expressed negative control protein (raw OD<0.08). Seven (44%) of these proteins were ranked in the top 100 by ANTIGENpro.

TABLE X3
E. ca. hypothetical protein antigenicity and immunoreactivity.

| | | | | E.ch. ortholog/ |
|---|---|---|---|---|
| Protein | Mean | ANTIGENpro | Antigenicity | ANTIGENpro |
| (Ecaj_tag no.) | ELISA OD^{a} | rank | score | rank/Immunoreactive^{b} |
| 0919 | 2.29 | 106 | 0.841 | 1147/2/− |
| 0126 | 2.13 | 1 | 0.962 | 0187/1/− |
| 0717 | 1.85 | 150 | 0.804 | * |
| 0636 | 1.52 | 187 | 0.759 | 0377/95/− |
| 0073 | 1.51 | 61 | 0.887 | 0122/185/+ |
| 0920 | 1.44 | 5 | 0.947 | 1148/78/+ |
| 0259 | 1.27 | 21 | 0.928 | 0825/26/+ |
| 0348 | 1.22 | 178 | 0.767 | * |
| 0748 | 0.94 | 157 | 0.797 | * |
| 0676 | 0.87 | 186 | 0.760 | 0329/434/− |
| 0922 | 0.73 | 27 | 0.920 | * |
| 0723 | 0.60 | 222 | 0.728 | * |
| 0736 | 0.46 | 140 | 0.814 | * |
| 0730 | 0.42 | 116 | 0.831 | * |
| 0342 | 0.39 | 80 | 0.871 | * |
| 0767 | 0.32 | 75 | 0.875 | * |

^{a}Mean ELISA OD from 10 CME dog sera;
(*): E.ch. ortholog not identified;
^{b}(+): immunoreactive in immunoscreening;
(−): not immunoreactive in immunoscreening.

Several pairs of E. ch./E. ca. orthologs, including TRP32/TRP19, TRP47/TRP36, TRP75/TRP95 and TRP120/TRP140, are major immunoreactive proteins. Although 10 E. ca. and 7 E. ch. orthologs were identified in IMG that corresponded with the new major immunoreactive proteins, none of ortholog pairs exhibited similar immunoreactivity (Table X2 and Table X3). 3 E. ca. and 3 E. ch. orthologs reacted with a few of CME dog and HME patient sera, respectively, but none of these orthologs reacted consistently with HME/CME sera. These findings suggest that the antibody epitopes in the new proteins are not conserved among corresponding ortholog pairs from E. ch. and E. ca., unlike the linear antibody epitopes defined in orthologs previously reported (McBride et al., 2010).

Bioinformatic Analysis of E. ch. And E. ca. Immunoreactive Proteins.

The inventors observed that among the 15 E. ch. and 16 E. ca. new immunoreactive proteins, 10 (67%, E. ch.) and 13 (81%, E. ca.) were small (≤250 amino acids) (Table X4 and Table X5). Notably, tandem repeats were found in 3 E. ch. proteins (Ech_0700, 0252 and 0531) and 1 E. ca. protein (Ecaj_0126), similar to other well-known ehrlichial immunoreactive TRPs. A comprehensive bioinformatic analysis of these proteins was performed using multiple online prediction tools. By TMHMM 2.0 server, 9 (60%) E. ch. and 7 (44%) E. ca. proteins were predicted to contain at least 1 transmembrane helix. However, using SignalP 5.0, a standard secretory signal peptide, which is transported by the Sec translocon and cleaved by signal peptidase I, was identified in only 1 protein (0846). Moreover, SecretomeP 2.0 predicted 6 E. ch. and 5 E. ca. proteins to be secreted by a non-classical (i.e., not signal peptide directed) mechanism. Since type I and type IV secretion systems (T1SS and T4SS) are present in E. ch. and E. ca., the inventors examined these proteins as possible T1 and T4 substrates. Sequence analysis did not identify a consensus type IV secretory motif R—X(7)-R—X—R—X—R (SEQ ID NO: 76) in any of the proteins (Vergunst et al., 2005). Two E. ca proteins (0126 and 0259) were predicted to be type IV substrates by S4TE 2.0, a new algorithm that predicts type IV effector proteins (Noroy et al., 2007); however, none of the E. ch. proteins were predicted to be type IV substrates. In contrast, statistical analysis of the last 50 C-terminal residues of these proteins identified a putative type I secretion signal (LDAVTSIF-enriched (SEQ ID NO: 77); KHPMWC-poor (SEQ ID NO: 78)) described previously (Delepelaire, 2004), supporting the idea that the majority of these proteins might be type I secreted substrates. The predicted type IV substrate Ecaj_0259 showed the smallest difference between the residue occurrences of LDAVTSIF (SEQ ID NO: 77) (36%) and KHPMWC (SEQ ID NO: 78) (24%) in the last 50 C-terminal amino acids. In addition, these proteins were further examined using the recently reported PREFFECTOR server, which identifies all effectors regardless of the secretion system using a feature-based statistical framework (Dhroso et al., 2018). PREFFECTOR prediction identified 10 (66%) E. ch. and 10 (63%) E. ca. proteins as effectors (probability threshold=0.8). This analysis supports the idea that many of these proteins are small type I secreted effectors, of which 52% contain a transmembrane domain (Table X4 and Table X5). Further experiments could be performed to experimentally validate if they are T1SS substrates.

TABLE X4
Predicted features of novel E. ch. immunodominant hypothetical proteins.

| | Amino | | | | | |
|---|---|---|---|---|---|---|
| | acids/ | | Transmembrane | | | |
| Protein | Mass | Tandem | domains | Secretion | T4S | Effector |
| (Ech_tag no.) | (kD) | repeats | (TMHMM) | (SecretomeP) | (S4TE) | (Preffector) |
| 1053 | 193/22 | − | + | − | − | + |
| 0578 | 185/21 | − | − | − | − | − |
| 0846 | 171/19 | − | − | +^{a} | − | + |
| 0745 | 118/13 | − | − | + | − | + |
| 0700 | 192/20 | + | − | + | − | + |
| 0607 | 322/38 | − | − | − | − | + |
| 0535 | 186/21 | − | − | + | − | + |
| 0716 | 367/41 | − | + | − | − | − |
| 0252 | 364/40 | + | + | − | − | − |
| 0722 | 190/21 | − | + | + | − | + |
| 0991 | 710/81 | − | + | − | − | + |
| 0240 | 158/18 | − | + | − | − | − |
| 0531 | 175/20 | + | + | + | − | + |
| 0715 | 551/61 | − | + | − | − | − |
| 0181 | 103/12 | − | + | + | − | + |

^{a}Signal peptide predicted by SignalP

TABLE X5
Predicted features of novel E. ca. immunodominant hypothetical proteins.

| | Amino | | | | | |
|---|---|---|---|---|---|---|
| | acids/ | | Transmembrane | | | |
| Protein | Mass | Tandem | domains | Secretion | T4S | Effector |
| (Ecaj_tag no.) | (kD) | repeats | (TMHMM) | (SecretomeP) | (S4TE) | (Preffector) |
| 0919 | 120/14 | − | − | + | − | + |
| 0126 | 671/78 | + | − | + | + | + |
| 0717 | 226/25 | − | + | − | − | + |
| 0636 | 98/11 | − | − | − | − | − |
| 0073 | 92/10 | − | − | + | − | − |
| 0920 | 182/20 | − | − | + | − | + |
| 0259 | 368/41 | − | − | + | + | + |
| 0348 | 535/59 | − | + | − | − | − |
| 0748 | 121/13 | − | + | − | − | − |
| 0676 | 229/26 | − | − | − | − | + |
| 0922 | 133/15 | − | − | − | − | + |
| 0723 | 248/28 | − | + | − | − | − |
| 0736 | 188/21 | − | + | − | − | + |
| 0730 | 165/18 | − | − | − | − | − |
| 0342 | 217/24 | − | + | − | − | + |
| 0767 | 91/10 | − | + | − | − | + |

E. ch. And E. ca. Immunoreactive Protein Antibody Epitopes

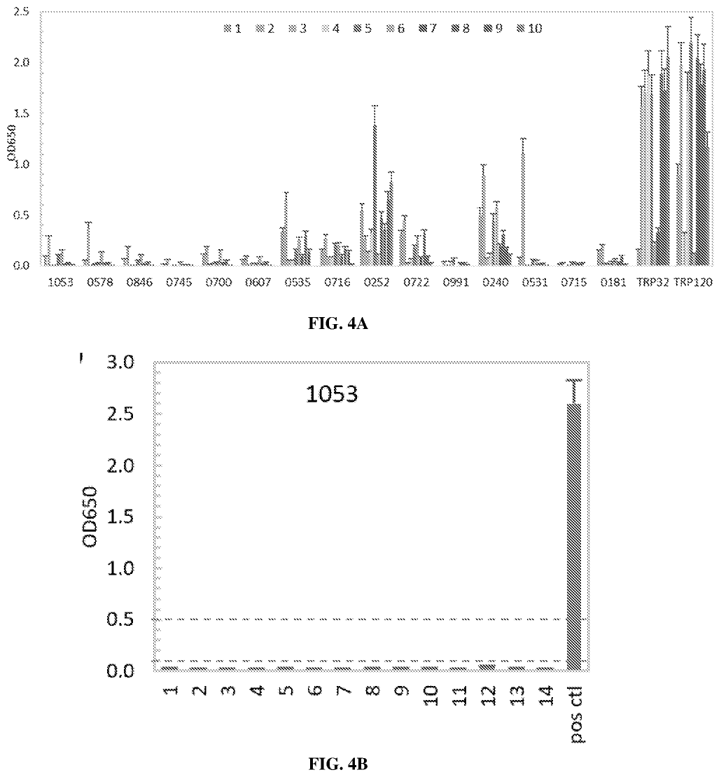

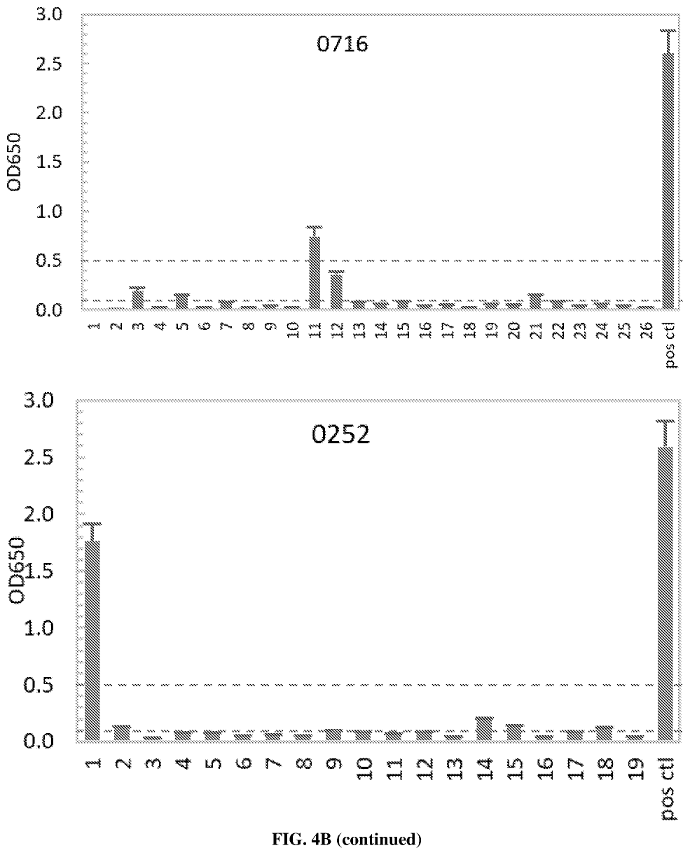

The number of major immunoreactive E. ch. proteins identified by immunoblotting is small and well-defined (McBride and Walker, 2010). Thus, to understand how these new immunoreactive proteins are not apparent by immunoblot, the inventors investigated the possibility of conformation-dependent antibody epitopes. To examine this question, the immunoreactivity of native proteins (IVTT products) was compared with that of denatured proteins (IVTT products treated by urea) by ELISA with the same panel of sera from 10 HME patients (FIG. 4A). After denaturation, 4 E. ch. immunoreactive proteins (0745, 0607, 0991 and 0715) did not react with any patient serum; 5 proteins (1053, 0578, 0846, 0700 and 0181) reacted weakly with 1-3 patient sera; 5 proteins (0535, 0716, 0252, 0722 and 0240) reacted with most patient sera but at a substantially lower level compared to native IVTT proteins. One protein (0531) reacted strongly with a single patient serum but did not react with the other 9 sera. However, the immunoreactivity of major immunoreactive TRP32 and TRP120 was not affected by denaturation, consistent with previous reports demonstrating that TRPs contain major linear epitopes (FIG. 4A) (McBride and Walker, 2010). These results indicate that these new E. ch. immunoreactive proteins are defined by conformation-dependent antibody epitopes.

Synthetic peptides have been used to map linear epitopes in E. ch. TRPs (Luo et al., 2009; Luo et al., 2008; McBride et al., 2011). The inventors used this approach to further determine if new E. ch. immunoreactive proteins contain significant linear epitopes. Overlapping polypeptides (20-25 amino acids; 6 amino acid overlap) were synthesized to cover the sequence of 13 E. ch. immunoreactive proteins (Table X2; except Ech_0991 and 0715). The HME patient serum used in initial screening was used to probe all peptides by ELISA (FIG. 4B). Several peptides from Ech_0716 (peptides 3, 5, 11, 12, and 21) and 0252 (peptides 1, 14 and 15) reacted with the HME serum. Overlapping peptides representing the remaining 11 proteins, such as Ech_1053, did not react with the HME patient serum, supporting the conclusion that a majority of these new E. ch. immunoreactive proteins do not contain major linear epitopes, a finding consistent with ELISA using native and denatured IVTT products (FIG. 2 and FIG. 4A).

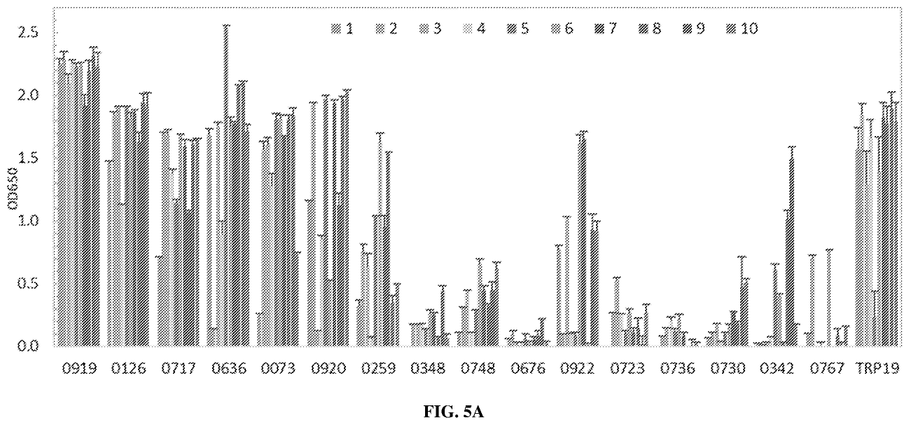

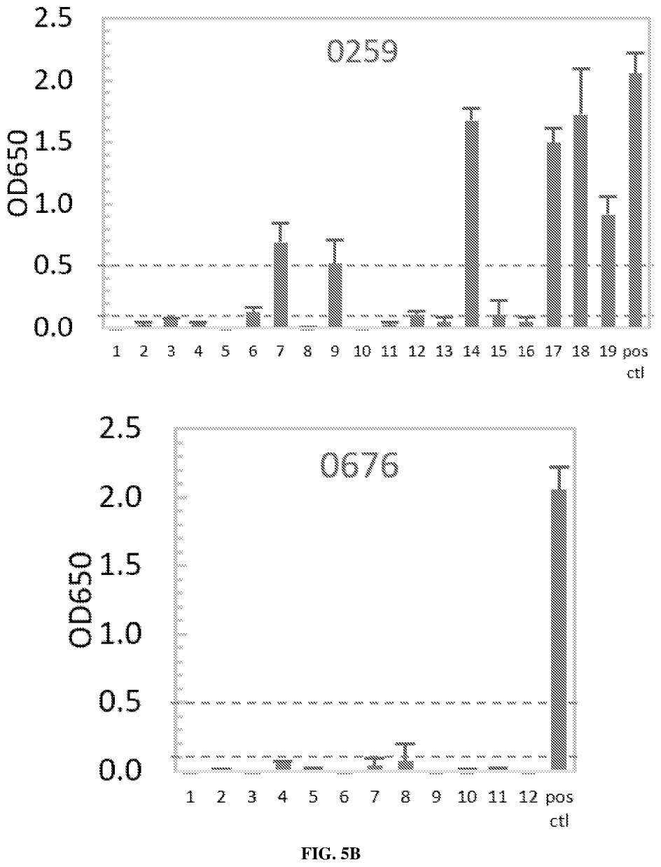

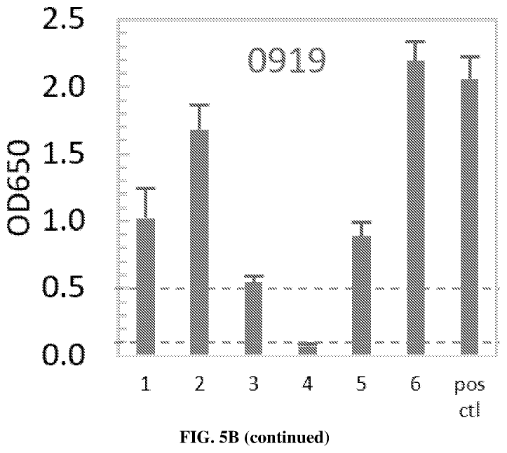

In order to examine epitope conformation-dependence of new E. ca. immunoreactive proteins, the immunoreactivity of native proteins and denatured IVTT products was compared by ELISA with 10 CME dog sera (FIG. 5A). After denaturation, 7 E. ca. proteins, including Ecaj_0348, 0748, 0676, 0723, 0736, 0730 and 0767 did not react with most dog sera or reacted with sera at a substantially lower level compared to native IVTT proteins; however, the immunoreactivity of other 9 new E. ca. proteins was not reduced substantially, similar to well-defined E. ca. major immunoreactive protein TRP19 (FIG. 5A). Thus, these results indicate that 7/16 new E. ca. immunoreactive proteins have conformation-dependent antibody epitopes.

Additionally, conformational dependence was investigated using overlapping synthetic peptides to identify linear epitopes in 3 E. ca. proteins (FIG. 5B). By ELISA, some peptides of Ecaj_0259 (peptides 7, 9, 14, 17, 18 and 19) and 0919 (peptide 1, 2, 3, 5 and 6) reacted strongly with the CME dog serum, suggesting the presence of major linear epitopes in these 2 proteins. None of Ecaj_0676 peptides reacted with the dog serum, suggesting the absence of linear epitopes. These results support the conclusion that some new E. ca. immunoreactive proteins contain major linear epitopes while some others contain conformational epitopes (FIG. 3 and FIG. 5A).

Conformational dependence was also investigated some of these E. ch. and E. ca. immunoreactive proteins by dot immunoblot (FIG. S2). The immunoreactivity of native proteins was compared with that of denatured proteins using an HME or CME serum. After denaturation, Ech_0745 did not react with the HME serum and Ech_0535 and 0716 reacted weakly with the serum, whereas Ech_0252 still reacted strongly with the serum but at a lower level compared to native proteins. These results are consistent with ELISA data in FIG. 4A and support the conclusion that these new E. ch. immunoreactive proteins are defined by conformation-dependent antibody epitopes. After denaturation, Ecaj_0919 still reacted strongly with the CME serum but Ecaj_0636, 0073 and 0676 did not react with the serum. The results of Ecaj_0636 and 0073 are not consistent with ELISA data in FIG. 5A, suggesting that these 2 proteins contain conformational epitopes that could refold on ELISA plate and recover the immunoreactivity after denaturing. This data supports the conclusion that the majority of these new E. ca. immunoreactive proteins contain conformational epitopes.

The first immunoreactive E. ch. proteins (GroES/EL) were molecularly characterized in 1993. Since that time, molecular and proteomic approaches used to identify major immunoreactive proteins of Ehrlichia spp. have revealed a small subset of proteins (TRPs, Anks and OMPs) defined by immunodominant linear antibody epitopes (McBride and Walker, 2011). These proteins are easily identifiable on E. ch. or E. ca.-infected mammalian cell immunoblots and have been the primary focus of immunomolecular characterization studies of these pathogens (McBride and Walker, 2011; Chen et al., 1997; McBride et al., 2003). In these studies, bioinformatic prediction was used to rank the top 250 E. ch. and E. ca. antigenic proteins and further investigated the proteins (−40%) contained in this group that have unknown function (hypothetical). Cell-free high-throughput IVTT ORF expression was used and was observed to be sufficient to overcome a major barrier in identifying the immunoreactive ehrlichial proteins. Combining these approaches, the inventors identified many previously undiscovered immunodominant and subdominant ehrlichial proteins, most characterized by small size (<250 aa) and conformation-dependent immunoreactivity. These proteins, which have remained undefined and can be included in vaccine compositions and used in diagnostic methods.

In these experiments, new-generation computational and biotechnical approaches were used, including bioinformatic prediction (reverse vaccinology) to prioritize candidate screening, gene synthesis to overcome issues associated with low-throughput manual gene cloning, IVTT to express proteins in native conformation, particularly those that are not amenable to cell-based expression systems, and high-throughput ELISA immunoscreening to rapidly and accurately identify novel immunoreactive proteins. The inventors observed that E. ch. and E. ca. proteins could be expressed by IVTT, and many of these proteins likely could not be expressed in a cell-based expression system. With IVTT, the ORF expression levels varied; however, the expression levels did not appear to impact the identification of immunoreactive proteins since many proteins with lower IVTT expression levels showed strong immunoreactivities with patient sera (e.g., Ech_0745, 0607, 0578 and Ecaj_0730, 0717, 0748).

A bioinformatic approach was utilized to help identify and prioritize E. ch. and E. ca. immunoreactive proteins. ANTIGENpro has been utilized to identify antigens that might generate a protective humoral immune response; however, in silico approaches are limited and in vitro testing is normally required to determine in antigenicity is actually observed. A cutoff was used to develop a list of proteins with the highest antigenic scores in order to improve positive hits and increase the rate at which the inventors might identify the most promising prospects. In this analysis, ANTIGENpro ranked known antigenic/protective proteins such as TRPs and OMPs in the top 250 list (Table X1).

The E. ch. and E. ca. genomes contain 426 (38%) and 238 (25%) genes, respectively, that encode proteins of unknown function so far. The proportion of E. ch. hypothetical proteins represented in the genome is nearly double that of E. ca. Notably, nearly half of the E. ca. hypothetical ORFs (n=98) were predicted by ANTIGENpro as highly antigenic; however, only 22% (n=93) of the E. ch. hypothetical ORFs were predicted as highly antigenic. The majority of known ehrlichial immunoreactive proteins, including TRPs, were initially classified as hypothetical proteins. However, recent studies have revealed the functional aspects of TRPs, and it is now established that they are secreted effectors that interact with an array of host proteins and have various distinct functions during infection (Chin et al., 2014; Luo and McBride, 2003; Luo et al., 2018; Wakeel et al., 2009). Some hypothetical proteins have also been observed immunoreactive proteins in other intracellular pathogens (Cruz-Fisher et al., 2011; Liu et al., 2019).

The E. ch. and E. ca. genomes have a large proportion of predicted ORFs (˜45% and 35%, respectively) that encode small proteins (<250 aa). In fact, ˜25% of the E. ch. ORFs encode proteins <100 aa. Very few of these proteins have been investigated, and the smallest known immunoreactive protein is E. ca TRP19 which is 160 aa. The reasons for the large proportion of small proteins encoded by these genomes and their role in pathobiology are unknown. In this study, the inventors identified many (23/31; 74%) small (<250 aa) novel hypothetical immunoreactive proteins of E. ch. and E. ca. Nineteen (61%) of the immunoreactive proteins of E. ch. and E. ca. were <200 aa and three proteins were <100 aa. Conventional gel electrophoresis would not resolve such proteins well, or in many instances these proteins would be eliminated from the gel depending on the gel composition and electrophoresis conditions. These findings suggest that there has been a large group of important immunoreactive proteins that may have remained undefined in part due to difficulties in resolving such proteins with standard gel electrophoretic approaches.

All previously characterized E. ch. and E. ca. immunoreactive proteins contain major linear epitopes (McBride and Walker, 2010). Yet, conformation-dependent epitopes predominated in this study, suggesting that previous approaches used to identify immunoreactive proteins were effective in revealing the immunoreactive proteins with linear antibody epitopes while leaving many immunoreactive proteins undiscovered. Identification of proteins with conformational epitopes was achieved in the experiments in this example using IVTT, and it was observed that this method was capable of expressing proteins in native conformation in solution. Using IVTT, the inventors exposed conformation-dependent epitopes previously concealed, revealing a large group of antigenic proteins. To the inventors' knowledge, such a large abundance of proteins with conformational epitopes has never been reported previously in other pathogens and indicates that the Ehrlichia immunomes have a predominance of epitopes with conformation-dependence.

Conformational antibody epitopes have been experimentally identified in human pathogens, mostly reported in viruses, such as functional epitopes on hepatitis E virions/capsids, the orf virus major envelope protein B2L and dengue virus envelope E glycoprotein (Yu et al., 2019; Andrade et al., 2019; He et al., 2020). A few conformation-dependent epitopes have also been described in bacterial proteins, such as E. ch. TRP32, Yersinia pseudotuberculosis OmpF porin, and Campylobacter jejuni membrane protein Cj1621 (Luo et al., 2008; Luo et al., 2019; Portnyagina et al., 2018). These proteins have been demonstrated to play an important role in pathogen infection and eliciting host antibody response. Both linear and conformational antibody epitopes are essential in stimulating immunity; however, it has been estimated that more than 90% of B-cell epitopes are conformational, since less than 10% of antibodies raised against intact proteins react with peptide fragments derived from the parent protein (Portnyagina et al., 2018). The frequency of conformation-dependent epitopes in Ehrlichia spp. revealed in this investigation demonstrates the importance of such epitopes in generating an immune response and further highlights the need to fully identify and define the immunodeterminants for effective vaccines to be developed.

Ehrlichia spp. infect arthropod and mammalian hosts, and this host infection dynamic may have also contributed to the difficulties in uncovering these immunoreactive proteins. Prior to this study, antigens that had been discovered are known to be highly expressed in mammalian cells (Kuriakose et al., 2011). Previously, most investigations have relied on Ehrlichia-infected human/canine cells instead of tick cell cultures for antigen discovery; however, it has been demonstrated that differential expression of Ehrlichia antigenic proteins occurs in arthropod vs. mammalian hosts (Kuriakose et al., 2011; Seo et al., 2008; Singu et al., 2006). Upregulated gene expression of a large number of Ehrlichia hypothetical proteins was reported in tick cells compared to human cells (Kuriakose et al., 2011). Others have demonstrated divergent protein immunoreactivity in tick vs human cell cultivated E. ch., illuminating the differences in E. ch. proteomes from distinct host cell environments (Seo et al., 2008). Without wishing to be bound by any theory, the inventors anticipate that many of these newly identified Ehrlichia antigens are expressed in tick cells, not in mammalian cells, and thus have escaped identification and characterization. A previous study using one-dimensional electrophoresis identified several immunoreactive hypothetical proteins expressed in mammalian and/or tick cells (Seo et al., 2008); however, although these proteins were classified as antigenic in these experiments, they were not identified as immunoreactive in this study. Differences observed between the previous study and the current investigation could be related to the approach of excising proteins from a gel for mass spectrometry that may include comigrating proteins rather than the direct gene expression approach used herein. In addition, previous studies have used sera from needle-inoculated mice to identify immunoreactive E. ch. proteins. This study used convalescent sera from tick-transmitted HME patients and CME dogs to identify immunoreactive proteins. Moreover, the methods used herein were observed to be capable of identifying proteins that contain conformational epitopes. The results observed herein provide evidence that the immunoreactive proteins provided herein can be used in immunodiagnostics (e.g., for detection of early antibodies that are elicited by tick-expressed ehrlichial proteins) or in a transmission-blocking or infection-blocking subunit vaccine.

Most of the immunoreactive proteins previously identified from E. ch. and E. ca. consist of ortholog pairs, such as TRP32/TRP19, TRP47/TRP36, TRP75/TRP95, TRP120/TRP140 and Ank200/Ank200, all of which contain major linear epitopes, suggesting that ehrlichiae have similar orthologous immunomes (Lina et al., 2016). However, in contrast to previously established similarity between immunoreactive orthologs, ortholog pairs in this study did not react similarly and consistently with antibodies in sera from infected patients/dogs. Moreover, many of the new E. ch./E. ca. immunoreactive proteins do not have corresponding orthologs. Since most of the new proteins exhibited conformational epitopes, this difference highlights a divergence in antibody recognition that is fundamentally different from previously defined linear epitopes in major immunoreactive proteins of Ehrlichia. These results further demonstrated that E. ch. and E. ca. have vastly different conformational immunomes that are not shared between the species, a finding in contrast with previously defined linear epitope containing proteins. This newly recognized diversity in immunomes has potential importance in development of effective vaccines and provides new insight into the feasibility of developing cross protective vaccines.

Tandem repeats were identified in four new immunoreactive proteins (3 in E. ch. and 1 in E. ca.) and this observation further highlights the importance of ehrlichial tandem repeat proteins as targets of the host immune response. In addition, although many of the new immunoreactive proteins of E. ch. are predicted to be secreted, they also contain transmembrane domains which are considered a feature of membrane proteins. The significance of transmembrane domains in secreted effector proteins remains to be determined, but this is an interesting and unique feature that, to the inventors' knowledge, has not been previously described.

In this study, the majority of new immunoreactive proteins were predicted to be secreted, but only 2 E. ch. proteins (Ecaj_0126 and 0259) were predicted to be T4S substrates by S4TE. Ehrlichia spp. have type I and type IV secretion systems, which are both common among gram-negative bacteria. Substantial emphasis has been placed on identification of T4 effectors in many different bacteria including Ehrlichia. TRPs and Ank200 have also been identified as T1SS substrates that are major immunoreactive proteins (Wakeel et al., 2011). TRPs interact with multiple host proteins associated with conserved cell biological processes, including cell signaling, transcriptional regulation, vesicle trafficking, cytoskeleton organization and apoptosis (Lina et al., 2016; Dunphy et al., 2013). Without wishing to be bound by any theory, the proteins provided herein might be involved in a variety of different interactions with the host cell during infection. The fact that these Ehrlichia T1SS substrates are immunodominant proteins suggests that such effectors are predominantly targeted by the host immune response, which may be related to the importance of neutralizing their functional properties to limit infection. Further studies could be performed to confirm whether these new immunoreactive E. ch. proteins are indeed type I-secreted effectors and to determine their role in ehrlichial pathobiology.

The new Ehrlichia proteins identified in this study significantly expand the number of identified major immunoreactive proteins and highlights the potential importance of conformational antibody epitopes in immunity and differential expression of ehrlichial proteins in mammalian and tick hosts. Major immunoreactive proteins are provided herein that have immunoreactivity that rival the highly immunoreactive and immunogenic TRPs. These new immunoreactive proteins can be used in the diagnosis of HME and CME, or as markers to distinguish vaccinated from non-vaccinated subjects. One or more of the immunoreactive proteins provided herein can be included in a subunit vaccine, e.g., against HME and/or CME.

### Example 2

### Materials and Methods

The following methods were used for the experiments of Example 1.

ANTIGENpro prediction. The E. ch. (Arkansas strain) and E. ca. (Jake strain) ORFeomes were analyzed by ANTIGENpro, a sequence-based and alignment-free predictor of protein antigenicity (Magnan et al., 2010). The predictions are made by a two-stage architecture based on multiple representations of the primary sequence and five machine learning algorithms. A final score (0 to 1) defines the antigenic probability, with a higher score correlating with increased antigenicity. A prediction threshold of ˜0.7, which provides maximum sensitivity and specificity, was used to identify the most highly antigenic proteins in E. ch. and E. ca.

Gene synthesis. E. ch. or E. ca. genes in this study are available by locus tag identification in the Integrated Microbial Genomes (IMG). The open reading frames (ORFs) were obtained by either PCR amplification or chemical gene synthesis. For PCR, E. ch. (Arkansas) or E. ca. (Jake) was propagated and purified for genomic DNA preparation as previously described (Kuriakose et al., 2011; McBride et al., 2001; McBride et al., 1996). Oligonucleotide primers for the amplification of the gene fragments were designed manually or by PrimerSelect (Lasergene 13, DNASTAR, Madison, Wis.) according to the sequences and synthesized (Integrated DNA Technologies, Coralville, Iowa). PCR was performed with PCR HotMaster Mix (Eppendorf, Westbury, N.Y.) using E. ch. or E. ca. genomic DNA as the template. The thermal cycling profile was: 950 C for 3 min, 30 cycles of 94° C. for 30 s, annealing temperature (1° C. less than the lowest primer Tₘ) for 30 s, and 72° C. for the appropriate extension time (1 min/1000 base pairs) followed by a 72° C. extension for 10 min and a 4° C. hold. Synthesis of E. ch and E. ca. genes was performed by GenScript (Piscataway, N.J.) or Biomatik (Wilmington, Del.)

HME and CME antisera. HME patient sera were kind gifts from the Centers for Disease Control and Prevention (Atlanta, Ga.), Vanderbilt University (Nashville, Tenn.), Washington University and St. Louis Children's Hospital (St. Louis, Mo.). CME dog sera were obtained from naturally infected dogs from the United States and Colombia.

IVTT. In vitro expression of ehrlichial proteins was performed using the S30 T7 high-yield protein expression system (Promega, Madison, Wis.), an E. coli extract-based cell-free protein synthesis system which can produce a high level of recombinant protein in vitro. Briefly, the ehrlichial ORFs were cloned in pIVEX-2.3d or pET-14b vector containing T7 promoter/terminator and a 6×His-tag sequence. The recombinant plasmid was mixed with a E. coli extract and a reaction premix that contain all necessary components for transcription and translation, such as T7 RNA polymerase and ribosomal machinery, followed by the incubation at 37° C. for 2 h. The IVTT expression of ehrlichial ORFs was confirmed by dot immunoblotting IVTT-expressed products (1 μl each) on nitrocellulose. The membrane was incubated with horseradish peroxidase (HRP)-labeled His-tag mouse antibody (1:500; GenScript) in Tris-buffered saline (TBS) with 3% nonfat dry milk and 0.1% Tween 20 for 1 h at room temperature. The membrane was washed 3 times with TBS, and the protein was visualized after adding TMB 1-component substrate (Kirkegaard & Perry Laboratories, Gaithersburg, Md.) and incubating for 15 min. IVTT-expressed ehrlichial proteins was purified by MagneHis protein purification system (Promega) according to the instructions from the manufacturer, and the immunoreactivity was examined by dot immunoblotting using an HME or CME antiserum.

ELISA immunoscreening. A His-tag antigen capture ELISA was used to screen E. ch. and E. ca His-tag IVTT-expressed proteins for immunoreactivity. Briefly, His-tag antibody plates (GenScript) were blocked with 100 μl of StartingBlock™ (PBS) blocking buffer (Thermo Fisher) with 2% nonfat milk for 20 min and washed twice with 200 μl of phosphate-buffered saline containing 0.05% (v/v) Tween 20 (PBST). The plates were coated with 50 μl of diluted (1:50) IVTT reaction mixture in dilution buffer (blocking buffer with 2% nonfat milk and 0.05% Tween 20) and incubated overnight at 4° C. The plates were washed five times with PBST using an Immunowash 1575 microplate washer (Bio-Rad, Hercules, Calif.). HME patient or CME dog sera diluted (1:200) in dilution buffer were added to each well (50 μl) and incubated for 1 h. ELISA plates were washed again, and 50 μl of alkaline phosphatase-labeled rabbit anti-human IgG (H+L) secondary antibody (1:5,000; Abcam, Cambridge, Mass.) in dilution buffer was added, and incubated for 1 h. After final washes, 100 μl of BluePhos substrate (Kirkegaard & Perry) was added and the plates were incubated in the dark for 30 min. Optical density (OD) was determined using a VersaMax microplate reader (Molecular Devices, Sunnyvale, Calif.) at A₆₅₀ and data analyzed by Softmax Pro 7 (Molecular Devices). Immunoreactivity of denatured IVTT-expressed proteins was examined similarly except the dilution buffer contained 4 M urea, and the mixture was incubated for 10 min at 99° C. before coating the ELISA plates.

Peptide ELISA. Peptide ELISAs to identify linear antibody epitopes in E. ch. and E. ca. immunoreactive proteins were performed as previously described using overlapping peptides (6 amino acids overlapped) commercially synthesized by Bio-Synthesis (Lewisville, Tex.) or Biomatik (Luo et al., 2009). All peptides were supplied as a lyophilized powder and resuspended in molecular biology grade water (1 mg/ml). ELISA OD values represent the mean OD reading from 3 wells (±SD) after background subtraction. Since negative controls generally had raw readings of <0.08 OD, a sample OD of ≥0.1 was considered positive and ≥0.5 a strong positive after subtracting negative control reading.

Indirect fluorescent-antibody assay (IFA). The antibody titers of HME patient and CME dog sera were determined as described previously (Luo et al., 2011). Antigen slides were prepared from THP-1 cells infected with E. ch. (Arkansas) or DH82 cells infected with E. ca. (Jake). Sera were diluted two-fold in PBS, starting at 1:100.

### Example 3

### Immunoreactive Protein Repertoires of Ehrlichia chaffeensis and E. canis Reveal the Dominance of Hypothetical Proteins and Conformation-Dependent Antibody Epitopes

Expression and Immunoscreening of E. ch. And E. ca. Proteins

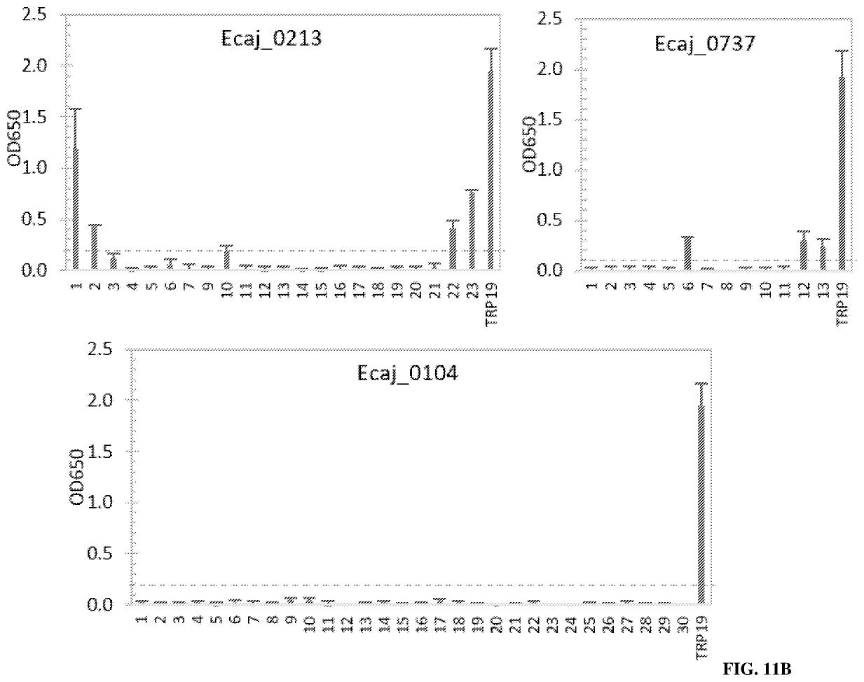

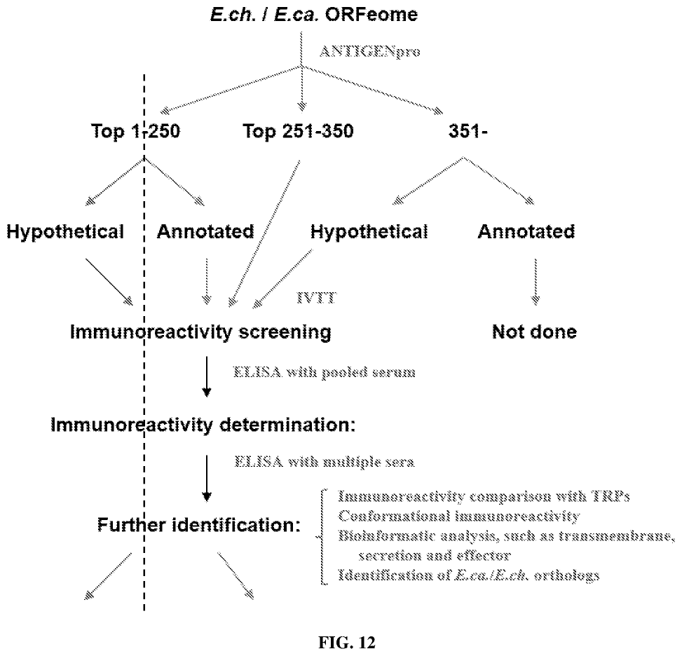

Previously we predicted the antigenicity of 1105 E. ch. (Arkansas strain) and 925 E. ca. (Jake strain) open reading frames (ORFs; excluding RNA genes and pseudogenes) by ANTIGENpro and obtained respective antigenicity scores (between 0 and 1). The enrichment efficiency of ANTIGENpro was validated by known protective antigens of E. ch. and E. ca (i.e., TRPs and OMPs). We also investigated the immunoreactivity of ˜100 hypothetical proteins distributed in the top 250 in each respective ORFeomes (21). In order to further extend the knowledge regarding immunoreactive protein repertoires of E. ch. and E. ca., The inventors investigated proteins in top 350 (including both hypothetical and annotated proteins, but excluding known antigens and ribosomal proteins) and remaining hypothetical proteins present in E. ch. and E. ca. ORFeomes (FIG. 12). The antigenicity score threshold (˜0.6) leading to the identification of the top 350 E. ch. and E. ca. proteins provided a balance between high sensitivity and specificity in predicting protein antigenicity (29). In addition, the remaining E. ch. and E. ca. hypothetical proteins in the ORFeome were also included regardless of ANTIGENpro rank. Due to different pipelines used for genomic annotation, there were some differences in gene assignments in the E. ch. and E. ca. ORFeomes between Integrated Microbial Genomes (IMG) and GenBank. Several hypothetical proteins (including proteins with domain of unknown function [DUF]) predicted by GenBank, but not by IMG, were also included. A few very small E. ch. proteins (<42 aa) predicted by IMG were removed by GenBank pipeline and were not included in this study. Therefore, the total number of proteins in E. ch. ORFeome was adjusted to 882.

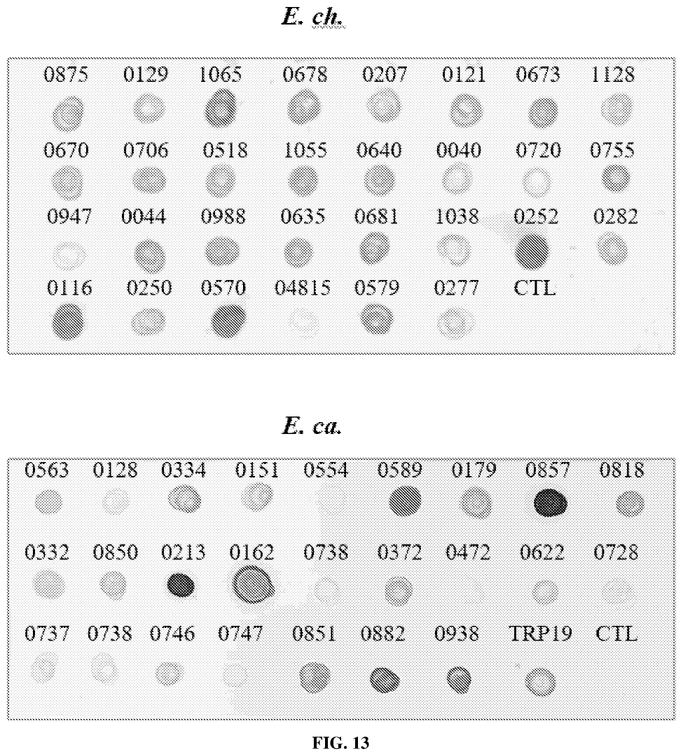

Whole genome annotations for E. ch. and E. ca. currently assign 176 (20%) and 230 (25%) ORFs as hypothetical or DUF-containing. Within the ANTIGENpro top 350, we have previously investigated 93 E. ch. and 98 E. ca. proteins (21). Therefore, in this study we further examined the immunoreactivity of 320 E. ch. and 314 E. ca. proteins in total, including all remaining hypothetical proteins (n=104 and n=124, respectively) regardless of ANTIGENpro ranking which were not examined in our previous investigation. A cell free in vitro transcription and translation (IVTT) system was used to express the ORFs and protein expression was confirmed by dot blot of selected proteins (30 from E. ch. and 25 from E. ca.) (FIG. 13). Although the protein expression levels varied, the identification of immunoreactive proteins was not influenced by expression levels due to the saturation of ELISA plate wells by IVTT-expressed proteins as confirmed by our previous investigation (21).

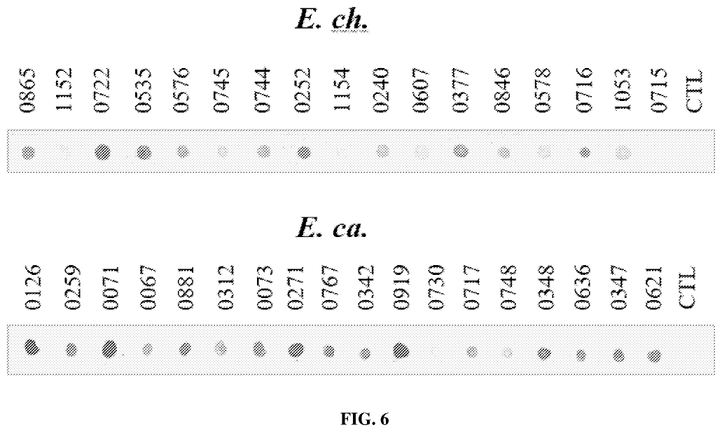

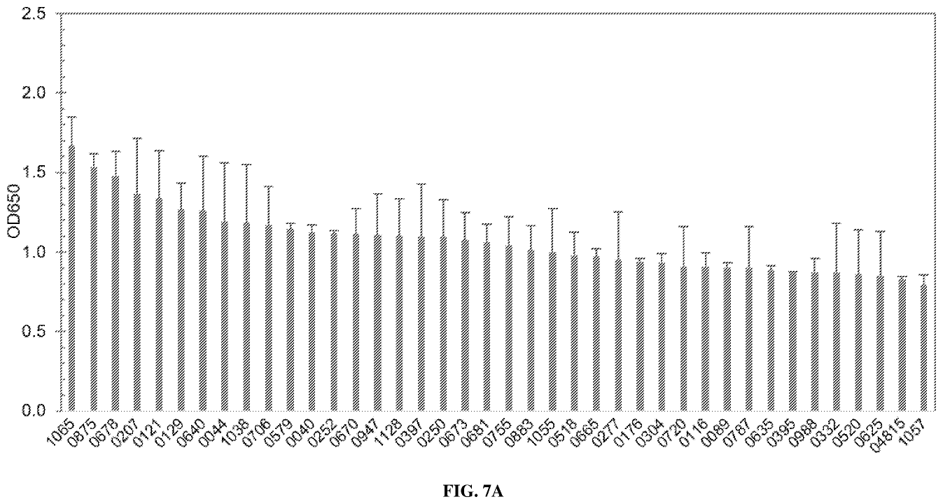

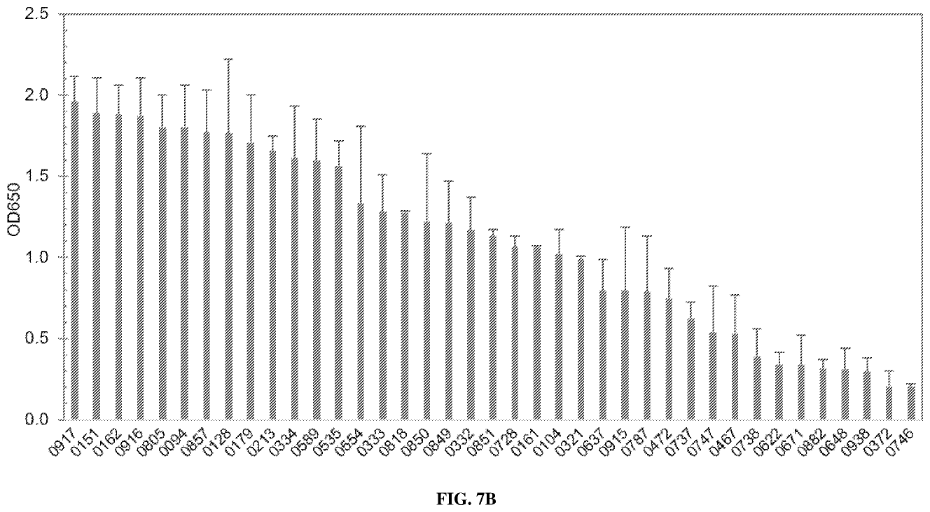

The 320 IVTT-expressed E. ch. proteins were screened for immunoreactivity by antigen capture ELISA using pooled convalescent HME sera (IFA titer: 1600), and a total of 118 (37%) E. ch. proteins reacted with pooled HME sera (mean OD≥0.2 with background subtracted). All E. ch. proteins ranked according to the immunoreactivity (from high to low) are listed in Table ZS1, and FIG. 7A identifies 40 E. ch. proteins that exhibited the highest immunoreactivity (mean OD>0.8) with pooled sera. The 314 IVTT-expressed E. ca. proteins were similarly screened for immunoreactivity by ELISA with pooled CME sera (IFA titer: 1600), and 39 (12%) proteins were immunoreactive (mean OD≥0.2; FIG. 7B). All E. ca. proteins ranked according to the immunoreactivity (from high to low) are listed in Table ZS2. These immunoreactive E. ch. and E. ca. proteins were investigated further to determine consistency and overall immunoreactivity among a panel of HME or CME sera, respectively.

TABLE ZS1
E. ch. protein immunoreactivity ranked by mean ELISA OD values

| | | Mean | |
|---|---|---|---|
| | Ech_tag | ELISA | Antigenicity |
| No. | no.^{a} | OD^{b} | score |
| 1 | 1065 | 1.67 | 0.70 |
| 2 | 0875 | 1.54 | 0.05 |
| 3 | 0678 | 1.48 | 0.54 |
| 4 | 0207 | 1.37 | 0.06 |
| 5 | 0121 | 1.34 | 0.26 |
| 6 | 0129 | 1.27 | 0.16 |
| 7 | 0640 | 1.26 | 0.50 |
| 8 | 0044 | 1.19 | 0.81 |
| 9 | 1038 | 1.19 | 0.61 |
| 10 | 0706 | 1.17 | 0.16 |
| 11 | 0579 | 1.14 | 0.61 |
| 12 | 0040 | 1.12 | 0.83 |
| 13 | 0252 | 1.12 | 0.88 |
| 14 | 0670 | 1.12 | 0.12 |
| 15 | 0947 | 1.11 | 0.14 |
| 16 | 1128 | 1.10 | 0.44 |
| 17 | 0397 | 1.10 | 0.68 |
| 18 | 0250 | 1.10 | 0.63 |
| 19 | 0673 | 1.08 | 0.14 |
| 20 | 0681 | 1.06 | 0.22 |
| 21 | 0755 | 1.04 | 0.63 |
| 22 | 0883 | 1.02 | 0.62 |
| 23 | 1055 | 1.00 | 0.76 |
| 24 | 0518 | 0.98 | 0.05 |
| 25 | 0665 | 0.98 | 0.70 |
| 26 | 0277 | 0.96 | 0.75 |
| 27 | 0176 | 0.94 | 0.63 |
| 28 | 0304 | 0.93 | 0.66 |
| 29 | 0720 | 0.91 | 0.42 |
| 30 | 0116 | 0.91 | 0.41 |
| 31 | 0089 | 0.90 | 0.62 |
| 32 | 0787 | 0.90 | 0.83 |
| 33 | 0635 | 0.89 | 0.53 |
| 34 | 0395 | 0.87 | 0.67 |
| 35 | 0988 | 0.87 | 0.44 |
| 36 | 0332 | 0.87 | 0.72 |
| 37 | 0520 | 0.86 | 0.77 |
| 38 | 0625 | 0.85 | 0.36 |
| 39 | 04815 | 0.83 | 0.75 |
| 40 | 1057 | 0.80 | 0.71 |
| 41 | 0622 | 0.77 | 0.81 |
| 42 | 0282 | 0.76 | 0.27 |
| 43 | 0021 | 0.75 | 0.20 |
| 44 | 0943 | 0.73 | 0.57 |
| 45 | 0907 | 0.73 | 0.65 |
| 46 | 0128 | 0.71 | 0.45 |
| 47 | 0585 | 0.70 | 0.55 |
| 48 | 0570 | 0.69 | 0.46 |
| 49 | 01930 | 0.65 | 0.10 |
| 50 | 0707 | 0.65 | 0.49 |
| 51 | 0976 | 0.65 | 0.77 |
| 52 | 04985 | 0.65 | 0.72 |
| 53 | 0888 | 0.63 | 0.45 |
| 54 | 0445 | 0.62 | 0.69 |
| 55 | 0568 | 0.61 | 0.42 |
| 56 | 0167 | 0.61 | 0.65 |
| 57 | 0155 | 0.60 | 0.66 |
| 58 | 0033 | 0.58 | 0.77 |
| 59 | 0710 | 0.58 | 0.45 |
| 60 | 0076 | 0.58 | 0.73 |
| 61 | 1024 | 0.56 | 0.66 |
| 62 | 0768 | 0.53 | 0.62 |
| 63 | 0329 | 0.53 | 0.53 |
| 64 | 0505 | 0.53 | 0.62 |
| 65 | 0275 | 0.52 | 0.17 |
| 66 | 0293 | 0.52 | 0.62 |
| 67 | 1071 | 0.51 | 0.63 |
| 68 | 0335 | 0.50 | 0.65 |
| 69 | 1008 | 0.49 | 0.64 |
| 70 | 0270 | 0.48 | 0.20 |
| 71 | 0235 | 0.47 | 0.68 |
| 72 | 0272 | 0.47 | 0.54 |
| 73 | 0355 | 0.47 | 0.79 |
| 74 | 0049 | 0.43 | 0.13 |
| 75 | 0574 | 0.43 | 0.24 |
| 76 | 1083 | 0.43 | 0.94 |
| 77 | 0189 | 0.43 | 0.88 |
| 78 | 0130 | 0.42 | 0.09 |
| 79 | 0499 | 0.40 | 0.84 |
| 80 | 1041 | 0.40 | 0.51 |
| 81 | 0848 | 0.38 | 0.74 |
| 82 | 0219 | 0.38 | 0.87 |
| 83 | 1059 | 0.38 | 0.40 |
| 84 | 0450 | 0.38 | 0.45 |
| 85 | 0965 | 0.36 | 0.22 |
| 86 | 0916 | 0.34 | 0.56 |
| 87 | 0886 | 0.34 | 0.61 |
| 88 | 0379 | 0.34 | 0.09 |
| 89 | 0500 | 0.33 | 0.68 |
| 90 | 0171 | 0.32 | 0.74 |
| 91 | 0157 | 0.32 | 0.77 |
| 92 | 0486 | 0.32 | 0.67 |
| 93 | 1104 | 0.31 | 0.48 |
| 94 | 0052 | 0.31 | 0.17 |
| 95 | 0682 | 0.31 | 0.03 |
| 96 | 0136 | 0.30 | 0.83 |
| 97 | 0443 | 0.28 | 0.61 |
| 98 | 0985 | 0.28 | 0.65 |
| 99 | 0014 | 0.28 | 0.66 |
| 100 | 0503 | 0.27 | 0.78 |
| 101 | 0591 | 0.27 | 0.81 |
| 102 | 0327 | 0.26 | 0.87 |
| 103 | 0023 | 0.26 | 0.71 |
| 104 | 1011 | 0.26 | 0.68 |
| 105 | 0970 | 0.26 | 0.66 |
| 106 | 0117 | 0.26 | 0.65 |
| 107 | 0849 | 0.24 | 0.56 |
| 108 | 0317 | 0.23 | 0.79 |
| 109 | 0043 | 0.23 | 0.80 |
| 110 | 0989 | 0.22 | 0.35 |
| 111 | 1001 | 0.22 | 0.72 |
| 112 | 1058 | 0.21 | 0.65 |
| 113 | 0057 | 0.21 | 0.60 |
| 114 | 0158 | 0.21 | 0.64 |
| 115 | 1050 | 0.21 | 0.80 |
| 116 | 0750 | 0.21 | 0.64 |
| 117 | 0731 | 0.20 | 0.86 |
| 118 | 1118 | 0.20 | 0.85 |
| 119 | 0278 | 0.19 | 0.35 |
| 120 | 0470 | 0.18 | 0.78 |
| 121 | 1018 | 0.18 | 0.77 |
| 122 | 0871 | 0.18 | 0.76 |
| 123 | 0292 | 0.18 | 0.64 |
| 124 | 0929 | 0.17 | 0.84 |
| 125 | 0835 | 0.17 | 0.66 |
| 126 | 0914 | 0.16 | 0.69 |
| 127 | 0454 | 0.16 | 0.54 |
| 128 | 0797 | 0.16 | 0.70 |
| 129 | 0758 | 0.16 | 0.63 |
| 130 | 0981 | 0.15 | 0.61 |
| 131 | 1020 | 0.15 | 0.73 |
| 132 | 0042 | 0.14 | 0.93 |
| 133 | 0444 | 0.14 | 0.80 |
| 134 | 0010 | 0.14 | 0.14 |
| 135 | 0627 | 0.14 | 0.68 |
| 136 | 0143 | 0.14 | 0.72 |
| 137 | 0699 | 0.13 | 0.47 |
| 138 | 0451 | 0.13 | 0.62 |
| 139 | 0628 | 0.13 | 0.65 |
| 140 | 0854 | 0.13 | 0.53 |
| 141 | 0987 | 0.13 | 0.22 |
| 142 | 0220 | 0.13 | 0.81 |
| 143 | 0353 | 0.13 | 0.35 |
| 144 | 0730 | 0.12 | 0.89 |
| 145 | 0296 | 0.12 | 0.84 |
| 146 | 1073 | 0.12 | 0.91 |
| 147 | 1089 | 0.12 | 0.75 |
| 148 | 0822 | 0.12 | 0.77 |
| 149 | 1031 | 0.12 | 0.72 |
| 150 | 0559 | 0.11 | 0.74 |
| 151 | 0351 | 0.11 | 0.80 |
| 152 | 0185 | 0.11 | 0.70 |
| 153 | 0352 | 0.11 | 0.70 |
| 154 | 0213 | 0.11 | 0.89 |
| 155 | 0364 | 0.11 | 0.70 |
| 156 | 1034 | 0.11 | 0.61 |
| 157 | 1045 | 0.10 | 0.06 |
| 158 | 0378 | 0.10 | 0.61 |
| 159 | 0203 | 0.10 | 0.53 |
| 160 | 0431 | 0.10 | 0.87 |
| 161 | 0652 | 0.09 | 0.64 |
| 162 | 0156 | 0.09 | 0.85 |
| 163 | 0734 | 0.09 | 0.76 |
| 164 | 0630 | 0.09 | 0.76 |
| 165 | 0074 | 0.09 | 0.79 |
| 166 | 0488 | 0.09 | 0.12 |
| 167 | 0691 | 0.09 | 0.74 |
| 168 | 0721 | 0.09 | 0.73 |
| 169 | 0375 | 0.09 | 0.60 |
| 170 | 04990 | 0.09 | 0.15 |
| 171 | 0661 | 0.08 | 0.64 |
| 172 | 0796 | 0.08 | 0.85 |
| 173 | 0615 | 0.08 | 0.89 |
| 174 | 0906 | 0.08 | 0.68 |
| 175 | 0233 | 0.07 | 0.89 |
| 176 | 0025 | 0.07 | 0.88 |
| 177 | 1074 | 0.07 | 0.68 |
| 178 | 0784 | 0.07 | 0.63 |
| 179 | 0301 | 0.07 | 0.82 |
| 180 | 0798 | 0.07 | 0.71 |
| 181 | 0896 | 0.07 | 0.78 |
| 182 | 0587 | 0.07 | 0.37 |
| 183 | 0884 | 0.07 | 0.67 |
| 184 | 0248 | 0.07 | 0.20 |
| 185 | 0263 | 0.07 | 0.91 |
| 186 | 0676 | 0.07 | 0.79 |
| 187 | 0489 | 0.07 | 0.59 |
| 188 | 0717 | 0.06 | 0.05 |
| 189 | 0196 | 0.06 | 0.65 |
| 190 | 0813 | 0.06 | 0.67 |
| 191 | 0802 | 0.06 | 0.68 |
| 192 | 1033 | 0.06 | 0.81 |
| 193 | 1109 | 0.06 | 0.69 |
| 194 | 0662 | 0.06 | 0.56 |
| 195 | 0432 | 0.06 | 0.68 |
| 196 | 0412 | 0.06 | 0.79 |
| 197 | 0582 | 0.06 | 0.19 |
| 198 | 0184 | 0.05 | 0.63 |
| 199 | 0830 | 0.05 | 0.86 |
| 200 | 1014 | 0.05 | 0.68 |
| 201 | 0539 | 0.05 | 0.71 |
| 202 | 0490 | 0.05 | 0.74 |
| 203 | 02505 | 0.05 | 0.78 |
| 204 | 1114 | 0.05 | 0.77 |
| 205 | 0038 | 0.05 | 0.79 |
| 206 | 0098 | 0.05 | 0.62 |
| 207 | 0287 | 0.05 | 0.32 |
| 208 | 0374 | 0.05 | 0.81 |
| 209 | 0692 | 0.04 | 0.38 |
| 210 | 0218 | 0.04 | 0.83 |
| 211 | 0487 | 0.04 | 0.73 |
| 212 | 0815 | 0.04 | 0.95 |
| 213 | 0633 | 0.04 | 0.72 |
| 214 | 0649 | 0.04 | 0.70 |
| 215 | 1052 | 0.04 | 0.61 |
| 216 | 0232 | 0.04 | 0.70 |
| 217 | 0650 | 0.04 | 0.72 |
| 218 | 0485 | 0.04 | 0.64 |
| 219 | 0442 | 0.04 | 0.81 |
| 220 | 0983 | 0.04 | 0.72 |
| 221 | 0980 | 0.04 | 0.84 |
| 222 | 0946 | 0.04 | 0.68 |
| 223 | 1100 | 0.03 | 0.16 |
| 224 | 0316 | 0.03 | 0.65 |
| 225 | 1066 | 0.03 | 0.84 |
| 226 | 0931 | 0.03 | 0.81 |
| 227 | 0881 | 0.03 | 0.72 |
| 228 | 0760 | 0.03 | 0.73 |
| 229 | 0078 | 0.03 | 0.62 |
| 230 | 0695 | 0.03 | 0.86 |
| 231 | 0471 | 0.03 | 0.84 |
| 232 | 0664 | 0.03 | 0.07 |
| 233 | 0631 | 0.03 | 0.80 |
| 234 | 0632 | 0.03 | 0.83 |
| 235 | 0639 | 0.03 | 0.44 |
| 236 | 0584 | 0.03 | 0.80 |
| 237 | 0309 | 0.03 | 0.74 |
| 238 | 0785 | 0.03 | 0.66 |
| 239 | 0396 | 0.03 | 0.43 |
| 240 | 0002 | 0.03 | 0.79 |
| 241 | 0979 | 0.03 | 0.61 |
| 242 | 0853 | 0.03 | 0.74 |
| 243 | 0756 | 0.03 | 0.88 |
| 244 | 0654 | 0.02 | 0.70 |
| 245 | 04700 | 0.02 | 0.75 |
| 246 | 0011 | 0.02 | 0.74 |
| 247 | 0563 | 0.02 | 0.87 |
| 248 | 0705 | 0.02 | 0.88 |
| 249 | 0152 | 0.02 | 0.75 |
| 250 | 1054 | 0.02 | 0.80 |
| 251 | 0493 | 0.02 | 0.86 |
| 252 | 0154 | 0.02 | 0.72 |
| 253 | 1025 | 0.02 | 0.91 |
| 254 | 0619 | 0.02 | 0.47 |
| 255 | 02925 | 0.02 | 0.73 |
| 256 | 0391 | 0.02 | 0.62 |
| 257 | 0753 | 0.02 | 0.28 |
| 258 | 0689 | 0.02 | 0.79 |
| 259 | 0837 | 0.02 | 0.87 |
| 260 | 0777 | 0.02 | 0.89 |
| 261 | 0764 | 0.02 | 0.40 |
| 262 | 0577 | 0.02 | 0.60 |
| 263 | 0475 | 0.02 | 0.66 |
| 264 | 0302 | 0.02 | 0.64 |
| 265 | 0359 | 0.02 | 0.60 |
| 266 | 0597 | 0.02 | 0.70 |
| 267 | 0826 | 0.02 | 0.90 |
| 268 | 0939 | 0.02 | 0.79 |
| 269 | 1111 | 0.02 | 0.87 |
| 270 | 0533 | 0.01 | 0.64 |
| 271 | 0109 | 0.01 | 0.50 |
| 272 | 0239 | 0.01 | 0.61 |
| 273 | 0168 | 0.01 | 0.85 |
| 274 | 0389 | 0.01 | 0.72 |
| 275 | 1026 | 0.01 | 0.62 |
| 276 | 0105 | 0.01 | 0.09 |
| 277 | 0501 | 0.01 | 0.83 |
| 278 | 0795 | 0.01 | 0.65 |
| 279 | 0399 | 0.01 | 0.61 |
| 280 | 0303 | 0.01 | 0.74 |
| 281 | 0366 | 0.01 | 0.63 |
| 282 | 0956 | 0.01 | 0.85 |
| 283 | 0102 | 0.01 | 0.19 |
| 284 | 0735 | 0.01 | 0.92 |
| 285 | 0326 | 0.01 | 0.79 |
| 286 | 0759 | 0.01 | 0.04 |
| 287 | 0834 | 0.01 | 0.47 |
| 288 | 0204 | 0.01 | 0.89 |
| 289 | 0937 | 0.01 | 0.64 |
| 290 | 1029 | 0.01 | 0.61 |
| 291 | 0050 | 0.01 | 0.61 |
| 292 | 1075 | 0.01 | 0.62 |
| 293 | 0992 | 0.01 | 0.82 |
| 294 | 0634 | 0 | 0.62 |
| 295 | 0368 | 0 | 0.71 |
| 296 | 0012 | 0 | 0.62 |
| 297 | 0354 | 0 | 0.69 |
| 298 | 0659 | 0 | 0.21 |
| 299 | 0312 | 0 | 0.76 |
| 300 | 0343 | 0 | 0.50 |
| 301 | 0103 | 0 | 0.15 |
| 302 | 0140 | 0 | 0.65 |
| 303 | 0144 | 0 | 0.84 |
| 304 | 1153 | 0 | 0.78 |
| 305 | 0320 | 0 | 0.60 |
| 306 | 0895 | 0 | 0.63 |
| 307 | 0669 | 0 | 0.76 |
| 308 | 1030 | 0 | 0.18 |
| 309 | 0107 | 0 | 0.69 |
| 310 | 0016 | 0 | 0.75 |
| 311 | 0064 | 0 | 0.80 |
| 312 | 0188 | 0 | 0.86 |
| 313 | 0041 | 0 | 0.86 |
| 314 | 0201 | 0 | 0.74 |
| 315 | 0370 | 0 | 0.65 |
| 316 | 0267 | 0 | 0.81 |
| 317 | 0748 | 0 | 0.67 |
| 318 | 0997 | 0 | 0.64 |
| 319 | 0697 | 0 | 0.54 |
| 320 | 0498 | 0 | 0.94 |

^{a}Tag no. with 5 digits is from NCBI.
^{b}Mean OD from pooled HME patient sera.

TABLE ZS2
E. ca. protein immunoreactivity ranked by mean ELISA OD values

| | | Mean | |
|---|---|---|---|
| | Ecaj_tag | ELISA | Antigenicity |
| No. | no.^{a} | OD^{b} | score |
| 1 | 0917 | 1.96 | 0.82 |
| 2 | 0151 | 1.89 | 0.85 |
| 3 | 0162 | 1.88 | 0.60 |
| 4 | 0916 | 1.87 | 0.81 |
| 5 | 0805 | 1.80 | 0.75 |
| 6 | 0094 | 1.80 | 0.78 |
| 7 | 0857 | 1.77 | 0.75 |
| 8 | 0128 | 1.77 | 0.90 |
| 9 | 0179 | 1.71 | 0.77 |
| 10 | 0213 | 1.66 | 0.61 |
| 11 | 0334 | 1.61 | 0.87 |
| 12 | 0589 | 1.60 | 0.80 |
| 13 | 0535 | 1.56 | 0.86 |
| 14 | 0554 | 1.34 | 0.84 |
| 15 | 0333 | 1.29 | 0.62 |
| 16 | 0818 | 1.27 | 0.73 |
| 17 | 0850 | 1.22 | 0.71 |
| 18 | 0849 | 1.22 | 0.77 |
| 19 | 0332 | 1.17 | 0.72 |
| 20 | 0851 | 1.14 | 0.24 |
| 21 | 0728 | 1.07 | 0.50 |
| 22 | 0161 | 1.06 | 0.60 |
| 23 | 0104 | 1.02 | 0.39 |
| 24 | 0321 | 0.99 | 0.29 |
| 25 | 0637 | 0.80 | 0.61 |
| 26 | 0915 | 0.80 | 0.78 |
| 27 | 0787 | 0.79 | 0.80 |
| 28 | 0472 | 0.75 | 0.49 |
| 29 | 0737 | 0.62 | 0.56 |
| 30 | 0747 | 0.54 | 0.49 |
| 31 | 0467 | 0.53 | 0.78 |
| 32 | 0738 | 0.39 | 0.59 |
| 33 | 0622 | 0.34 | 0.10 |
| 34 | 0671 | 0.34 | 0.86 |
| 35 | 0882 | 0.32 | 0.19 |
| 36 | 0648 | 0.31 | 0.84 |
| 37 | 0938 | 0.30 | 0.07 |
| 38 | 0372 | 0.21 | 0.12 |
| 39 | 0746 | 0.21 | 0.49 |
| 40 | 0863 | 0.19 | 0.66 |
| 41 | 0454 | 0.16 | 0.75 |
| 42 | 0664 | 0.16 | 0.56 |
| 43 | 0894 | 0.14 | 0.74 |
| 44 | 0906 | 0.14 | 0.78 |
| 45 | 0749 | 0.12 | 0.21 |
| 46 | 0905 | 0.11 | 0.75 |
| 47 | 0740 | 0.11 | 0.66 |
| 48 | 0448 | 0.11 | 0.76 |
| 49 | 0724 | 0.11 | 0.42 |
| 50 | 0433 | 0.11 | 0.15 |
| 51 | 0366 | 0.10 | 0.07 |
| 52 | 0416 | 0.10 | 0.18 |
| 53 | 0110 | 0.10 | 0.77 |
| 54 | 0896 | 0.10 | 0.77 |
| 55 | 0349 | 0.09 | 0.06 |
| 56 | 05065 | 0.09 | 0.17 |
| 57 | 0538 | 0.09 | 0.62 |
| 58 | 0623 | 0.09 | 0.75 |
| 59 | 0045 | 0.09 | 0.73 |
| 60 | 0796 | 0.08 | 0.04 |
| 61 | 05025 | 0.08 | 0.12 |
| 62 | 0918 | 0.08 | 0.74 |
| 63 | 0551 | 0.08 | 0.64 |
| 64 | 0848 | 0.08 | 0.85 |
| 65 | 0788 | 0.08 | 0.69 |
| 66 | 0002 | 0.08 | 0.04 |
| 67 | 0358 | 0.07 | 0.50 |
| 68 | 0141 | 0.07 | 0.54 |
| 69 | 0172 | 0.07 | 0.09 |
| 70 | 0837 | 0.07 | 0.36 |
| 71 | 0786 | 0.07 | 0.80 |
| 72 | 0191 | 0.07 | 0.69 |
| 73 | 0545 | 0.07 | 0.63 |
| 74 | 0897 | 0.07 | 0.16 |
| 75 | 0331 | 0.07 | 0.91 |
| 76 | 0288 | 0.07 | 0.84 |
| 77 | 0456 | 0.07 | 0.44 |
| 78 | 0480 | 0.07 | 0.65 |
| 79 | 0902 | 0.07 | 0.60 |
| 80 | 0673 | 0.07 | 0.90 |
| 81 | 0248 | 0.06 | 0.82 |
| 82 | 0373 | 0.06 | 0.12 |
| 83 | 0079 | 0.06 | 0.54 |
| 84 | 0155 | 0.06 | 0.75 |
| 85 | 0802 | 0.06 | 0.68 |
| 86 | 0419 | 0.06 | 0.79 |
| 87 | 0081 | 0.06 | 0.09 |
| 88 | 0369 | 0.06 | 0.51 |
| 89 | 0210 | 0.06 | 0.37 |
| 90 | 0770 | 0.06 | 0.25 |
| 91 | 0507 | 0.06 | 0.66 |
| 92 | 0233 | 0.06 | 0.60 |
| 93 | 0217 | 0.06 | 0.70 |
| 94 | 0091 | 0.06 | 0.82 |
| 95 | 0315 | 0.05 | 0.76 |
| 96 | 0335 | 0.05 | 0.94 |
| 97 | 0399 | 0.05 | 0.55 |
| 98 | 05005 | 0.05 | 0.06 |
| 99 | 0856 | 0.05 | 0.75 |
| 100 | 0904 | 0.05 | 0.60 |
| 101 | 0732 | 0.05 | 0.28 |
| 102 | 0484 | 0.05 | 0.29 |
| 103 | 0845 | 0.05 | 0.77 |
| 104 | 0578 | 0.05 | 0.78 |
| 105 | 0721 | 0.05 | 0.59 |
| 106 | 0088 | 0.05 | 0.73 |
| 107 | 0290 | 0.05 | 0.76 |
| 108 | 0338 | 0.05 | 0.69 |
| 109 | 0447 | 0.05 | 0.68 |
| 110 | 0843 | 0.05 | 0.81 |
| 111 | 0390 | 0.05 | 0.72 |
| 112 | 0279 | 0.05 | 0.80 |
| 113 | 0755 | 0.05 | 0.16 |
| 114 | 0702 | 0.05 | 0.87 |
| 115 | 0829 | 0.05 | 0.87 |
| 116 | 0278 | 0.05 | 0.75 |
| 117 | 0502 | 0.05 | 0.59 |
| 118 | 0833 | 0.05 | 0.82 |
| 119 | 0425 | 0.04 | 0.22 |
| 120 | 0463 | 0.04 | 0.66 |
| 121 | 0721 | 0.04 | 0.59 |
| 122 | 0513 | 0.04 | 0.07 |
| 123 | 0643 | 0.04 | 0.81 |
| 124 | 0124 | 0.04 | 0.81 |
| 125 | 0890 | 0.04 | 0.77 |
| 126 | 0678 | 0.04 | 0.92 |
| 127 | 0731 | 0.04 | 0.68 |
| 128 | 0718 | 0.04 | 0.17 |
| 129 | 0509 | 0.04 | 0.60 |
| 130 | 03785 | 0.04 | 0.65 |
| 131 | 05040 | 0.04 | 0.07 |
| 132 | 0568 | 0.04 | 0.56 |
| 133 | 0773 | 0.04 | 0.74 |
| 134 | 0750 | 0.04 | 0.71 |
| 135 | 0268 | 0.04 | 0.59 |
| 136 | 0627 | 0.04 | 0.82 |
| 137 | 0555 | 0.04 | 0.81 |
| 138 | 0494 | 0.04 | 0.75 |
| 139 | 0744 | 0.03 | 0.50 |
| 140 | 0675 | 0.03 | 0.78 |
| 141 | 0766 | 0.03 | 0.23 |
| 142 | 0225 | 0.03 | 0.66 |
| 143 | 0020 | 0.03 | 0.94 |
| 144 | 0201 | 0.03 | 0.73 |
| 145 | 0411 | 0.03 | 0.60 |
| 146 | 0655 | 0.03 | 0.82 |
| 147 | 0363 | 0.03 | 0.79 |
| 148 | 0080 | 0.03 | 0.26 |
| 149 | 0308 | 0.03 | 0.66 |
| 150 | 0496 | 0.03 | 0.49 |
| 151 | 0361 | 0.03 | 0.81 |
| 152 | 0070 | 0.03 | 0.50 |
| 153 | 0754 | 0.03 | 0.18 |
| 154 | 0859 | 0.03 | 0.64 |
| 155 | 0808 | 0.03 | 0.69 |
| 156 | 0577 | 0.03 | 0.73 |
| 157 | 0351 | 0.03 | 0.82 |
| 158 | 0064 | 0.03 | 0.42 |
| 159 | 0240 | 0.03 | 0.68 |
| 160 | 0406 | 0.03 | 0.73 |
| 161 | 0714 | 0.03 | 0.25 |
| 162 | 0485 | 0.03 | 0.15 |
| 163 | 0634 | 0.03 | 0.43 |
| 164 | 0415 | 0.03 | 0.28 |
| 165 | 0831 | 0.03 | 0.72 |
| 166 | 0777 | 0.03 | 0.93 |
| 167 | 0752 | 0.02 | 0.37 |
| 168 | 0705 | 0.02 | 0.69 |
| 169 | 0758 | 0.02 | 0.86 |
| 170 | 0280 | 0.02 | 0.88 |
| 171 | 0291 | 0.02 | 0.67 |
| 172 | 0368 | 0.02 | 0.06 |
| 173 | 0027 | 0.02 | 0.39 |
| 174 | 0252 | 0.02 | 0.82 |
| 175 | 0638 | 0.02 | 0.70 |
| 176 | 0719 | 0.02 | 0.49 |
| 177 | 0539 | 0.02 | 0.59 |
| 178 | 0733 | 0.02 | 0.36 |
| 179 | 0090 | 0.02 | 0.69 |
| 180 | 0734 | 0.02 | 0.42 |
| 181 | 0114 | 0.02 | 0.86 |
| 182 | 0219 | 0.02 | 0.64 |
| 183 | 0925 | 0.02 | 0.79 |
| 184 | 0483 | 0.02 | 0.24 |
| 185 | 0865 | 0.02 | 0.85 |
| 186 | 0707 | 0.02 | 0.59 |
| 187 | 0355 | 0.02 | 0.51 |
| 188 | 0412 | 0.02 | 0.67 |
| 189 | 0435 | 0.02 | 0.41 |
| 190 | 05060 | 0.02 | 0.76 |
| 191 | 0471 | 0.02 | 0.67 |
| 192 | 04995 | 0.02 | 0.31 |
| 193 | 05055 | 0.02 | 0.24 |
| 194 | 0140 | 0.01 | 0.33 |
| 195 | 0827 | 0.01 | 0.85 |
| 196 | 0388 | 0.01 | 0.62 |
| 197 | 0479 | 0.01 | 0.42 |
| 198 | 0394 | 0.01 | 0.63 |
| 199 | 0687 | 0.01 | 0.70 |
| 200 | 04520 | 0.01 | 0.78 |
| 201 | 0100 | 0.01 | 0.89 |
| 202 | 0025 | 0.01 | 0.68 |
| 203 | 0344 | 0.01 | 0.63 |
| 204 | 0285 | 0.01 | 0.75 |
| 205 | 01340 | 0.01 | 0.05 |
| 206 | 0136 | 0.01 | 0.83 |
| 207 | 0761 | 0.01 | 0.85 |
| 208 | 0512 | 0.01 | 0.89 |
| 209 | 0442 | 0.01 | 0.73 |
| 210 | 0847 | 0.01 | 0.74 |
| 211 | 0136 | 0.01 | 0.83 |
| 212 | 0377 | 0.01 | 0.66 |
| 213 | 0404 | 0.01 | 0.53 |
| 214 | 0572 | 0.01 | 0.45 |
| 215 | 0436 | 0.01 | 0.41 |
| 216 | 0653 | 0 | 0.68 |
| 217 | 04965 | 0 | 0.81 |
| 218 | 0111 | 0 | 0.91 |
| 219 | 0054 | 0 | 0.64 |
| 220 | 0823 | 0 | 0.64 |
| 221 | 0547 | 0 | 0.59 |
| 222 | 0282 | 0 | 0.69 |
| 223 | 0804 | 0 | 0.71 |
| 224 | 0769 | 0 | 0.08 |
| 225 | 04970 | 0 | 0.81 |
| 226 | 0839 | 0 | 0.44 |
| 227 | 0509 | 0 | 0.60 |
| 228 | 0783 | 0 | 0.70 |
| 229 | 0144 | 0 | 0.68 |
| 230 | 0768 | 0 | 0.50 |
| 231 | 0525 | 0 | 0.90 |
| 232 | 0735 | 0 | 0.24 |
| 233 | 0206 | 0 | 0.86 |
| 234 | 0414 | 0 | 0.31 |
| 235 | 03735 | 0 | 0.43 |
| 236 | 0138 | 0 | 0.71 |
| 237 | 0866 | 0 | 0.83 |
| 238 | 0357 | 0 | 0.54 |
| 239 | 0775 | 0 | 0.71 |
| 240 | 0711 | 0 | 0.49 |
| 241 | 0173 | 0 | 0.70 |
| 242 | 02575 | 0 | 0.76 |
| 243 | 0408 | 0 | 0.75 |
| 244 | 0537 | 0 | 0.71 |
| 245 | 0409 | 0 | 0.88 |
| 246 | 0656 | 0 | 0.67 |
| 247 | 0350 | 0 | 0.89 |
| 248 | 0756 | 0 | 0.57 |
| 249 | 0742 | 0 | 0.69 |
| 250 | 0460 | 0 | 0.19 |
| 251 | 0799 | 0 | 0.56 |
| 252 | 0044 | 0 | 0.67 |
| 253 | 0391 | 0 | 0.80 |
| 254 | 05030 | 0 | 0.43 |
| 255 | 0183 | 0 | 0.66 |
| 256 | 0646 | 0 | 0.70 |
| 257 | 0481 | 0 | 0.45 |
| 258 | 0147 | 0 | 0.71 |
| 259 | 0103 | 0 | 0.66 |
| 260 | 0571 | 0 | 0.63 |
| 261 | 0133 | 0 | 0.65 |
| 262 | 0489 | 0 | 0.82 |
| 263 | 0639 | 0 | 0.63 |
| 264 | 0657 | 0 | 0.67 |
| 265 | 0527 | 0 | 0.82 |
| 266 | 0256 | 0 | 0.84 |
| 267 | 0385 | 0 | 0.67 |
| 268 | 0323 | 0 | 0.64 |
| 269 | 0192 | 0 | 0.68 |
| 270 | 0887 | 0 | 0.70 |
| 271 | 0828 | 0 | 0.64 |
| 272 | 0564 | 0 | 0.68 |
| 273 | 0506 | 0 | 0.60 |
| 274 | 0380 | 0 | 0.49 |
| 275 | 0226 | 0 | 0.86 |
| 276 | 0102 | 0 | 0.75 |
| 277 | 0580 | 0 | 0.59 |
| 278 | 0427 | 0 | 0.92 |
| 279 | 0751 | 0 | 0.44 |
| 280 | 0446 | 0 | 0.82 |
| 281 | 0143 | 0 | 0.60 |
| 282 | 0945 | 0 | 0.71 |
| 283 | 0420 | 0 | 0.61 |
| 284 | 0870 | 0 | 0.94 |
| 285 | 0311 | 0 | 0.44 |
| 286 | 0679 | 0 | 0.69 |
| 287 | 0266 | 0 | 0.95 |
| 288 | 0633 | 0 | 0.60 |
| 289 | 0258 | 0 | 0.89 |
| 290 | 0199 | 0 | 0.63 |
| 291 | 0615 | 0 | 0.65 |
| 292 | 0180 | 0 | 0.72 |
| 293 | 0628 | 0 | 0.74 |
| 294 | 03365 | 0 | 0.12 |
| 295 | 0523 | 0 | 0.65 |
| 296 | 0753 | 0 | 0.56 |
| 297 | 0318 | 0 | 0.89 |
| 298 | 0791 | 0 | 0.66 |
| 299 | 0400 | 0 | 0.22 |
| 300 | 0889 | 0 | 0.73 |
| 301 | 0700 | 0 | 0.65 |
| 302 | 0374 | 0 | 0.73 |
| 303 | 0858 | 0 | 0.73 |
| 304 | 0273 | 0 | 0.71 |
| 305 | 0826 | 0 | 0.65 |
| 306 | 0641 | 0 | 0.70 |
| 307 | 0729 | 0 | 0.61 |
| 308 | 0683 | 0 | 0.64 |
| 309 | 0819 | 0 | 0.65 |
| 310 | 0830 | 0 | 0.61 |
| 311 | 0444 | 0 | 0.66 |
| 312 | 0237 | 0 | 0.63 |
| 313 | 0722 | 0 | 0.66 |
| 314 | 0383 | 0 | 0.30 |

^{a}Tag no. with 5 digits is from NCBI.
^{b}Mean OD from pooled CME dog sera.

### Identification of Major Immunoreactive E. ch. And E. ca. Proteins

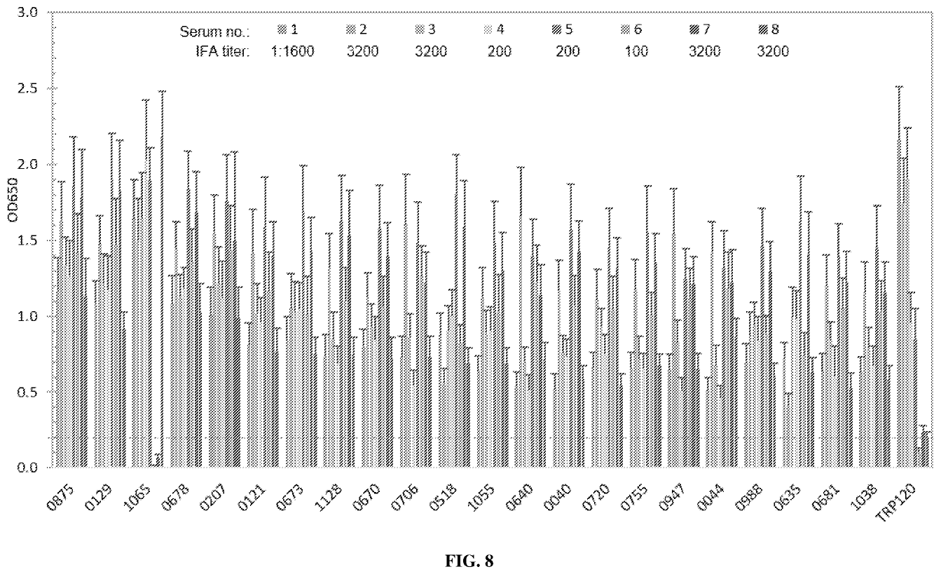

In order to define and compare the immunoreactivity, we used a panel of 8 HME and 10 CME sera to further investigate the immunoreactive E. ch. and E. ca. proteins by ELISA. All the patient and canine sera recognized E. ch. or E. ca. by IFA, and the antibody titers ranged from 100 to 3200. Well-defined major immunoreactive proteins E. ch. TRP120 and E. ca. TRP19 were used as positive controls. Of the 118 E. ch. immunoreactive proteins identified, we selected 40 proteins with strong reactions (OD>0.8) for further reactivity characterization using multiple HME sera. Eighteen (45%) of these immunoreactive proteins were ranked in the top 350 by ANTIGENpro, and 25 proteins (63%) were hypothetical, consistent with our previous finding that more hypothetical proteins were immunoreactive than annotated proteins (21). All these proteins were recognized by eight HME sera, except that Ech_1065 which was recognized by six sera. The top 12 proteins that reacted strongly with all or most HME sera (similar to gold standard TRP120) were considered immunodominant based on mean ELISA OD values (≥1.0). An additional 28 proteins reacted strongly with some HME sera and consistently with all sera, but at lower levels (mean OD<1.0). Thus, these immunoreactive proteins were classified as subdominant. None of the HME sera reacted with the IVTT-expressed negative control protein. The top 22 immunoreactive proteins ranked by mean ELISA OD values (>0.9) are shown in FIG. 8 and Table Z1.

TABLE Z1
Top new E. ch. protein immunoreactivity and ANTIGENpro analysis

| | | | | E.ca. ortholog/ |
|---|---|---|---|---|
| Protein | Mean | ANTIGENpro | Antigenicity | ANTIGENpro |
| (Ech_tag no.) | ELISA OD^{a} | rank | score | rank/Immunoreactive^{b} |
| 0875 | 1.45 | 1039 | 0.06 | 0223/803/na |
| 0129 | 1.38 | 849 | 0.16 | 0080/706/− |
| 1065 | 1.37 | 242 | 0.70 | 0857/194/++ |
| 0678 | 1.34 | 424 | 0.54 | 0369/450/− |
| 0207 | 1.32 | 1013 | 0.06 | 0796/919/− |
| 0121 | 1.14 | 722 | 0.26 | 0071,0072/23,53/+, − |
| 0673 | 1.11 | 876 | 0.14 | 0372/832/− |
| 1128 | 1.07 | 524 | 0.44 | * |
| 0670 | 1.06 | 900 | 0.12 | 0373/835/− |
| 0706 | 1.05 | 840 | 0.16 | 0349/890/− |
| 0518 | 1.03 | 1040 | 0.05 | 0513/877/− |
| 1055 | 1.01 | 181 | 0.76 | 0848/100/− |
| 0640 | 0.99 | 467 | 0.50 | 0399/414/− |
| 0040 | 0.98 | 106 | 0.83 | 0018/130/na |
| 0720 | 0.98 | 559 | 0.42 | 0344/333/− |
| 0755 | 0.98 | 316 | 0.63 | 0319/411/na |
| 0947 | 0.97 | 875 | 0.14 | 0172/861/− |
| 0044 | 0.96 | 122 | 0.81 | 0022/115/na |
| 0988 | 0.95 | 528 | 0.44 | 0141/421/− |
| 0635 | 0.94 | 429 | 0.53 | 0404/431/− |
| 0681 | 0.93 | 771 | 0.22 | 0366/881/− |
| 1038 | 0.93 | 333 | 0.61 | 0838/18/− |

^{a}Mean OD from 8 HME patient sera.
^{b}(++) immunodominant;
(+) immunoreactive in immunoscreening;
(−) not immunoreactive in immunoscreening;
(na) not available;
(*) E.ca. ortholog not identified.

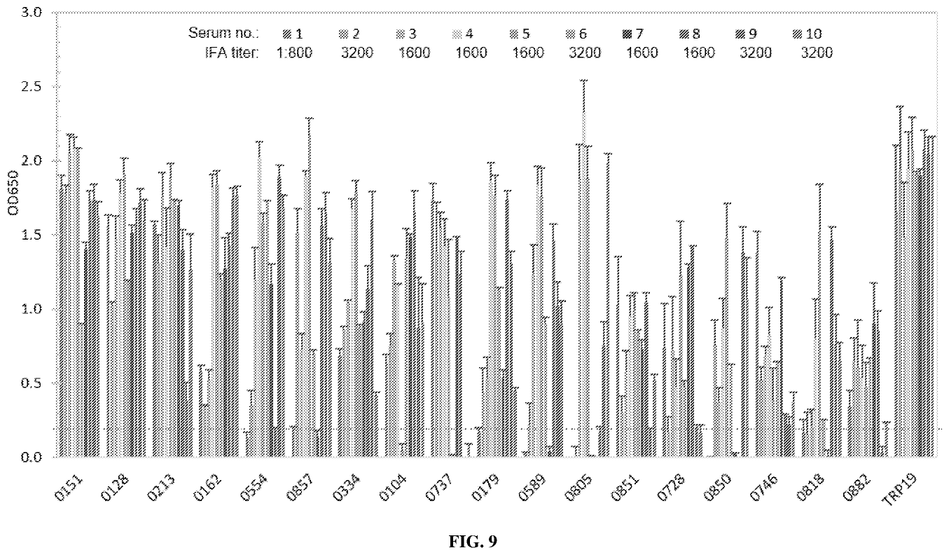

Of the 39 E. ca. immunoreactive proteins identified (OD>0.2), 28 (72%) proteins were ranked in the top 350 by ANTIGENpro, and 12 proteins (31%) were hypothetical. The reactivity of these proteins was further characterized by ELISA with multiple CME sera and all these proteins were recognized by most CME sera. Top nine E. ca. proteins reacted strongly with most canine sera at a level comparable to TRP19 (mean OD>1.0), thus were considered immunodominant. Another 30 E. ca. proteins reacted with a mean ELISA OD values <1.0 and were classified as subdominant. None of the CME sera reacted with the IVTT-expressed negative control protein. Top 18 proteins ranked by mean ELISA OD values (>0.5) are shown in FIG. 9 and Table Z2. Some new immunoreactive proteins of E. ch. and E. ca were outside the ANTIGENpro top 350 list and had low predicted antigenicity score and rank, demonstrating that many hypothetical proteins are potentially immunoreactive despite low antigenicity scores predicted by ANTIGENpro (Table Z1 and Table Z2). Without wishing to be bound by any theory, this might be partially due to the databases and training model that ANTIGENpro uses for machine learning which may have a bias for proteins with known function. These results demonstrate the limitations of predictive software such as ANTIGENpro at anticipating which hypothetical proteins might be antigenic in vivo. This data obtained using ELISA with multiple sera showed that multiple polypeptides including hypothetical proteins exhibited surprising and unexpected antigenicity in vivo.

TABLE Z2
Top new E. ca. protein immunoreactivity and ANTIGENpro analysis

| | | | | E.ch. ortholog/ |
|---|---|---|---|---|
| Protein | Mean | ANTIGENpro | Antigenicity | ANTIGENpro |
| (Ecaj_tag no.) | ELISA OD^{a} | rank | score | rank/Immunoreactive^{b} |
| 0151 | 1.72 | 96 | 0.85 | 0976/161/+ |
| 0128 | 1.53 | 50 | 0.90 | 0189/51/+ |
| 0213 | 1.44 | 348 | 0.61 | * |
| 0162 | 1.25 | 362 | 0.60 | 0960/551/na |
| 0554 | 1.20 | 103 | 0.84 | 0471/91/− |
| 0857 | 1.19 | 194 | 0.75 | 1065/242/++ |
| 0334 | 1.10 | 76 | 0.87 | 0731/77/+ |
| 0104 | 1.02 | 577 | 0.39 | 0159/175/+ |
| 0737 | 1.00 | 397 | 0.56 | * |
| 0179 | 0.99 | 181 | 0.77 | 0939/150/− |
| 0589 | 0.94 | 156 | 0.80 | 0432/258/− |
| 0805 | 0.89 | 193 | 0.75 | 0997/301/− |
| 0851 | 0.72 | 767 | 0.20 | * |
| 0728 | 0.69 | 466 | 0.50 | * |
| 0850 | 0.66 | 244 | 0.71 | 1058/287/+ |
| 0746 | 0.65 | 473 | 0.49 | * |
| 0818 | 0.60 | 213 | 0.73 | * |
| 0882 | 0.53 | 781 | 0.19 | 1104/483/+ |

^{a}Mean OD from 10 CME patient sera.
^{b}(++) immunodominant;
(+) immunoreactive in immunoscreening;
(−) not immunoreactive in immunoscreening;
(na) not available;
(*) E.ch. ortholog not identified.

Antibody Epitopes of E. ch. And E. ca. Immunoreactive Proteins

Immunoreactive proteins of Ehrlichia previously identified by immunoblot contain linear epitopes and are limited in number (4). Recently, we discovered new immunoreactive Ehrlichia proteins that are not detectable by conventional immunoblotting approaches, revealing the dominance of conformation-dependent antibody epitopes in these proteins (21). To further identify new immunoreactive E. ch. and E. ca. proteins in this study, we compared the immunoreactivity by ELISA of native proteins (IVTT products) with that of denatured proteins (IVTT products treated by urea) with the same panel of HME or CME sera.

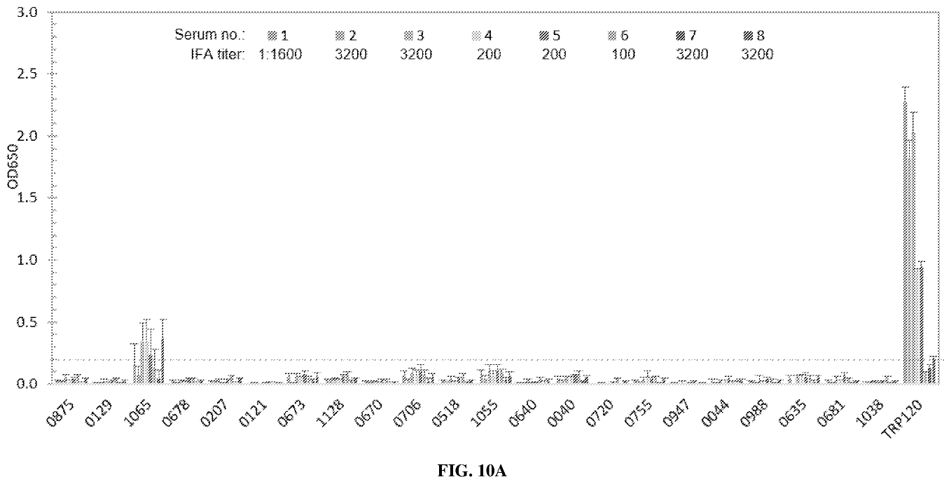

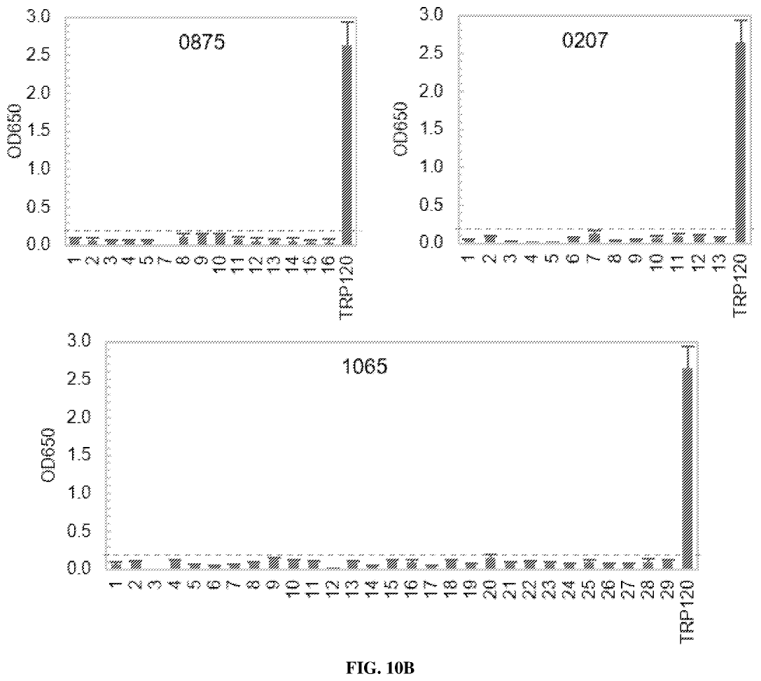

After denaturation, only one E. ch. immunoreactive proteins (Ech_1065) among top 22 still reacted weakly (mean OD=0.21) with four HME sera, compared to its native IVTT protein (mean OD=1.37). All other denatured E. ch. proteins did not react with any HME serum, while the major immunoreactive protein control, TRP120, was affected by denaturation, since it contains a major linear epitope (FIG. 10A) (4). These results indicate that the new E. ch. immunoreactive proteins are defined by conformation-dependent antibody epitopes.

In order to define if new E. ch. immunoreactive proteins contain linear epitopes, we used synthetic peptides to map linear epitopes in these proteins (5, 6, 9). Overlapping polypeptides (19-20 amino acids; 6 amino acid overlap) were synthesized to cover the sequence of three selected E. ch. immunoreactive proteins (Ech_0207, 0875 and 1065), except that peptide 6 of Ech_0875 was not synthesized successfully due to its strong hydrophobicity. The pooled HME sera used in our initial screening was used to probe all peptides by ELISA (FIG. 10B). None of these peptides reacted with HME sera, supporting the conclusion that these E. ch. immunoreactive proteins do not contain major linear epitopes, a finding consistent with ELISA using native and denatured IVTT products (FIGS. 8 and 10A).

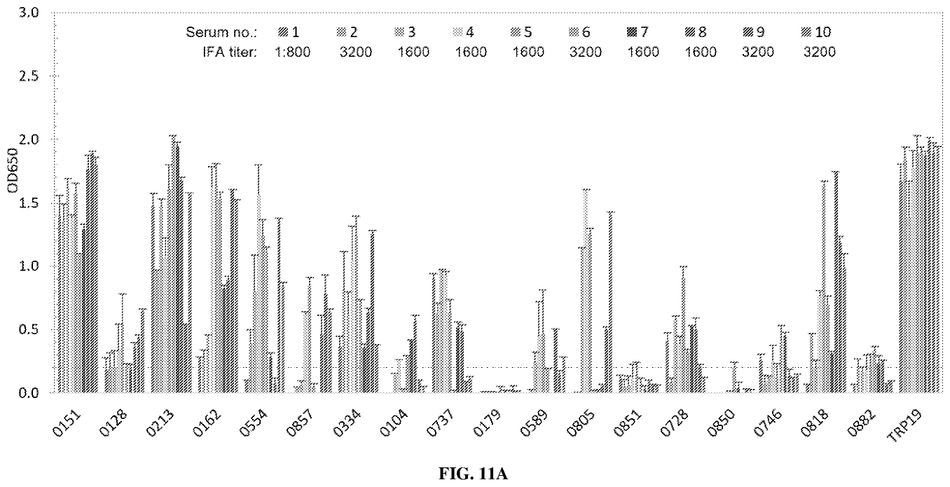

Among the top 18 new E. ca. immunoreactive proteins, the immunoreactivity of five proteins (Ecaj_0151, 0213, 0162, 0818 and 0563) was not reduced substantially after denaturation, similar to well-defined E. ca. major immunoreactive protein TRP19 that has defined linear antibody epitope; however, three proteins, including Ecaj_0179, 0851, and 0850, did not react with any canine serum. An additional 10 E. ca. proteins reacted with canine sera at a substantially lower level compared to native IVTT proteins (FIG. 11A). Thus, our results indicate that 13 of 18 new E. ca. immunoreactive proteins have conformation-dependent antibody epitopes.

We also selected three E. ca. proteins (Ecaj_0213, 0104 and 0737) to investigate conformational dependence using overlapping synthetic peptides. These three proteins were predicted to contain different types of antibody epitope according to our ELISA results. By ELISA, peptide 1 of Ecaj_0213 reacted strongly with pooled CME sera and four peptides of Ecaj_0213 (peptides 2, 10, 22 and 23) reacted with pooled CME sera, suggesting the presence of a major linear epitope and a few minor linear epitopes in this protein. Other peptides of Ecaj_0213 did not react with canine sera except that peptide 8 was not synthesized successfully due to its strong hydrophobicity. Three peptides of Ecaj_0737 (peptides 6, 12 and 13) reacted with pooled CME sera, but at a substantially lower level than the whole protein, suggesting the presence of a few minor linear epitopes. None of the Ecaj_0104 peptides reacted with CME sera, suggesting the absence of linear epitopes. These results support the conclusion that some new E. ca. immunoreactive proteins contain major conformational epitopes while some others contain linear epitopes or both (FIG. 9 and FIG. 11A).

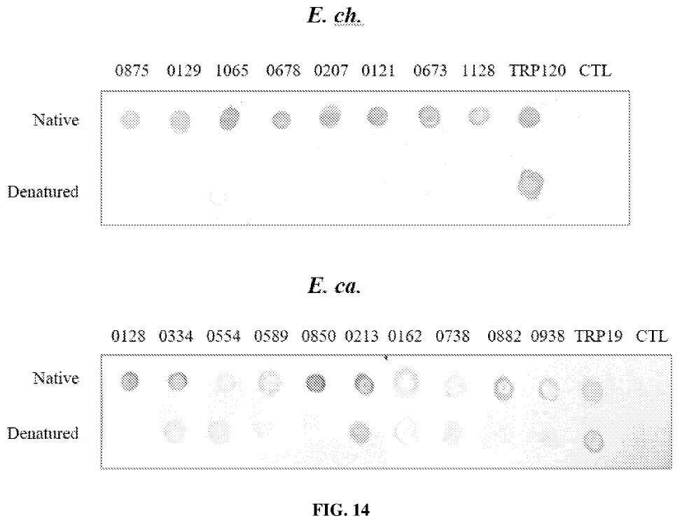

The conformational dependence of epitopes in the new Ehrlichia immunoreactive proteins was also examined by dot immunoblot (FIG. 14). Top new E. ch. and E. ca. immunoreactive proteins were selected and the immunoreactivity of native proteins was compared with that of denatured proteins using HME or CME serum. After denaturation, none of the new E. ch immunoreactive proteins reacted with the HME serum except that Ech_1065 reacted weakly. These results are consistent with our ELISA data in FIG. 10A and support the conclusion that these E. ch. immunoreactive proteins are defined by conformation-dependent antibody epitopes. After denaturation, one protein (Ecaj_0213) reacted strongly with the CME serum, but four proteins (Ecaj_0128, 0589, 0850 and 0882) did not react. Denatured Ecaj_0334 reacted with CME serum at a substantially lower level compared to native proteins, whereas the other four proteins reacted at a level similar to the native proteins. These results are consistent with our ELISA data in FIG. 11A, demonstrating our conclusion that the new E. ca. immunoreactive proteins contain major conformational epitopes, linear epitopes, or both.

Bioinformatic Analysis of New E. ch. And E. ca. Immunoreactive Proteins

A comprehensive bioinformatic analysis of the top 22 E. ch. and 18 E. ca. new immunoreactive proteins was performed using multiple online prediction tools (Table Z3 and Table Z4). Notably by TMHMM 2.0 server, 18 (82%) of E. ch. and 10 (56%) of E. ca. proteins were predicted to contain 1-6 transmembrane helixes. However, using SignalP 5.0 and SecretomeP 2.0, only three E. ch. (0678, 0044 and 1038) and one E. ca. protein (0818) were predicted to secreted by a standard secretory signal peptide or a non-classical (i.e., not signal peptide directed) mechanism. Since type I and type IV secretion systems (T1SS and T4SS) are present in E. ch. and E. ca., we examined these proteins as possible T1 and T4 substrates. Sequence analysis did not identify a consensus type IV secretory motif R—X(7)-R—X—R—X—R in any of the proteins (30). Only three E. ch. (0129, 0040 and 1038) and two E. ca proteins (0334 and 0728) were predicted to be type IV substrates by the S4TE 2.0 tool (31). In contrast, statistical analysis of the last 50 C-terminal residues of these proteins identified a putative type I secretion signal (LDAVTSIF-enriched (SEQ ID NO: 77); KHPMWC-poor (SEQ ID NO: 78)) described previously (32), suggesting that the majority of these proteins are type I secreted substrates. Ech_0875 protein showed the greatest difference between the residue occurrences of LDAVTSIF (SEQ ID NO: 77) (72%) and KHPMWC (SEQ ID NO: 78) (8%) in the last 50 C-terminal amino acids, whereas the predicted type IV substrate Ech_0129 showed the least difference (44% vs. 34%). Moreover, the PREFFECTOR server identified 11 (50%) E. ch. and eight (44%) E. ca. proteins as effectors (probability threshold=0.8) (33). This analysis supports the conclusion that many of these proteins are type I secreted effectors, although additional experimental validations are required (Table Z3 and Table Z4).

TABLE Z3
Predicted features of new E. ch. immunoreactive proteins

| | Amino | | | | | |
|---|---|---|---|---|---|---|
| Protein | acids/ | Transmembrane | | | | |
| (Ech_ tag | Mass | domains | Secretion | T4S | Effector | |
| no.) | (kD) | (TMHMM) | (SecretomeP) | (S4TE) | (PREFFECTOR) | Annotation |
| 0875 | 226/25 | + | − | − | − | putative |
| | | | | | | phosphatidylglycerophosphatase A |
| 0129 | 416/46 | + | − | + | − | HemY domain protein |
| | | | | | | 2-oxoglutarate |
| 1065 | 404/44 | − | − | − | − | dehydrogenase, E2 |
| | | | | | | component |
| 0678 | 230/25 | + | +^{a} | − | + | hypothetical protein |
| 0207 | 176/19 | + | − | − | + | hypothetical protein |
| 0121 | 368/40 | − | − | − | − | hypothetical protein |
| 0673 | 256/28 | + | − | − | + | hypothetical protein |
| 1128 | 196/22 | + | − | − | + | hypothetical protein |
| 0670 | 266/29 | + | − | − | − | hypothetical protein |
| 0706 | 108/12 | − | − | − | + | hypothetical protein |
| 0518 | 291/32 | + | − | − | − | hypothetical protein |
| 1055 | 175/19 | + | − | − | − | cytochrome C oxidase |
| | | | | | | assembly protein |
| 0640 | 295/32 | − | − | − | + | hypothetical protein |
| 0040 | 714/79 | + | − | + | + | type IV secretion system |
| | | | | | | component VirD4 |
| 0720 | 759/80 | + | − | − | − | hypothetical protein |
| | | | | | | sensor histidine |
| 0755 | 828/91 | + | − | − | − | kinase/response regulator |
| 0947 | 138/15 | + | − | − | − | hypothetical protein |
| 0044 | 237/26 | + | + | − | + | type IV secretion system |
| | | | | | | protein VirB8 |
| 0988 | 208/23 | + | − | − | + | hypothetical protein |
| 0635 | 357/39 | + | − | − | + | hypothetical protein |
| 0681 | 221/24 | + | − | − | − | hypothetical protein |
| 1038 | 1963/216 | + | +^{a} | + | + | EtpE |

^{a}Signal peptide predicted by SignalP

Among the top 22 E. ch. and 18 E. ca. new immunoreactive proteins, the majority (21/40; 53%) were hypothetical, consistent with our previous findings. In addition, eight E. ch. and 11 E. ca. proteins were annotated with putative function, and interestingly, most were enzymes involved in important biological processes, such as phosphatase, dehydrogenase, oxidase, kinase, isomerase, polymerase, deformylase, and peptidase. Notably, two type IV secretion system components (VirD4 and VirB8) and an entry-triggering protein of E. ch. (EtpE) were also identified as immunoreactive.

Although E. ca. and E. ch. orthologs for most of the new E. ch. and E. ca immunoreactive proteins were identified, only one ortholog pair (Ech_1065 and Ecaj_0857), annotated as 2-oxoglutarate dehydrogenase E2 component, was found to react strongly and consistently with HME or CME sera (Table Z1 and Table Z2). We also found that some other E. ca. and E. ch. orthologs were both immunoreactive, but these pairs did not react similarly with HME or CME sera. Moreover, some new E. ch./E. ca. antigens do not have corresponding orthologs and more antigens were found from E. ch. than E. ca. These findings suggest that the antibody epitopes in majority of new immunoreactive proteins are not conserved among corresponding ortholog pairs from E. ch. and E. ca. Most of the new E. ca. immunoreactive proteins exhibited conformational epitopes; however, major epitopes of all new E. ch. immunoreactive proteins appeared to conformation-dependent. This difference also highlights that these observations are fundamentally different from previously defined linear epitopes in major immunoreactive proteins of Ehrlichia (4). These results further demonstrated that E. ch. and E. ca. proteins have a divergence in antibody recognition and different conformational immunomes, a finding in contrast with previously defined linear epitope-containing proteins, and support the idea that these proteins can be used in vaccines.

The majority of the new immunoreactive proteins of E. ch. and E. ca. are predicted to be membrane proteins that contain at least one transmembrane domain. To the knowledge of the inventors, this feature has not been previously reported. The significance of transmembrane domains in immunoreactive proteins is unclear, although it further highlights the importance of ehrlichial proteins with transmembrane domains as targets of the host immune response. In addition, only a few of new immunoreactive proteins were predicted to be secreted, and only three E. ch. and two E. ca. proteins were predicted to be T4SS substrates by S4TE. However, the majority of new immunoreactive proteins were predicted to be type I secreted effectors. It is anticipated that these new immunoreactive proteins may be involved in many different interactions with the host cell during infection and can be targets that could be neutralized by antibody.

In this and recent studies, we have analyzed all proteins in top 350 according to ANTIGENpro and all hypothetical proteins present in E. ch. and E. ca. ORFeomes, and discovered numerous new immunoreactive proteins, including 18 E. ch. and 20 E. ca. immunodominant proteins. It is anticipated that this significant expansion of Ehrlichia immunoreactive proteins represents a relatively complete antigen repertoires of E. ch. and E. ca., and also highlights the likely importance of conformation-dependent antibody epitopes in immunity. These new immunoreactive proteins may rival TRPs for developing both sensitive diagnostics and as subunit vaccines for HME and CME. The data support these new Ehrlichia antigens for use in immunodiagnostics for early detection of antibodies against tick-specific ehrlichial proteins and/or for developing transmission-blocking vaccines.

### Example 4

### Materials and Methods

The following methods were used in the experiments in Example 3.

Gene synthesis. E. ch. (Arkansas strain) and E. ca. (Jake strain) gene sequences are available in the Integrated Microbial Genomes (IMG) (48) and GenBank. E. ch and E. ca. genes were codon-optimized and chemically synthesized by GenScript (Piscataway, N.J.).

HME and CME antisera. HME patient sera were kind gifts from the Centers for Disease Control and Prevention (Atlanta, Ga.), Vanderbilt University (Nashville, Tenn.), Washington University and St. Louis Children's Hospital (St. Louis, Mo.). CME sera were obtained from naturally infected dogs from the United States and Colombia. In order to avoid the possibility of polyreactive antibodies (IgM), which have been previously described, only convalescent sera and anti-IgG secondary antibodies were used to examine the immunoreactivity (49).

IVTT. In vitro expression of ehrlichial proteins was performed using the S30 T7 high-yield protein expression system (Promega, Madison, Wis.) according to the instructions from the manufacturer. The ehrlichial ORFs were cloned into pET-14b vector containing a 6×His-tag sequence, and crude plasmids were extracted by GenScript. Each plasmid was transformed into Stellar competent cells (Takara, Mountain View, Calif.) and purified from culture of a single colony using QIAprep spin miniprep kit (Qiagen, Germantown, Md.). The purified plasmid was mixed with the E. coli extract and a reaction premix and incubated at 37° C. for 3 h.

Dot immunoblot. The expression of ehrlichial proteins by IVTT was confirmed by dot immunoblot with horseradish peroxidase (HRP)-labeled His-tag mouse antibody (1:500; GenScript) as described previously (21). The immunoreactivity of native and denatured ehrlichial proteins was also examined by dot immunoblot with HME or CME antiserum, for which IVTT-expressed proteins were purified by MagneHis protein purification system (Promega) according to the instructions from the manufacturer. TMB 1-component substrate (Kirkegaard & Perry Laboratories, Gaithersburg, Md.) was used for all dot immunoblots.

ELISA immunoscreening. The immunoreactivity of Ehrlichia IVTT-expressed proteins with a 6×His-tag was determined by a His-tagged antigen-capture ELISA as described previously (21). HME or CME sera were diluted 1:200. Alkaline phosphatase-labeled rabbit anti-human IgG (H+L) secondary antibody (1:5,000; Abcam, Cambridge, Mass.) and BluePhos substrate (Kirkegaard & Perry) were used, and optical density was measured at 650 nm (OD₆₅₀). Dilution buffer containing 4 M urea was used to denature IVTT-expressed proteins and the diluted protein was incubated for 10 min at 99° C. ELISA OD values represent the mean OD reading from three wells (±standard deviation) after background subtraction. Since negative controls generally had raw readings of ˜0.1 OD, a sample OD of ≥0.2 was considered positive after subtracting negative control OD reading.

Peptide ELISA. To identify linear antibody epitopes in Ehrlichia immunoreactive proteins, ELISAs were performed using overlapping peptides (19-20 amino acids; 6 amino acids overlapped) commercially synthesized by GenScript (5). All peptides were supplied as a lyophilized powder and resuspended in molecular biology grade water (1 mg/ml).

Indirect fluorescent-antibody assay (IFA). The antibody titers of sera from HME patients and CME dogs were determined by IFA as previously described (21). Antigen slides were prepared from E. ch. (Arkansas)-infected THP-1 cells or E. ca. (Jake)-infected DH82 cells. A fluorescein isothiocyanate (FITC)-labeled rabbit anti-human or anti-dog IgG (H+L) secondary antibody (Kirkegaard & Perry Laboratories) was used. Slides were examined with a BX61 epifluorescence microscope (Olympus, Japan).

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,220,450
U.S. Pat. No. 4,373,932
U.S. Pat. No. 4,472,509
U.S. Pat. No. 4,897,268
U.S. Pat. No. 4,938,948
U.S. Pat. No. 4,938,948
U.S. Pat. No. 5,075,109
U.S. Pat. No. 5,440,013
U.S. Pat. No. 5,446,128
U.S. Pat. No. 5,470,723
U.S. Pat. No. 5,470,932
U.S. Pat. No. 5,543,504
U.S. Pat. No. 5,552,157
U.S. Pat. No. 5,565,213
U.S. Pat. No. 5,567,434
U.S. Pat. No. 5,618,914
U.S. Pat. No. 5,656,016
U.S. Pat. No. 5,670,155
U.S. Pat. No. 5,697,899
U.S. Pat. No. 5,710,245
U.S. Pat. No. 5,738,868
U.S. Pat. No. 5,741,516
U.S. Pat. No. 5,770,219
U.S. Pat. No. 5,779,708
U.S. Pat. No. 5,783,208
U.S. Pat. No. 5,795,587
U.S. Pat. No. 5,797,898
U.S. Pat. No. 5,840,833
U.S. Pat. No. 5,853,744
U.S. Pat. No. 5,859,184
U.S. Pat. No. 5,891,506
U.S. Pat. No. 5,929,237
U.S. Pat. No. 6,136,610
U.S. Pat. No. 6,210,708
U.S. Pat. No. 6,372,445
U.S. Pat. No. 6,617,142
U.S. Pat. No. 6,875,750
U.S. Pat. No. 6,951,765
U.S. Pat. No. 7,163,677
U.S. Pat. No. 7,282,194
U.S. Pat. No. 7,344,893
U.S. Pat. No. 7,371,582
U.S. Pat. No. 8,580,280
U.S. Pat. No. 8,980,288
U.S. Pat. No. 9,545,439
U.S. Pat. No. 10,117,921
U.S. Pat. No. 10,238,736
U.S. Patent Application 2005/0047972
U.S. Patent Application 2005/0047972
U.S. Patent Application 2005/0065463
U.S. Patent Application 2005/0250141
U.S. Patent Application 2007/0264664
U.S. Patent Application 2009/0005535
U.S. Patent Publication 2019/0008953
U.S. Patent Publication 2019/0038737
WO2005087803
EP2433646
Andrade et al., Scientific reports 9, 16258, 2019.
Atherton and Sheppard, 1989.
Aucouturier, et al., Expert Rev. Vaccines 1, 111-118, 2002.
Barbour et al., Infect Immun 76:3374-89, 2008.
Billeskov, et al., PLoS One 11, e0161217, 2016.
Budachetri et al., mBio 11, 2020.
Calabro et al., Vaccine 31: 3363-3369, 2013.
Carpino et al., Org. Proc. Res. Dev., 7(1) 28-37, 2003.
Chen et al., Clin.Diag.Lab.Immunol. 4, 731-735, 1997.
Chen et al., Nucleic Acids Res 47: D666-D677, 2019.
Chin et al., Applied biochemistry and biotechnology 174, 1897-1906, 2014.
Cruz-Fisher et al., Infect Immun 79, 246-257, 2011.
Delepelaire, Biochimica et biophysica acta 1694, 149-161, 2004.
Dhroso et al., Scientific reports 8, 17209, 2018.
Didierlaurent, et al., J. Immunol. 183: 6186-6197, 2009.
Didierlaurent, et al., J. Immunol. 193, 1920-1930, 2014.
Doyle et al., Infect Immun 74, 711-720, 2006.
Dumler et al., Clin. Infect. Dis., 45: S45-S51, 2007.
Dunphy et al., Microbes and infection/Institut Pasteur 15, 1005-1016, 2013.
Edwards and He, Methods Mol Biol. 907:281-92, 2012.
Felgner et al., Proc Natl Acad Sci USA, 106:13499-504.
Feng and Walker, Infect. Immun., 72:966-971, 2004.
Fishbein et al., AnnlnternMed 120:736-743, 1994.
Garçon and Van Mechelen, Expert Rev. Vaccines 10, 471-486, 2011.
Geysen et al., Proc. Natl. Acad. Sci. USA, 81(13):3998-4002, 1984.
Guan & Rosenecker, Gene Ther. 24, 133-143, 2017.
Harrus and Waner, Veterinary journal, 187, 292-296, 2011.
He et al., Hum Vaccin Immunother, 16:1554-64, 2020.
He et al., Human vaccines & immunotherapeutics, 1-11, 2020.
He et al., J Biomed Biotechnol 2010: 297505, 2010.
Hotopp et al., PLoS Genet 2: e21, 2006.
Jones et al., J Immunol 189:1440-7, 2012.
Kariko et al., Nucleic Acids Res. 39, e142, 2011.
Kariko, et al., Mol. Ther. 16, 1833-1840, 2008.
Kauffman, et al., J. Control. Release 240, 227-234, 2016.
Koczula et al., 2016
Künzi, et al., Vaccine 27, 3561-3567, 2009.
Kuriakose et al., Microbes and infection/Institut Pasteur 14, 1054-1063, 2012.
Kuriakose et al., PloS one 6, e24136, 2011.
Li and Winslow, InfectImmun 71:4229-4237, 2003.
Li et al., JImmunol 169:1419-1425, 2002.
Li et al., J. Immunol. 166, 1855-1862, 2001.
Lin et al., Front Microbiol 2:24, 2011.
Lina et al., Frontiers in cellular and infection microbiology 6, 58, 2016.
Liu et al., PloS one 14, e0210351, 2019.
Luo and McBride, 2003.
Luo and McBride, Infect Immun 80, 2297-2306, 2012.
Luo et al., Clinical and vaccine immunology: CVI 17, 87-97, 2010.
Luo et al., Clinical and vaccine immunology: CVI 16, 982-990, 2009.
Luo et al., Front Cell Infect Microbiol.; 7: 178, 2017.
Luo et al., Infect Immun 76, 1572-1580, 2008.
Luo et al., Infect Immun 79, 4382-4391, 2011.
Luo et al., mSphere 3, 2018.
Luo et al., NPJ Vaccines 5:85, 2020.
Magnan et al., Bioinformatics 26, 2936-2943, 2010.
McBride and Walker, Expert review of vaccines 9, 1071-1082, 2010.
McBride and Walker, Expert reviews in molecular medicine 13, e3, 2011.
McBride et al., American Association of Veterinary Laboratory Diagnosticians, Inc 8, 441-447, 1996.
McBride et al., Infect Immun 71, 2516-2524, 2003.
McBride et al., Infect Immun 75, 74-82, 2007.
McBride et al., Infect Immun 79, 3178-3187, 2011.
McBride et al., J Clin Microbiol 39, 315-322, 2001.
Mizuno et al., Chemistry. 23(58):14394-14409, Oct. 17 2017.
Morel, S. et al., Vaccine 29, 2461-2473, 2011.
Nandi et al., Infect Immun 75:5453-5459, 2007.
Nethery et al., Infect Immun 75, 4900-4908, 2007.
Noroy et al., PLoS computational biology 15, e1006847, 2019.
Ohashi et al., J Clin Microbiol 36, 2671-2680, 1998a.
Ohashi et al., Infect Immun 66, 132-139, 1998b.
Olano et al., EmerglnfectDis 9:1579-1586, 2003.
Paddock and Childs, Clinical microbiology reviews 16, 37-64, 2003.
Paparone et al., Ehrlichiosis with pancytopenia and ARDS. New Jersey Med 92:381-385, 1995.
Pardi et al., Nat Rev Drug Discov. 17(4): 261-279, 2018.
Paterson et al., Anal Chem. 86(19):9481-8, October 7; 2014.
Pierce Immunotechnology Catalog and Handbook, at A12-A13, 1991
Portnyagina et al., Int J Biol Macromol 107, 2484-2491, 2018.
Racine et al., JImmunol 186:1011-1021, 2011.
Remington: The Science and Practice of Pharmacy, 21^{st} Ed. Lippincott Williams and Wilkins, 2005.
Remington: The Science and Practice of Pharmacy; Pharmaceutical Press, 22nd Revised edition, 2012
Remington's Pharmaceutical Sciences, 18th Ed. Mack Printing Company, 1289-1329, 1990.
Saeed et al., Front Microbiol. 8: 495, 2017.
Safdari et al., Biotechnol Genet Eng Rev. 29:175-86, 2013.
Sahin et al., Nature Reviews Drug Discovery 13:759-780, 2014.
Seo et al., Infect. Immun. 76, 4823-4832, 2008.
Singu et al., Cell Microbiol. 8, 1475-1487, 2006.
Sotomay et al., AmJPath 158:757-769, 2001.
Stuen et al., Acta Vet Scand 57:40, 2015.
Sutten et al., Infect Immun 78:1314-25, 2010.
Swanson et al., Science Immunology: Vol. 5, Issue 47: p. 1-11, May 2020.
Tandrup Schmidt, et al., Pharmaceutics 8, 7, 2016.
The Science and Practice of Pharmacy, 21^{st} Ed. Lippincott Williams and Wilkins, 2005
Thess et al., Mol. Ther. 23, 1456-1464, 2015.
Van Regenmortel, Mapping Epitope Structure and Activity: From One-Dimensional Prediction to Four-Dimensional Description of Antigenic Specificity. Methods 9:465-72, 1996.
Vega et al., Vaccine 25:519-525, 2007.
Vergunst et al., Proceedings of the National Academy of Sciences of the United States of America 102, 832-837. 2005.
Vigil et al., Mol Cell Proteomics, 10: M110 006304, 2011.
Vigil et al., Future Microbiol, 5:241-51, 2010a.
Vigil et al., PLoS One 5: e11447, 2010b.
Wakeel et al., Frontiers in cellular and infection microbiology 1, 22, 2011.
Wakeel et al., InfectImmun 77, 1734-1745, 2009.
Walker and Dumler, Archives of Pathology & Laboratory Medicine 121:785-791, 1997.
Walker et al., Trans Am Clin Climatol Assoc 115:375-382; discussion 382-374, 2004.
Wang et al., 2017
Winslow et al., Ann. NYAcad. Sci., 990:435-443, 2003.
Winslow et al., Infect. Immun., 68:2187-2195, 2000.
Winslow et al., InfectImmun 66:3892-3899, 1998.
Yager et al., Infect. Immun., 73:8009-8016, 2005.
Yu et al., Scientific reports 9, 16094, 2019.
Zemella et al., Chembiochem.;16(17):2420-2431, 2015.

What is claimed is:

1. A pharmaceutical composition comprising a nucleic acid comprising an open reading frame encoding a polypeptide of Table 1, Table 2, Table 3, Table 4, or Table 5 formulated in a lipid nanoparticle or a viral vector.

2. The pharmaceutical composition of claim 2, wherein the nucleic acid is a ribonucleic acid (RNA).

3. The pharmaceutical composition of claim 2, wherein the nucleic acid is an mRNA further comprising a 5′ untranslated region (UTR) and a 3′ UTR.

4. The pharmaceutical composition of claim 3, wherein the mRNA comprises at least one analogue of a naturally occurring nucleotide or wherein the mRNA is chemically modified.

5. The pharmaceutical composition of claim 4, wherein the analogue is selected from the group consisting of phosphorothioates, phosphoramidates, peptide nucleotides, methylphosphonates, 7-deazaguanosine, 5-methylcytosine, and inosine.

6. The pharmaceutical composition of claim 4 wherein the mRNA comprises pseudouridine, a 5′ cap analog, or a poly(A) tail.

7. The pharmaceutical composition of claim 6, wherein the 5′ cap analog is 7mG(5′)ppp(5′)NlmpNp.

8. The pharmaceutical composition of claim 6, wherein the chemical modification is a 1-methylpseudouridine modification or a 1-ethylpseudouridine modification.

9. The pharmaceutical composition of any one of claims 2-8, wherein the mRNA comprises a 5′ untranslated region (UTR) and a 3′ UTR.

10. The pharmaceutical composition of any one of claims 3-9, wherein the mRNA is comprised in liposomes, lipid nanoparticles, or a viral vector.

11. The pharmaceutical composition of claim 10, wherein the liposomes or lipid nanoparticles comprise an ionizable cationic lipid, a neutral lipid (e.g., DSPC), sterol (e.g., cholesterol), and/or a PEG-modified lipid (e.g., PEG-DMG or PEG-DMA).

12. The pharmaceutical composition of any one of claims 2-11, wherein the RNA encodes the polypeptide of Table 1, Table 2, Table 3, Table 4, or Table 5 for secretion.

13. The pharmaceutical composition of any one of claims 2-11, wherein the RNA encodes the polypeptide of Table 1, Table 2, Table 3, Table 4, or Table 5 as an intracellular protein.

14. The pharmaceutical composition of any one of claims 2-11, wherein the polypeptide of Table 1, Table 2, Table 3, Table 4, or Table 5 is comprised in a fusion protein.

15. The pharmaceutical composition of claim 12, wherein the fusion protein comprises a transmembrane region.

16. The pharmaceutical composition of claim 1, wherein nucleic acid is a DNA.

17. The pharmaceutical composition of claim 16, wherein the DNA is comprised in a viral vector.

18. The pharmaceutical composition of claim 16, wherein the viral vector is an adenovirus, or an adeno-associated virus (AAV).

19. A pharmaceutical composition comprising a polypeptide of Table 1, Table 2, Table 3, Table 4, or Table 5 and an excipient.

20. The pharmaceutical composition of claim 19, wherein the composition further comprises an adjuvant.

21. The pharmaceutical composition of claim 20, wherein the adjuvant comprises a triterpenoid saponin, a sterol, and/or an immunostimulatory oligonucleotide.

22. The pharmaceutical composition of claim 21, wherein the triterpenoid saponin is Quil A.

23. The pharmaceutical composition of any one of claims 21-22, wherein the immunostimulatory oligonucleotide is a CpG-containing ODN.

24. The pharmaceutical composition of any one of claims 21-23, wherein the CpG-containing ODN is 5′ JU*C-G*T*C*G*A*C*G*A*T*C*G*G*C*G*G*C*C*G*C*C*G*T 3′ (SEQ ID NO: 75), wherein “*” refers to a phosphorothioate bond, “-” refers to a phosphodiester bond, and “JU” refers to 5′-Iodo-2′-deoxyuridine.

25. The pharmaceutical composition of any one of claims 19-24, wherein the composition comprises an E. canis bacterin or an E. chaffeensis bacterin.

26. The pharmaceutical composition of claim 25, wherein the E. canis bacterin or the E. chaffeensis bacterin is a heat-inactivated or chemically-inactivated bacterin.

27. The pharmaceutical composition of claim 26, wherein the chemically-inactivated bacterin was inactivated with formaldehyde, formalin, bi-ethylene amine, radiation, ultraviolet light, beta-propiolactone treatment, or formaldehyde.

28. A method of detecting antibodies that specifically bind an Ehrlichia organism in a test sample, comprising: (a) contacting an isolated polypeptide comprising or consisting of a sequence of Table 1, Table 2, Table 3, Table 4, or Table 5 or a polypeptide having at least 95% sequence identity thereto, with the test sample, under conditions that allow peptide-antibody complexes to form; (b) detecting the peptide-antibody complexes; wherein the detection of the peptide-antibody complexes is an indication that antibodies specific for an Ehrlichia organism are present in the test sample, and wherein the absence of the peptide-antibody complexes is an indication that antibodies specific an Ehrlichia organism are not present in the test sample.

29. The method of claim 28, wherein the polypeptide is selected from the group consisting of a polypeptide of Table 2.

30. The method of claim 28, wherein the polypeptide is selected from the group consisting of a polypeptide of Table 3.

31. The method of claim 28, wherein the polypeptide is selected from the group consisting of a polypeptide of Table 4 or Table 5.

32. The method of any one of claims 28-31, wherein the Ehrlichia organism is an Ehrlichia chaffeensis organism.

33. The method of any one of claims 28-31, wherein the Ehrlichia organism is an Ehrlichia canis organism.

34. The method of any one of claims 28-33, wherein the step of detecting comprises performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a mass spectrometry assay, or a particulate-based assay.

35. The method of claim 34, wherein the step of detecting comprises a lateral flow assay or an enzyme-linked immunoassay, wherein the enzyme-linked immunoassay is an ELISA.

36. The method of any one of claim 28 or 32-35, wherein the isolated polypeptide is Ech_0991, Ecaj_0126, Ecaj_0717, Ecaj_0636, Ecaj_0920, Ecaj_0259, or Ecaj_0348.

37. The method of any one of claim 28 or 32-35, wherein the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055.

38. The method of any one of claim 28 or 32-35, wherein the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

39. A method of identifying an Ehrlichia infection in a mammalian subject comprising: (a) contacting a biological sample from the subject with an isolated polypeptide comprising or consisting of a sequence of Table 1, Table 2, Table 3, Table 4, or Table 5 under conditions that allow peptide-antibody complexes to form; and (b) detecting the peptide-antibody complexes; wherein the detection of the peptide-antibody complexes is an indication that the subject has an Ehrlichia infection.

40. The method of claim 39, wherein the polypeptide is selected from the group consisting of Table 2.

41. The method of claim 39, wherein the polypeptide is selected from the group consisting of Table 3.

42. The method of any one of claims 39-41, wherein the step of detecting comprises performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a dipstick test, or a particulate-based assay.

43. The method of claim 39, wherein the subject is a human.

44. The method of claim 39, wherein the subject is a dog.

45. The method of any one of claim 39 or 42-44, wherein the isolated polypeptide is Ech_0991, Ecaj_0126, Ecaj_0717, Ecaj_0636, Ecaj_0920, Ecaj_0259, or Ecaj_0348.

46. The method of any one of claim 39 or 42-44, wherein the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055.

47. The method of any one of claim 39 or 42-44, wherein the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

48. An isolated polypeptide comprising or consisting of a sequence of Table 1, Table 2, Table 3, Table 4, or Table 5, wherein the isolated polypeptide is immobilized on a surface of a support substrate.

49. The polypeptide of claim 48, wherein the polypeptide is selected from the group consisting of Table 3.

50. The polypeptide of claim 49, wherein the polypeptide is selected from Table 4 or Table 5.

51. The polypeptide of any one of claims 48-50, wherein the support substrate comprises latex, polystyrene, nylon, nitrocellulose, cellulose, silica, agarose, or magnetic resin.

52. The polypeptide of any one of claims 48-51, wherein the support substrate is a reaction chamber, a well, a membrane, a filter, a paper, an emulsion, a bead, a microbead, a dipstick, a card, a glass slide, a lateral flow apparatus, a microchip, a comb, a silica particle, a magnetic particle, a nanoparticle, or a self-assembling monolayer.

53. The polypeptide of any one of claims 48-52, wherein the polypeptide is comprised in a kit.

54. The polypeptide of any one of claims 48-52, wherein the polypeptide is produced via peptide synthesis or in vitro transcription and translation (IVTT).

55. The polypeptide of any one of claims 48-52, wherein the polypeptide is recombinantly produced.

56. The polypeptide of any one of claim 48 or 51-55, wherein the isolated polypeptide is Ech_0991, Ecaj_0126, Ecaj_0717, Ecaj_0636, Ecaj_0920, Ecaj_0259, or Ecaj_0348.

57. The polypeptide of any one of claim 48 or 51-55, wherein the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055.

58. The polypeptide of any one of claim 48 or 51-55, wherein the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

59. An isolated polypeptide comprising or consisting of a sequence of Table 1, Table 2, Table 3, Table 4, or Table 5, wherein the isolated polypeptide is covalently attached to or bound to a detectable label.

60. The polypeptide of claim 59, wherein the polypeptide is selected from the group consisting of Table 3.

61. The polypeptide of claim 59, wherein the polypeptide is selected from Table 4 or Table 5.

62. The polypeptide of any one of claims 59-61, wherein the detectable label a fluorescent label, a radioactive label, an enzyme label, or a luminescent nanoparticle.

63. The polypeptide of claim 62, wherein the luminescent nanoparticle is a luminescent rare earth nanoparticle, a luminous nanoparticle, or a strontium aluminate nanoparticle.

64. The polypeptide of any one of claims 59-63, wherein the polypeptide is comprised in a kit.

65. The polypeptide of any one of claims 59-64, wherein the polypeptide is produced via peptide synthesis or in vitro transcription and translation (IVTT).

66. The polypeptide of any one of claims 59-64, wherein the polypeptide is recombinantly produced.

67. The polypeptide of any one of claim 59 or 62-66, wherein the isolated polypeptide comprises or consists of Ech_0991, Ecaj_0126, Ecaj_0717, Ecaj_0636, Ecaj_0920, Ecaj_0259, or Ecaj_0348.

68. The polypeptide of any one of claim 59 or 62-66, wherein the isolated polypeptide is Ech_0875, Ech_0129, Ech_1065, Ech_0678, Ech_0207, Ech_0121, Ech_0673, Ech_1128, Ech_0670, Ech_0706, Ech_0518, or Ech_1055.

69. The polypeptide of any one of claim 59 or 62-66, wherein the isolated polypeptide is Ecaj_0151, Ecaj_0128, Ecaj_0213, Ecaj_0162, Ecaj_0554, Ecaj_0857, Ecaj_0334, Ecaj_0104, or Ecaj_0737.

70. A kit comprising: (a) the isolated polypeptide of any one of claims 59-69, (b) an anti-dog or anti-human secondary antibody linked to a reporter molecule; and, (c) an appropriate reagent for detection of the reporter molecule.

71. The kit of claim 70, wherein the polypeptide is immobilized on a membrane or a microtiter plate.

72. The kit of any one of claims 70-71, wherein the reporter molecule is selected from the group consisting of luciferase, horseradish peroxidase, a luminous nanoparticle, P-galactosidase, and a fluorescent label.

73. The kit of claim 72, wherein the luminous nanoparticle is a strontium aluminate nanoparticle.

74. The kit of claim of any one of claims 70-73, wherein the kit further comprises a dilution buffer for dog or human serum.

75. The kit of claim of any one of claims 70-74, wherein the kit comprises a lateral flow immunoassay or a lateral flow immunochromatographic assay.

76. The kit of claim of any one of claims 70-75, wherein the kit comprises an enzyme-linked immunosorbent assay (ELISA).

77. A method of inducing an immune response in a mammalian subject comprising administering to the subject an effective amount of a pharmaceutical preparation comprising an isolated polypeptide comprising or consisting of a polypeptide sequence of Table 1, Table 2, Table 3, Table 4, or Table 5, or a nucleic acid encoding a polypeptide sequence of Table 1, Table 2, Table 3, Table 4, or Table 5.

78. The method of claim 77, wherein the polypeptide comprises or consists of a polypeptide of Table 3, Table 4, or Table 5.

79. The method of claim 77, wherein the nucleic acid is an mRNA.

80. The method of claim 79, wherein the mRNA comprises at least one analogue of a naturally occurring nucleotide or wherein the mRNA is chemically modified.

81. The method of claim 80, wherein the analogue is selected from the group consisting of phosphorothioates, phosphoramidates, peptide nucleotides, methylphosphonates, 7-deazaguanosine, 5-methylcytosine, and inosine.

82. The method of claim 80 wherein the mRNA comprises pseudouridine, a 5′ cap analog, or a poly(A) tail.

83. The method of claim 82, wherein the chemical modification is a 1-methylpseudouridine modification or a 1-ethylpseudouridine modification.

84. The method of claim 82, wherein the mRNA comprises a 5′ untranslated region (UTR) and a 3′ UTR.

85. The method of any one of claims 79-84, wherein the mRNA is comprised in liposomes, lipid nanoparticles.

86. The method of claim 85, wherein the liposomes or lipid nanoparticles comprise an ionizable cationic lipid, a neutral lipid (e.g., DSPC), sterol (e.g., cholesterol), and/or a PEG-modified lipid (e.g., PEG-DMG or PEG-DMA).

87. The method of any one of claims 77-78, wherein the nucleic acid is a DNA.

88. The method of claim 87, wherein the DNA is comprised in a viral vector.

89. The method of claim 88, wherein the viral vector is an adenovirus, or an adeno-associated virus (AAV).

90. The method of claim 77, wherein the method comprises administering the pharmaceutical composition of any one of claims 1-27.

91. The method of any one of claims 77-90, wherein the subject is a human.

92. The method of any one of claims 77-90, wherein the subject is a dog.

93. The method of any one of claims 77-91, wherein the pharmaceutical preparation is administered subcutaneously, intramuscularly, nasally, via inhalation or aerosol delivery, or intradermally.

94. The method of any one of claim 77 or 91-93, wherein the isolated polypeptide comprises or consists of Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.

95. The method of any one of claims 77-94, wherein the method further comprises administering an Ehrlichia bacterin to the mammalian subject.

96. The method of any one of claims 77-95, wherein the method further comprises administering an adjuvant to the subject.

97. The method of any one of claims 77-96, wherein the polypeptide is comprised in a multimer or fusion protein.

98. A method of treating an Ehrlichia chaffeensis infection in a subject comprising:
(a) contacting a biological sample from the subject with an isolated polypeptide comprising or consisting of a sequence of Table 1, Table 2, Table 3, Table 4, or Table 5 under conditions that allow peptide-antibody complexes to form;
(b) detecting the peptide-antibody complexes; wherein the detection of the peptide-antibody complexes is an indication that the subject has an Ehrlichia chaffeensis infection; and
(c) administering a therapeutic compound to treat Ehrlichia infection in the subject.

99. The method of claim 98, wherein the polypeptide is selected from the group consisting of Table 2.

100. The method of claim 98, wherein the polypeptide is selected from the group consisting of Table 3.

101. The method of any one of claims 98-100, wherein the step of detecting comprises performing an enzyme-linked immunoassay, a radioimmunoassay, an immunoprecipitation, a fluorescence immunoassay, a chemiluminescent assay, an immunoblot assay, a lateral flow assay, a flow cytometry assay, a multiplex immunoassay, a dipstick test, or a particulate-based assay.

102. The method of claim 98, wherein the subject is a dog.

103. The method of claim 98, wherein the subject is a human.

104. The method of any one of claims 98-103, wherein the therapeutic compound is an antibiotic.

105. The method of claim 104, wherein the antibiotic is doxycycline.

106. The method of any one of claim 98 or 100-105, wherein the isolated polypeptide is Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.

107. An antibody that selectively binds a polypeptide of Table 1, preferably Table 3, Table 4, or Table 5.

108. The antibody of claim 107, wherein the polypeptide is a polypeptide of Table 3, Table 4, or Table 5.

109. The antibody of any one of claims 107-108, wherein the antibody is a polyclonal antibody.

110. The antibody of any one of claims 107-108, wherein the antibody is a monoclonal antibody.

111. The antibody of any one of claims 107-110, wherein the antibody is a mammalian antibody.

112. The antibody of any one of claims 107-110, wherein the antibody is a humanized antibody.

113. The antibody of any one of claims 107-112, wherein the antibody selectively binds Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.

114. The antibody of any one of claims 107-113, wherein the antibody is present in a multimer.

115. A method of detecting an ehrlichiosis infection in a mammalian subject, comprising:
(a) obtaining a biological sample from the mammalian subject, wherein the biological sample is preferably serum or blood; and
(b) performing a polymerase chain reaction (PCR) amplification that can selectively expand a nucleic acid encoding a polypeptide of Table 1;
wherein expansion of the nucleic acid indicates that the mammalian subject has ehrlichiosis.

116. The method of claim 115, wherein the polypeptide is a polypeptide of Table 3, Table 4, or Table 5.

117. The method of claim 116, wherein the polypeptide is Ech_0991, Ecaj_0126, Ecaj_0717, Ecaj_0636, Ecaj_0920, Ecaj_0259, Ecaj_0348, Ech_0875, Ech_0129, Ech_1065, Ech_0678, Ech_0207, Ech_0121, Ech_0673, Ech_1128, Ech_0670, Ech_0706, Ech_0518, Ech_1055, Ecaj_0151, Ecaj_0128, Ecaj_0213, Ecaj_0162, Ecaj_0554, Ecaj_0857, Ecaj_0334, Ecaj_0104, or Ecaj_0737.


SEQUENCE LISTING (78 sequences)

SEQ ID NO: 1 - protein, 226 aa, Ehrlichia chaffeensis
MKIIGKILPTRLISTFLGTGYLPAWQNHWAAVLSLILGYVLFYLIYGMSYVSYGILVTGAVVASLFLKISLGLIVISIISIFVFQSNNSADDRSDIIVVQIALGQLLVVALSMPAILAIYNYLAIFYSKICQNIFMCPSWFNDFMHFLMFFLIPFVFFNILDIIKPWPISSLQILYNNCFSIVFEGLVLVIYTLIVMYLVAFICFDLTIRDAVALNSYIFTLLKFR

SEQ ID NO: 2 - protein, 416 aa, Ehrlichia chaffeensis
MITSVLIFVIVALTIGLWVIDCDGIIRIDWLGYDIEVNILFTLFVIAVVFLLLILLVRFIFCFSRCVYRYKRDLQNKKMVLLEQGYMYLNCGDVERVEKIIVKIGNFDHPSLFLLKGRVYFDTGKYILAEKYFTQFVKVVPVIDASLGIHLLNVIMQIEDQIQQLSLLRKMLEIFFKQSWSAIFKLTIYRISRDWGNAIEEMKKIIKLKINLPLPYNTQEMLNVFYYALAKQCYDIQKYDDGLRVLDNIKNCSQQCSTAVTLLKAKFYIDTDKKRKAVNILEHEYRINPHPDIANFYLDIMQHSSHAIHKLYSFNTGYYFSIYLIAQDAINSGEYDTAMKYLNHSFKTKTYISLYFLVLKLKVLSQNYNELLYWTDKIAKDAIADKYWSCTKCKYTPTCWHYECDGCKSFNTIIWV

SEQ ID NO: 3 - protein, 404 aa, Ehrlichia chaffeensis
MSEIQVKAENLGGESILEAPIRVSVNVGDSVKQGDMLFIIETDKTSLEIVSPEDGIINEIFVVDEEIIQRGQVLCTINTVKSNAVKPSEGNTAHSTTVTVADDMQQFIQKKDAPSAMKIMEENVIDKSQVSGSGIGGRITKSDVLNYMKLASEEDNTKANSISSLSVVSEEKREERVKMSKIRQVIAARLKESQNTAAILTTFNEVDMKNVMDLRAKYRETFEKKYGIKLGFMSFFIKAVVLALKELPIINAEISGNEIVYKHYYDMGIAVGTDKGLVVPVIRDADKMSFADLESTLASLGKKAREGKLEVADMAGATFTITNGGVYGSLLSTPIINPPQSGILGMHSIQKRPVAIDDKTIEIRPMMYIALSYDHRIVDGQGAVTFLVRIKQYIEDPSRMFLEV

SEQ ID NO: 4 - protein, 230 aa, Ehrlichia chaffeensis
MKNYICVIVYFAVVTFNFDALANHFYLKGGYNLVGNYSDSFKSEYMGYKRFNIDINTAAGYQLSNNFFYEVKVRYANIKPAIQKLEYFEKIDLIDILQRIIDKKVYISKIEDIFKINSVTTLINCGYDYVINNKLIVYLSYGVGIAGLLNYQGFRSNVATHYGMSIQSEVGLCYLYSKKMNLCIGYDYLKNYWKYDTNKIYDEDGNTVVYHFQDFQLNSHTVFVDFKVIL

SEQ ID NO: 5 - protein, 176 aa, Ehrlichia chaffeensis
MLHITQNSDNIVHSFYIAIQKYFKFTNLKKHCNTFRFTNFYFNHTIKCRAANYIIYMQSFIIYKHPKKINTSDGIVFVQDEFSFMAFIFSILFTFYNKLWLLSFISIVILSGIYIMYNTLNLINLPIYLSVNLLYSLYIASSYPDWYQAKLKKMGYKIHDVIFAENLISAKLKFKR

SEQ ID NO: 6 - protein, 368 aa, Ehrlichia chaffeensis
MQHTAVPGMVAPTSVISAKHVVIKGLVYKHVKHYSIEEYKSQIKEFRESITCFARMHMSYMYHMLHNTFVVRNGRIMLKSEIEQCLSKITSNIRLCAFVIKIGIVDHVMSRLCRFYGSDSIKYCASHYHDPRVIDSILIGLYGASYSDFSRMSYQVRSNIVYCVGKHGIAGVFKLHNSGFYSELLGMCYDFVHARGKGVKLQELCDFMKLSCSIQLGQMYHMMVKVKCSIGDEQSDIRKLVSQECSVGYLVYRSLLFGRYAYHVRKAFRHLYAPSDKNPVRTVSGLNIPHSLIRLNHRGIFTKIEHCINAEKMSFNVFVVDIVRHIDKLLLHPREEVYIREDISTYCAIVSSRYSTMGPDIDSSYHIL

SEQ ID NO: 7 - protein, 256 aa, Ehrlichia chaffeensis
MIKIKASRYTKIHLIAIAIILSLWNVFFIIKLISSIPQHIKNLIFPYTAILSISSLLLYGINIIYNLNIMLKYHQGKIELQKKSLFSYLGVIGEATSLLALNIISTIAILILPMNKPMIIAASIFNILSSLFVIEYASIFLNTDIKNHKQLKNTNKSTKYTTWSIINWSAALIISIANVSFTFTGYFIKHNANDIFKIILFTVYISIITSLIFMRNMRENDPSILLNAQVEPTRFNTDEEREHLLKHEEGISHYSR

SEQ ID NO: 8 - protein, 196 aa, Ehrlichia chaffeensis
MIYKEKLTRVGEYILAYLSFILSTYIFLVLVNIIRYNSLAICVISLLRTNIFNVSTKKLIKDKCRDTKFSNMNCYLYGKPLNLQIFYGIFSFIRNFQNNTLIIPNDSKCGFYTTLWDNPALHYTYTLTGSEYRNFFDILYENIICQCKLLINYNRSVLNQHNKNTLVIIPIPNAREFSNEIRVRNISINKESSYEC

SEQ ID NO: 9 - protein, 266 aa, Ehrlichia chaffeensis
MFLFKKATYKTKYYIALCANTIYFGVFLFAIFIQLIRFPSKLSNKDLLLINVLMVLSARIMLCLLSSYELTCTHYTNDNDSLLYYKKAAYAAEVISTIIAIIIQVIAVSQVAAGNLEIVSSKKTTIHTKGAVDFACILIKLLIASPLLVYFNYRRMKDDKCAAYKKDAEILFYLSITSLVISFIAFIGKIINVLEQTQSFALFNTENHSNNGPTNFPLGPIIRISCIAASIIILTVILSIESSISSKMSDTTIEYQGLSNANELSG

SEQ ID NO: 10 - protein, 108 aa, Ehrlichia chaffeensis
MLFRSGPKTIGSAVEKLILNKCDKNHISKIEVLLFFNWNNIVGEEISQVAKPKKLSFLNAMNTGVLYLVVNNGGVAINIQYAIPIIIEKISVFFGFKVVNIIKIRQQL

SEQ ID NO: 11 - protein, 291 aa, Ehrlichia chaffeensis
MSSFKNLIIRTTGFVIGSLLLIFSINMLMRRCSKRSNNVSIAVILLFSIGITVFFQIYGRNEHSLTFVNIKMYVMLLASSLIMFCIANLVLRKESECYSKFREQNKKIYFLKIDIANLEIQLNIMKREIEWFFDNLCSDTKICVDIKQEYLLPANKKFYDIGFLCKTAKFVTFSIALVVFLCESFYSVLSQKFDLVGLFMYYVLISLFLFTFIIDRLALYSEKNYKSISQNSKSIIDTLNTELNVKKRMYTVLLQEVSKYNILTEKELVNENNIRTLEKYKNKIFDILDRV

SEQ ID NO: 12 - protein, 175 aa, Ehrlichia chaffeensis
MTPRIKNTIYILITIILSMVCLVYASVPLYSIFCKVTGYGGTVRKSNILSSSKTGNTTIKVRFNADINKQLPWKFYPELPYVFVKPGEQKLIFYRAENLSDKDISGMAVYNVTPHKAGKYFNKVACFCFSKQTLFPYQKTVMPVSFFIDPAIETDPETADVKLITLSYVFFKYKE

SEQ ID NO: 13 - protein, 295 aa, Ehrlichia chaffeensis
MMPLLHTVREKEGSQQIVKLYRKNSLIFLRKVVMGIRKIATKSWYNIPKEQQVSMDIFRKPILSMTATGVMQYSQGYDIVYKEISFKNKLNVEVFYRNCLLDIKWFSLAERRYHTRFIREKNRPVLCSYDGQIYSTNSKVAYVITLEGNLITHDHIVCDRTQGQTYFHSTLAAGMPVICAGLLSVSNGIIHYISNESGHYKPGIGNLYNAVKLLDKVISLDCIICVIGFAVNEETKKLRTVYQARVKGSFLVDMESVGKDGLTIPERYFSIIRKDNDNYKKRLIQAANSHNVQAR

SEQ ID NO: 14 - protein, 714 aa, Ehrlichia chaffeensis
MDSVSANHIRNILFLVLGAFFGLEFCFYLSGVLFILMVWGPDYLDFNAINPSFSDFPDRIWPTVFNYVEHWWHNPSSYDAILLVKLISSLCIPIGILSIVLWNLRNILFDWRPFKKKESLHGDSRWATEKDIRKIGLRSRKGILLGKDKRGYLIADGFQHALLFAPTGSGKGVGFVIPNLLFWEDSVIVHDIKLENYDLTSGWRKKRGQEVFVWNPAQPDGVSHCYNPLDWISSKPGQMVDDVQKIANLIMPEQDFWYNEARSLFVGVVLYLLAVPEKVKSFGEVVRTMRSDDVVYNLAVVLDTIGKKIHPVAYMNIAAFLQKADKERSGVVSTMNSSLELWANPLIDTATASSDFNIQEFKRKKVTVYVGLTPDNLTRLRPLMQVFYQQATEFLCRTLPSDDEPYGVLFLMDEFPTLGKMEQFQTGIAYFRGYRVRLFLIIQDTEQLKGIYEEAGMNSFLSNSTYRITFAANNIETANLISQLIGNKTVNQESLNRPKFLDLNPASRSLHISETQRALLLPQEVIMLPRDEQILLIESTYPIKSKKIKYYEDKNFTKKLLKSTFVPTQEPYDPNNVKITKKENEDPIPSIESDIPENKPNNTENNLTEEGAIYNSTETNYEDDEDEDDDFNFDDLDEYIDEDEEYSEQPEEDYDYTDEEDEDEEEDEEDNDYNNSNEDDDYENNNISNSHHENDFDNHNEKPVKDQDIKNDKQ

SEQ ID NO: 15 - protein, 729 aa, Ehrlichia chaffeensis
MYITRKVSINQFIALLSCCLICSIFLLKSVYKRLLTRKDENTPLPVEPEPIILNRYVPEYSTTILPEHYAANYSIHRNLCSISQNLKLLISQPYGISEMESYPCRNHYLNVVHGSRDVLLFPKFRGYFDYILDKLICAGMFVEHNPVDIRKVKDLLGEENFTFVILAAQLIIRNEALYATYSESAYDKIAMLYNLSIEEVPYQERLNSANKIIRLLGNKVYSFSSQDELCKFLSNVFREYMHDCVSEIGNENDFYRMVNNDQKFKLLQEIFCMSYFKYYCIYGHRSHAVVTQEIDLASNIIGQYDEPKAVDDEPIIERICNMRKSGFEVELIDNCCSVLNVQEIISQIFEQTVIENNRRVSSFETDKVTRLLGTIEDVIEKVKEVTHYDAVDVVSSGRDYYRLMINMAYSGPPYNRMFISKELSMIMNQEECLFSVFSSFYDYARIVDKVTWSLADFEECEAEAANRFKDAEYYILYTRLKAKLDVGLFILHTRPDLPMTQDQILKVWYNDDASQFGLDKHILKDVLRNIVCCARVVCCLPYLLHSYPENIVDYIRMLARLDIDIYCTANMIISYDSYDICVFKDQDEFQQMINSFLISYNEYYKDLDTGLDNSRVTFGSSFDIVLVSLMYTSLYNSSLVEQHVIDAACNRMKSFNFCYLERNYRSLMTIIAETVNCRHNSIYWPLCEMDLSNSIDMSQESNDESIGEKWYQHTILPVLGNSAMTRGSH

SEQ ID NO: 16 - protein, 828 aa, Ehrlichia chaffeensis
MKKRHTDNTQNNKVDFVIRRHGLSVCKLAIFLIAPLLCISSLYLFNIYDSYINIIINCIFTSISILAISIQLRQYLYTIASIEYQNMIFANSLNHNTEFCLIIKNNGKIIYADARFYERFIKNQERKLDLFDILKLGNISEQEIEAFENALKNKLSINTYFCLNTKNKISNFTLILDPVLENPEIEINYTKTVNLFFAPLARPQEYFVLKAVKITKEQVYERLIQKHCVGAYLLNHRGVILSVNDSFLKMFELNSVENGTTFANFLTKSDNKEKVIDNNTVTLSTTTGITFKAHISQAAFYDKNNHSYIYGLLTPVNSDILNYHLHPCFTEAPIAILQCNTDGKILKSNNTLKRLVKHSKEYIFEYILPSYNKKIKKYFQNNIVKNMSLEAQLYNNSYVKIHFNKFIHQNNMFIICYITDSEDRKNLEIQLEQSQKMQAIGQLAGGIAHDFNNILTAIIGFCDLLLIRHTATDPSFSDIMQIKQNANRATNLIKQLLAFSRKQTLQPKILDVNNIIADLLHMIKRLINESIELKIQYEQNINLVKADLCQLEQVIINLVVNARAAMETGGQLSIKTYNTKTDVLKVLLKDMFSPDKEQIEDGEYVAIEVSDTGHGMEDKIMKKIFDPFFSTKETTAGIGLGLSTVYGIVKQTDGYIYVKSIVNSGTTFIILIPTVHLSNNNDIEIPYNKYSNETSTTVPEVNTSTTILLIEDEDPVRIFTTKALTKKGFKVIDINCGDQALDIINKYPIDIVISDVVMPKISGPEIVEQILKVKPNIQIIFISGYAEEVFNKYSNIDVSKINFLSKPFTLKQLTEKVLEISDNSFLQN

SEQ ID NO: 17 - protein, 138 aa, Ehrlichia chaffeensis
MIEIYSLLFVDSFVAALILPLNKILIFKIMAYFGGYSYPLMLLVSTLGAVIGGVINWVLGRMIIFARVEYHKVQDDYGKLGVYIKLALMLLTLLCSWIPVWGGIVNVLSGYFRVEMLKLVVLLFLSYLGYLTYCIITL

SEQ ID NO: 18 - protein, 237 aa, Ehrlichia chaffeensis
MIRMFNKFRKKNNKNDNNKLSNANLETTYSWHVSRYNSVVIQRNMLLFFTMLALSAVGISVFVIFNISKNRTIEPFVVEIEKKSGITTLVNPVSVKQYSADEVLNNHFIIEYVRSRELFDPNNFQYNYYTKVRLFSNQTTYSEFRNWIRLSNPASPLNLYANVTSGYLKIRSLQHLRPGSVQIRFSLEFNHPSGIIKKDRIATLSFQYVTLEMNEQERQINPLGFQITYYRADDEFL

SEQ ID NO: 19 - protein, 208 aa, Ehrlichia chaffeensis
MNKKLLRRYCITKISVCVILLVVLSMTLHYLYKFNTYIVQHNLRIADEIQKLSFKIMDVHKHEAMLNDSGVLWQEISTSNIYSTFYEEDLGTLISSLFKKYYIFNSQINISSPKIVNNIYHNQHIDIIKRNIEIKFSSISDEQVFLFLNTIRHDISGYVKVVNFNIEKNTDITNEVLQAALKGETVPVIRATILFELYNIVGRFVDES

SEQ ID NO: 20 - protein, 357 aa, Ehrlichia chaffeensis
MKYFNKKLIVISSIVIASISSLYIGVWLVIATHVKATLSKSLAYIHAEHSNDIQITNFPFIPKIKVLNLKINSSSLNISIPSLSLKYHLLSNTLEFIGLDNATLTFENSMKITNNDQKNTSVCNLHDNFKLLIKPSKNLLLFLLDNKNDKKAYFTSMVYEDNGISCNNNHIINKSIFSVETDEHKSFDNLITVLQYKINASAASNTDTNINVKIKDAIVTLSMSNTEFKEISAIIDNIALTSRNSLISIYGKLLLPLSLDTQINDEKLIIEISNYQDIIKQLVKILYPKSQHKDKIFSALQDYIYAISEKKDNGNIILSVSETVNPFNIFICKVPYEEFISKIKAITNTMDNHANKK

SEQ ID NO: 21 - protein, 221 aa, Ehrlichia chaffeensis
MNIINVRRELSEQSKKDNSALSDARKQIAVACGFIGESTGFLGIASILLILGSHLLPFSSIALPLRLFNIVASLFSVTYGSILLMKSIEGNARSKQCNSVRIRLHAVWSLLNSVIFVSLGIYNLLYETLLHEVLDFSANHDLAMLLKAALFIGYAVILTSIFLSQVFVPSHDPVANKSSNTLESETATLLGQEGCEELCATLLEVDPINVNRGELDNNRCC

SEQ ID NO: 22 - protein, 1963 aa, Ehrlichia chaffeensis
MKVWCYKNIGLYLIVLLSFVYPRALLHAKINIHVLRDYANVHYNTYYGLHFDNYYKPVDNTEGNLDSVMIRFASHDSYRYMSFMQYVKGQYQDVENLNVSGLDIPFNREDRTFDDHDVMFMQLSSYWGKVTFHSLPIGGCKVLYAGSIIYDPVSAIAFLENENASSKVCICFVGKCNVKPERNSCDKKSIRCKKVTVAINPPPFCSILDASSIVSITPLRFSQQTFFRPGVRIHIYSGSGNPYTKELYVKSHKIGDKTSYNISHAGIPYEFQVYKAGYDTVCAEYSNNGEKGKKVCVPSPGLMRPKVTSNANGVNIQYQDCQGLSSCSVDMLPGTQDLDMYFSVIKPKIDFNNYTLLSRYECEDGKIVENENQCVNGIGQRLGYVHDNNSNVTCVVDMPFVPMKYSIKKNHRDLWLSMHDKMLLGYGVVVGKTDTGKDVERYVQCDQKFAIDIKSMTQEQLNKITRIRQDAFFDIGGHYNPKNAPCQDSMLYRYENNRLYEKGGQVSCKNMVELDYGTKKIKGCSSLYMSDDDFTYFFHENDELEKIVPLNPILQGMCVSNFPSYEYKKRVLVRKILPDSYKLGIDQKNTECDFLKIEAWGGGASGISRSGRSGKAGNYVMGLLRFDKNVVNKKLIIDIGDGGKGANSLSNSGGDTTVKLCDDDDKNCLVKIIAHGGDEGGNYLQDSSEGIDNLVHYRFAPGLQNSGESEILVPYQSPDMPYGKLRKGDKECLCDSNILEKNSNKYWGAGGCSSVYNCAQEGANGMVRITCEKWSGNVGKISLIDENACSDFLVTLIEKMHKSTSGIPNVVKEFLQKISKVSFCRQSKSFPNLISSMSKYFIAIDKILVGGDILGHNLSGLRKELFTELNNTEVKAMLAKLGINESPETLLLYLDVLNFNFGVNISNPPSGLLNYYVSDNEFDYDLSKHDEDYDKLSDNEAMMFNVTTEKPEQWFSVELKDPEFVRRYRNFINMIHRSVITDEEGHKKNNIVVSWMYTFFKSDRQLFELYAAPFVELMLGMDLNKFMKWGNCSDTHIRLFESIGKYSERLPSQIQDFIKKIATGDFCNTFSKMELLNSYGVELSNYAIDCALKNTDKRCLESKWRSSLGSMAKKLQDAVDLNYEVFTDIGIASNRKEIALLIDAVMLNYVMSDLNVGNSDITSTLSLLDPISSQALDNFPYDTIVELRQDASNMKYGKYGDHRANIVTLWSYMAYNSFEWNIEDFKSFVKLLLAEDLTSVKIRKCNDNLRYLFDKLNKYKSKLPAVLQDFLDKISKESVCKKISRFAALETSLVKYEENLLNELRGGNLFYFSSLYDLNYAHRGKLSNIEKVYSHAANIWSDVGELSSNITNLLNNPEIYKIFTDAGITSSKEEISLAFDAVIFNSLVSEIKIDQKKLKNLLLLIYDNSLALNNIRLERSKGSGQIQQVTIDQNRYPGNGILMLQQNANNMEYGNYGVHRADIIALWSYISYMSSESGWSIKKCESFVKLLLGIDLKFIDLKGCDNDVVDLFNKLNTYGDKLPLSLRDFLKKISEKNFCEKMSLFPELGIALMNYTNELRNVLRVGSVFQVDSVANIVNGRVSNINDVYSYLGNLLSNVRQLSSNIADLLNNPDIYKIFTDVGITSSQEAIFLSIDAVIFNLLVSEIKIDNSQLKELLSLVGGHRNASSNNANNGRSLSQGIRYKITFKISFAQNYFPVDEIIKLQQDANNMEYGVHGVHRTDIIALWSYISYASSKSKWLFKRYQSFAGLLFEIGAWGKCTSAERVFFTSMNRYSEKLPLKVYNFIRKITTGDFARKFSGMQSLYTYKQRVYDYVMHCVLRGGLGGECSDMTLREISNELHKLKQEIYSNYDVFRDLGITNGQQEVLLLINVMMLNYAMSDLLVSTTQVNSMLADVRSSLSRTAHCLPYGTVSQLQRSVSHMKYGEYSNYRASVVALWACISCLASFNDDMEPLIKLMLEGD

SEQ ID NO: 23 - protein, 205 aa, Ehrlichia canis
MKAIKFILNVCLLFAAIFLGYSYITKQGIFQTKHHDTPNTTIPNEDGIQSSFSLINQDGKTVTSQDFLGKHMLVLFGFSACKSICPAELGLVSEALAQLGNNADKLQVIFITIDPKNDTVEKLKEFHEHFDSRIQMLTGNTEDINQIIKNYKIYVGQADKDHQINHSAIMYLIDKKGSYLSHFIPDLKSQENQVDKLLSLVKQYL

SEQ ID NO: 24 - protein, 347 aa, Ehrlichia canis
MMRLLACLGIVAVIIVAFNFLTNKQQVQDQKQEVHVYSSRKEELLHSLFEQFTKETGIKVKYINDEAAQLINRMENEGTATSADVFLTADAVNLILAKKKGLLQPIQSEVLNQAIPSKYRDSEGFWFGLTKRARVIVYNKDLVEDNELSTYEHLANTKWKDKILVRSSSSPYNQSLIAFMVANNGVENTKIWVKGLVANMARKPSGGDIDQIYAVAADEGSIAIVNSYYFGRIAASDKKSDQAVVKKLGIFFPNQETTGTMINISGGAVTKHAKNKQNAIKLLEFLTSVRAQKVYAQVNQEYPIVEGVELSEVLKTFGTFKESDLPLQELEKHLTKSVEIADECGWR

SEQ ID NO: 25 - protein, 328 aa, Ehrlichia canis
MVLQNDNITDDTQNNKDEVHDTQQSTEIVKESSVVTSETQPTHDKKLDKIDFNTLFLAISFAGLLIEAASAIFNLVSTYVYVPERIKHIVATAFYAISIIVSLSMMASSILAIKQSLNSKKKLQETSTGPNKEAERSINEGLLGYDKLKQKQANIQISENTITIISEILWIIVSAASLVMITVGTGTPALELASLCLAVIAPFLAFISCALRLSDANISRKTATSNKEKRHASNFTVLCTIILLFEAIHCGCHVAEAVMLGGKMQNIYDFQDAIVLGLELAAVVMFIAAFFIEKYLDKKAEKSDPQATPSSLLDDKAIDRMFREAQIS

SEQ ID NO: 26 - protein, 395 aa, Ehrlichia canis
MVEERKPHINVGTIGHVDHGKTTLTAALTTVLAKRLSGEGNKSVKYDEIDKAPEEKARGITISTAHVEYETENRHYAHVDCPGHADYIKNMITGAAQMDAAILVVSATDGAMPQTREHILLAKQVGVKDIVVWMNKCDVVDDEEMLSLVEMEIRELLSKYGYPGDDIDVVRGSAVKALEEETGSGVWSEKIMELMNALEKISLPVREKDKPFLMSIEDVFSIPGRGTVVTGRIERGVIRVGDKIEIVGLREIQSTVCTGVEMFHKALDAGEAGDNAGILLRGIKKEDVERGQVLSAPGQIHSYKRFKAEVYILKKEEGGRHTPFFSNYQPQFYVRTTDVTGNIKLPEGVEMVMPGDNINIEVSLDKPVAIDQGLRFAIREGGRTVGSGIITEILE

SEQ ID NO: 27 - protein, 634 aa, Ehrlichia canis
MAVIGIDLGTTNSCVAVMEGGDAKAIENSEGARTTPSIVAFTDSERLVGDPAKRQATTNAKNTIYASKRLIGRRYQDVKDIKSSYEVVSAKNGDAWIKVLGKEYSPSQIGAFVLEKMKETAERHLGHKVEKAVITVPAYFNDAQRQATKDAGKIAGLDVIRIINEPTAAALAYGLNKSDKQKVIAVYDLGGGTFDVSILEIADGVFEVKATNGDTMLGGEDFDHAIMDYLMDDFKKTTGIDLHNDAMAVQRIKEASEKAKIELSNRMETDINLPFISSDSTGPKHLSLKLTRAKFENLVDDLIQRTIEPCKKALKDAGISADKIDEVVLVGGMTRVPKVIQKVKEFFGREPHKGVNPDEVVAIGAAIQGSILAGDVRDVLLLDVTPLSLGIETLGGVFTPLIERNTTIPTKKSQVFSTAEDGQTAVTIKVYQGERKMAADNKLLGQFSLEGIPSAPRGMPQIEVTFDIDANGIVHVSAKDKASGKEQAIKIQSSGGLSDDEIQRMIKEAEQKAGEDEKRKKFIELKNNGENLVHSTEKSLNEYGDKIPNSDRLEIENAIRDVRDALGNSDVESVDILQQKVDHLMKVSMKLGEALYGNANNTSSTESTTTNNNNEEDSKVVDSDYQEIDKKDGK

SEQ ID NO: 28 - protein, 400 aa, Ehrlichia canis
MSEIQVKAENLGGESILEAPIRVSVNVGDTVKQGDMLFIIETDKTSLEIVSPEDGVIGEIFVTDEAMIQRGQVLCTINTVQSSAVESSDTSSAHNATTTAADCMQQFIQKKDAPSATKLMKENSIDRDQISGSGVSGRITKSDVLNYMKSTASEGSNINRLAVVSEGKREDRVKMSKIRQVIAARLKESQNTAAILTTFNEVDMKNVMDLRAQYRENFEKKYSIKLGFMSFFIKAVILALKELPVINAEISGNEIVYKHYYDIGIAVGTDKGLVVPVIRDADKMSFSELELTLAALGKKAREGKLEVSDMAGATFTITNGGVYGSLLSTPIINPPQSGILGMHSIQKRPVAVDDKTIEIRPMMYIALSYDHRIVDGQGAVTFLVRVKQYIEDPSRMFLEV

SEQ ID NO: 29 - protein, 630 aa, Ehrlichia canis
MKSKGLFVKPLLIILVCSLVFIAFGTSFFPGNFNNDKYVAKIGHEKLSLQDYTNAYHDELRYIQQILNRPLTEEQIAQFNIKLSVLNKLIENKVLTKFTDSLNLKVGEKSILSHIKSIKFFQDENGNFDKTKFNIGLSNAGLTERLYINKLEKAFPVAMLMSCLFSGTQNTYVKYHPELIKQILQNLHQARTIDLIEISPSTNDISVPSLDELKKLYEEKRKSGNLTFPENRVIEYLTINYKNFINQINVTQEDIDNEIKTEELDEQRDILNLVFSTKNEAEAALKALNEGTDFNDVVTNIAHTTTDNITLNNIVKNTLPNDIRGKVFSLKEGDISPILHSMFGWHIIKIKSIHKISTEDLQELSEKIAQNIKKKKAISLLTTEIESINDKINNGASINEIAKLYNLSIKTINTDISGKDLSGNKIKMSESNADAIILAAFSAQLNKPSNFTDNDDYFFSVNVTKVEPSREKTFDESKEQLVYEWQNDLKSQKLSKLTQEVVVKLKSGIDIHNIDGVLLNSNQMVYRNSVAAVDNPGQNYPTDLVDEIFNLKKGEISKSYQSSTNSSEQKMLIAILKDIKNADQVSNVDFKNIQEKIANDNLESLKNQLITYLTKKYSVKINQNLIDNVR

SEQ ID NO: 30 - protein, 425 aa, Ehrlichia canis
MLGKDYFSDLDKRLVAFAKLNNGKQRSSKYHFMLSSCILITAIITVLSLIVLYSDKQYFSSMFKGGFKLFSSDTIPFSMALIAIIPSLLLLFFLIYKVCAFHDLNRKLNNESIDILGKLEEWQYFLHLQLEYNTNKIENIDEVLKLFKKKYSSFVKQCSDGFAKLEEQCKSVLAAVNSTTHGVNAGISKINCVVEDVKLQLNGLSAGCQKFSESSSNLLSAIESAIKTEGVNVDDRIAFLQNLQANLASDVDLGLIQNRVQNFVARVRSILVEKPSIVKPAVLRELWCLQESFNVIIMKVHKTDQCALEFLQLIQNLEQEISKFSLSASKKIASYNGNNRNLLEKMIKLELPLLHINILRFLTAKRAAFITNTSADITQFIQEYASGDLQRLSDSTSDSMLSISSFLSLQDTNVSDKVSNDINPQ

SEQ ID NO: 31 - protein, 182 aa, Ehrlichia canis
MGNLDIQKYVYLALFLLLLVILLMLAIYRYCVASEHDLQRQDVLVLSREYYNQLAGGYRQLKNDQSVMFVHNVRLAKTCEAVAEEKRNLEKKIKELGDDMSEVSVSMAVELNSLGVHPTVVSSMTSQMLSLQRRIYDVARSSPVSIVKADQGVQTEQQQASSALDQILASGLQCNTGGKVVH

SEQ ID NO: 32 - protein, 194 aa, Ehrlichia canis
MNILSVDNVQDLRNLHAISHPIEKIDQEIIALANDMMKVMEDSKTVGLSAVQLGNHSRMFTINMFSGLFDVTQDIKVLSGHHSLHGKNMVCINPEVLSFSAETVDLFEGCSSAKSYGLINITRPKHMDFRYTDLLGNKCVVRVYGWLSRCIQHELDHLNGILLANVVDNIKNNCVNSISYEDHSVIHILLVNKK

SEQ ID NO: 33 - protein, 372 aa, Ehrlichia chaffeensis
MIFDEDSNSVTQDSGYMTVGNAYSNAVGYISMSDHWSKLTKPSSIKVESNGASPNKADLIVEPLESGFALTLGNALRRVMMSSLRGFAVYGVEIENVLHEFTSISGVREDVTDILLNISMMRVKLSGLSNKVLSLRVKGPCEVRSGMIPDTDDCIILNKDLLICTLDQDVDFNIKMYVNSGKGYVPAVKRKSVSKLSDVPVNFIATNALYSPIKKASFKVESSRIGQFTDYDRLVLSVETDGSILPDEAVALAARILQDQFQPFINFDETDEPHKKIDTKDALPYDSNLLRKVDELELSVRSYNCLKNDNITYIGDLVQKTESDMLRTPNFGRKSLNEINELLASMNLHLGMKIANWPPESIESLSKQYSEE

SEQ ID NO: 34 - protein, 492 aa, Ehrlichia chaffeensis
MFVKLKLNDDLESNTTTTSDNTHDNQDHNQTCATKSKNIISVTATEDETTSDDSYNTQELTPQQITQALDRFIIGQADAKRAVAIALRNRWRRNRVPEPLREEIIPKNILMIGHTGIGKTEIARRLAKLAKAPFIKVEATKFTEIGYVGRDVDSIIRDLVDVAINLVKEKFRKIVEKKAKALSESMILDALIGPDASEETKTIFQEKLRNGEFEDSEISISIKESKNTMPPIDIPNIPGNQVGIMNINEIVHKMLGNNKQLKTIKVTVKEARELLINEESEKLMDEDKIIKEALQLASNDGIVFLDEIDKIAARTEIRGEVNREGVQRDLLPLLEGTSVTTKYGTITTDHILFIASGAFHLAKPSDLLPELQGRLPIRVELKPLSKDDLVRILTEPESSLLKQYCALMKTENITIDFTDEGVCTIAEIASTVNREVENIGARRLHTILEKLMEDISYTATENSGKTYVIDSEYVKQKLEDISKQLDLSKFIL

SEQ ID NO: 35 - protein, 225 aa, Ehrlichia canis
MVLFMKAHSTSIRNFQPLERAAIIIAVLGLAAFLFAAAACSDRFQRLQLTNPFVIAGMVGLAVLLVASLTAALSICLTKSKQVTQHAIRHRFGYESSTSSSVLLAISIISLLLAAAFCGKIMGNDNPDLFFSKMQELSNPLVVAAIVAVSVFLLSFVMYAAKNIISPDKQTHVIILSNQQTIEEAKVDQGMNILSAVLPAAGIDIMTIASCDILAVSSRGSSQHQ

SEQ ID NO: 36 - protein, 228 aa, Ehrlichia canis
MDMNKGLLVAIAVLALFVLLLLFALYIAYRKYKVCSNQMNCLSEELIVGEKRYNTALGKLEKEVHCLTKQLAALRNENAKMLLALEQSEKSEVAFEHDSALERQLQLSNKQMVLKNCAKLKDLASRLRTIVSEAQARHEDDVMQIEHIISAAFNSMVSCYKSMLGNSCHQQVKIAMKDLESSIRCVKKHLYVLCDKLGEGFISAEEDISDEVFNPVIQIVNHAQKKVA

SEQ ID NO: 37 - protein, 451 aa, Ehrlichia canis
MRNILCYTLILIFFSFNTYANDLNINIKEATTKNKIHYLYVEHHNLPTISLKFAFKKAGYAYDAFDKQGLAYFTSKILNEGSKNNYALSFAQQLEGKGIDLKFDIDLDNFYISLKTLSENFEEALVLLSDCIFNTVTDQEIFNRIIAEQIAHVKSLYSAPEFIATTEMNHAIFKGHPYSNKVYGTLNTINNINQEDVALYIKNSFDKEQIVISAAGDVDPTQLSNLLDKYILSKLPSGNNKNTIPDTTVNREDTLLYVQRDVPQSVIMFATDTVPYHSKDYHASNLFNTMLGGLSLNSILMIELRDKLGLTYHSSSSLSNMNHSNVLFGTIFTDNTTVTKCISVLTDIIEHIKKYGVDEDTFAIAKSSITNSFILSMLNNNNVSEILLSLQLHDLDPSYINKYNSYYKAITIEEVNKIAKKILSNELVIIEVGKNNNINGKQIDAKKHILG

SEQ ID NO: 38 - protein, 243 aa, Ehrlichia canis
MDNYCIIICAALVILLAILMAVLFGLLYKSTRDLKRSLESQSKSYNSEIGLLKQSLESLKANIAGLNLQNINSVLNRVNTYDKKLEIIKNDIKGFSSKCNSLETACDTLKKQLISAHGELASLSKKIHEAWKDKNKDGTVAIPSQNQIFDIDLMALVEELNIMKMAVAKLMSQNETHDLMNLEHSVSNLVCSVSSLSDSLSALESERVEYSIYIKNIAAKVASNLDEITFRVSAIERKISDLH

SEQ ID NO: 39 - protein, 211 aa, Ehrlichia canis
MKTSSDNSMIVTRMKYQMDKMGINARELAHKADVGKSFVYDILSGKSTNPTSKKLMAIAKVLNVSLSYLISDDNYTYGQGNMNILPVYDLELENGKISSSGDVNLYLSSNINLTSNMKNLRVYYVKGDSMIPTLMNQDVVLVDIGDKIPHPAGLFVIVDTVGVSIRRLEYLRENQKIKLHVVSDNKKYSSYECHLEDMEILGRVIWYARSL

SEQ ID NO: 40 - protein, 527 aa, Ehrlichia canis
MPNNNDYDTYSKFIRDIRSTVFYIRNRGVLHLGDLVSNVTPATYIIDSAKEGIREQFLSRVFVHHSNNKNDIKKVIDFIISLGFIPVVNADYKVTEHEGVCIYDKNRKYGDKHVLIVSSVVRHTTRDVRNIILESRKNFRSSFDDGAKDSIRTVLYDPDSKPLKQRIYDAISYSEKWSLLTTQEKVIRCIFTVLSLSIPLIPPLLLLSYIGNIDIREAAYSMNLLPWQYRVYMKTSDSLYDVIVNNSDEERRKMITDNIMETAMAKGFVVKGEGDEIQPTDLGKHVSGKHNLLLVAHNKELSKKQLLLRITTFSVLAFVCFSQIIALTCHLVRAEKIALYCSILSYFVIFVEGLYLLSLKTTMSKVCATFCLVFAGFMLMVHAFLLYNDITFGYGPIMSGLMVIITGAVLSVALACNQYYKQDRDLALKLGGISTTAYLSMKRDIDGIDEVKTVKLEGGCGYQIIDDLSLALFNAYNAGVFGTVQDQTVGNRQDEIYSGTQENDNCPNTSIGDLQSIDRSNSQDLNL

SEQ ID NO: 41 - protein, 120 aa, Ehrlichia chaffeensis
MDMINIFDNTEDDAFSVSNFINQNFISQFTITILPPSVPLYHDQHIDEGMYSVVFSYKKYEAQQPYGLVEHKSGKFEASLDHSDHRLYLNKDDISIVLNEDMLNLCLSCTKVIDNKDSAQ

SEQ ID NO: 42 - protein, 671 aa, Ehrlichia canis
MTSNTSIPKNHEYSFKLDIGENLYFFCNHNVHKVKIITEDNTEITMPSKNYFFVGDKFYAPYNNYFYDNYLNIPAEYRYIKVDHMQYRTTNNEQPQDFYNLVLCDKNGEEYRYNYYKFYIKPENIIEKSAEINLKEYYNIQQLKEGAPLFKIVSEQPNNTTKASTALILDISSNQKFAKLSPEALQYKHYLDRNSPTYDTFTLSYSDIRKHHVDEQEKINLHNIRDDILQAEMENNPIFLVIQDGKYFFTDVKQDQPLTTSYNTALKVLASANFQINNVPNDNCYVDMHKKFIFKITKSNLHTEHDNSKNLASITLEGKEIPLISNDDDTQIFYDDFSFKCYQNFTQVFNYDEPIIGLDKDFYEPIKEKLSSNNIYITIKSDEQNHIKTYFSDKQGNHILDLPNTKLTEYLSTMLPLGDFSNEVLNTHIEDIAHQKLSDTTQKHDTLNPEKNSTTLQNSVNETAGTNDPQSTQNAVHKHDTLDTQKDSTTSQKSVNDTASTNDSQSTQNAVHKHDTLDTQKDSTTSQKSVNETASTNDPQPTQNAVHKHDTLDTQKDSTTLQKLVSEEHNINKSNTNINVEQNIVYFPLSREHVSIVDNIEQNKHHVSFNLTYEEMLNFYEAVKEQYSYDEVLIAYNNIFKNYGREQKNDNIYIDGDNHIFIENHDFGILQ

SEQ ID NO: 43 - protein, 182 aa, Ehrlichia canis
MDIFSNELNATVHVNGTTYEGKVIIDNNGNFDTNLSLADGVDTLGHLCGNISQNNETKENSYILEYIFEQRIVYPTLPILHSFNGQIVSSAEEALPHQIAFDNSNDNIKIILSDSEIVQPVTNAKESQAEVSKPVTDVKENQDGAPQPAANTPQEKQESVPTPADGVNNDPTKEGASQPNKT

SEQ ID NO: 44 - protein, 368 aa, Ehrlichia canis
MFGFLKKGASVIIKAAVTPTTSKLPHQESVGKHLEGMLKSIVPGQKSSREKNFDLKMHDRTYKLGVELPGRTRVAGHETEVALKVPSYKLPYQALQKFAAWQEQNKEGEVDETRKDALASLITPVAATRELIASKTADQYFQKLKELDSQLKIICGKVTLDSTAGSYKQGLTVELDVSGKSKEQIEQEVKIVLQSLGITDSKLAKTFAQRLYKTSTDPKVSSADIQDKKTNTDSKDTTDDKDVVSSAPAQNSDVVSPKKEQRSDLYPAPESSTMSDSDFSLTNPRNMPYNTRGPLQSSEMLQKLQQQILMQHRPEPLFPQPMSPSMQQAVEEIARDLPTNINEEEHMQGNSTNTGIPIHNTGKAHARR

SEQ ID NO: 45 - protein, 133 aa, Ehrlichia canis
MSIDYDSCQIDFSIQRSGHPGVDVYHGIMHVNEEGSSYYASGHIVNDSGNEGIVNIIKYCNRGIYNDEHCTECDLEVILDDPNSSRYVFHVSKDDTCLMCDERNILSMMKPSRKVQIQNDAVVALDIYGCKNT

SEQ ID NO: 46 - protein, 329 aa, Ehrlichia canis
MFYPKRDLKTIYSKDNTLSAEHLLELMNSSSKELKEYRSQLLTGYVKKEEKKLTDQNKTPEEIQKHISSPEFFYKASSYADYKVNHGIEMFLISQIGNKVKILNQRIKSLGYDRQSMILHGMYKISLLTEINSIIKDMSDDDFKELLEVIISCSHLKTDLTEQQQQSRQAAVAEYVKESKGFSMEKVREKAEELTGIDIANPIASLRKKFGADKPEGKQHMVSMDEKLVQTVINPFNSYYFILGALAIGLEGFIPPLVPILLIAITGKSLFSLVTEDKDKEGSVHSPFQEGIIRQPVTAEGQQNYTAMLERQKKEESEQQSTSPTSKKL

SEQ ID NO: 47 - protein, 91 aa, Ehrlichia canis
MGNSGISIIVCVVFIFLIMMVLVSMFQRNASIFDCVSRQQCKASNADHDKGNDTSNQSEEASVNTDLVSGSNGTCEDIDLQSVQSRKCCRT

SEQ ID NO: 48 - protein, 194 aa, Ehrlichia canis
MVALYTPQVDKNVIAHDFSLKSTDGNLYSLSSYHKGNGLLIMFICNHCPYVKAIINNLVYDVNTLKNDYNIYTIAIMPNDTIAYPDDSYENMIKFAQEYGINFPYLIDEDQTVAKNYGAVCTPDFFGFNNKLELCYRGRFDSSGKNQVDSKQEDRDLFNAMKFISKTGKAPDNQKPSIGCSIKWKSQDDQFVPH

SEQ ID NO: 49 - protein, 165 aa, Ehrlichia canis
MCIVMLMLLLLIAMCQCCISMRTLDGKFESEAMAEQRIQELVDENQNLVLENRELSAACKSANGKLSELLLQMKNLLLSAETQSEKILNDLALDNQANLCTKKAVQCGVLKIRQGIFDIVHNNREEQRRNIELGGNTPSKIPVSKLRMQSLSTLQSENKVQSCCN

SEQ ID NO: 50 - protein, 188 aa, Ehrlichia canis
MDTTAIVLLVLLIVMLLLLVLLAVAAVKCQKQYNFLKEENKKLLACCEEASDYSQELVSQNDKLKKESERIKSKSDKLERELKNKMQKFFSDQVSEYEDLKESCIELINIRGESVKKLFDDVKQDMSPESVKCLIDTNILFLQKSITYEIDSSKNTLEKRLSDQFPSNDIEGASVSSTGGNVQPNDIA

SEQ ID NO: 51 - protein, 226 aa, Ehrlichia canis
MLGDEIAAIVIGVLLIIVSAILLYRLICWMLTPELHVKKGIPSHIDVQQTKFDSEETKPQTVTLPKVRTAGYLQFMLEAHDCCFNIVLDMMKQSANNKMLVGDNLTFLMDKICNSSSKLKELQNLSIGSEAALEQYLPTIYQCCLALVKYGSLIQSQIKKVSRKTCNTEQLIPYQLTVSVLMHECNMLLREIKQEMVNATESDRLPVSILESSTANSHSTSTGLNQ

SEQ ID NO: 52 - protein, 121 aa, Ehrlichia canis
MYRDIFCIIVAVAVVLVILILLCIIGYMISCIKSLGNQIDKMKTELSQCRKEDTEMMEEVLTLYDEVYDMRNDLAYHPYFGNVKNGKKLGNEVGDTTLQQGSCTLDSTNVKGLGDKCEKDK

SEQ ID NO: 53 - protein, 535 aa, Ehrlichia canis
MNKSQSLYLFLFLLCVTILAFTAAILAFRFNDRLKIPYLNIIAAVVMSVLAVALLISVAHLVMHYQGTSVSVKDMPPRIAAGDQMVLFVPFTADQLSKLSPKNNIIDVKYDVVESGSSLAGKREFVLDITQSYGLCPTKKVQVVMDNNRQCQLLFTNEEISSQVNGPGMCFVVNGNTSIVKGNMQKPGFYIKHPEVKDGTLSLNCEYQKICTGSMEDLRYILRSECVRSGRTIYEMLLSNILTMCPNQKDKVIPSDASVTHSKDLWINTNSGRLALSLMKIISPDSTLINLEELKNDDEPLYQKITWIRNYLVTNGHNVSEASFEVIFKKASDIFYRRTAGMSEYGNLHSDRITLSGKKFSDILAEGMQLDEELLIIMAIASYSCEGTLDPYNTTNKFLREILMLDQQRSDSILKEIEEILGKHWYEIIYLNDLKFSGSKVCTQMKKLLQDGCDSATALEIVNAARAYMLEKQWSSVPITRGVGCIMNLGYLDRIRLCGNREVSVETQEEGVSSSIVEVVDTSSVQNLLAPGNMQ

SEQ ID NO: 54 - protein, 229 aa, Ehrlichia canis
MCILGADKIIPPHIPAVGTGKGYSSDGIAIPRNSKEILKNKITPGTKPLVVFIGGALDDTTRLVLRLYARYYTQNHDHQDIAYATWGSSATIPIIKAWYEAEQKICLVGHSWGGDSEYKIVKKLEMNTIDLMCTIDPVSRAGVGGKLPKPKNLKKWYNVAIDYKKVKFSINNLIAQTGGPWFYCMYADKNFIVDTVEINKHLVSADHAMAEALFFKYFNEYVKNFATQE

SEQ ID NO: 55 - protein, 98 aa, Ehrlichia canis
MADDEYKGVIQQYINTVKEIVSDSKTFDQMFESVVKIQERVMEANAQNDDGSQVKRIGSSTSDSISDSQYKELIEELKVIKKRLLRLEHKVLKPKEGA

SEQ ID NO: 56 - protein, 354 aa, Ehrlichia canis
MEVNYKTIIGIVIGIAIAALITIAVLTVLNVIPIAVLLQLSTVFASAIAGSVVVYAKEKIFAWYDKQQYEDRASTSFKMVDKVLSASDEKIAKSSDKVVLSNDTISNYSNYPFFPEFHKNFNKILDKLSLDVRFASINQELFYSSLSPQEQTLAILSARLICKQPEMYLIYSEPVRVCIAAIAQLKIDKKFEGRARNNIISLLRKQVRISNYNVLEQELKGVMLKHTKDPVFMKLLHGLIDIDEFSGKKQIMNLVNLFSSYKYYSIYSDSAVEYYSDMASELLKQINNKQESAVDVDAKSSNVPKHKTSDNEKSIVDKRTSNSSVNACDTNNQELENNFSSKITRINSTKMRGL

SEQ ID NO: 57 - protein, 248 aa, Ehrlichia canis
MCERSLNVIIALLALLALLIVVLLCAVIYKCHVDCQNERQRSSEIEEMKLLLRGQPQAILEPDVDREKIEKLCEEKFKNQLKEQQKANSDLQSKLKSLEKTNKHLAEVVSSLKKHGVIINRKNTELTEQCSMLRKNCAQLGVVCRKLGYDNKVLMRDAQVVFNAGFYSFKEAVDGMLQISAEIELLLLKRNERIDALTVGEIEYHLLTLRTILMDLEKQEKKSQKQPTQQVKAASVLPCSDQDNNKAA

SEQ ID NO: 58 - protein, 507 aa, Ehrlichia chaffeensis
MLIDQKFQHLSQAFNSYSTLYQRNKLKSKYNFLLGSSIYLTALVSVFSIVALYHHSGCFNSIFSGKDKLFGGNIPVVFSIALIVAIPSFILLFFLIYKICATQDITDHVVERTNDLIGSAREVCEFLKEKLHENTCQVEALVGHYGSLAAKHDDFVKKCSRCLQSLEEQQVALGALVRRSTSVVDNNISEVQALVVDLKAKIAEMSVAHVKLMDASEDVREFIVKQLRLEGLGLQHRVRFLDGLQGTLSQNIDVSDAKSAMKIIIERIENEIPKVRFAFNSLNSMLWSLHSNLLKALLGLDKDSSPDGCITVCQELVNALHKFLGDMRAKIDNYNGSDKRQFVNIGLVEVPLLQIMILRLMATKRACMFEDVCASVKDFIESGTADRSSSTGTKVIPDSAASAAAPVTPTASVTPAASVTPATPTTPAASVTPIASATPATPAASVTPAASVTPTASATPATPAAGTNDGAEFATPPMGTTDAPSVEDLSALGAVGGVDPNAHTHTP

SEQ ID NO: 59 - protein, 205 aa, Ehrlichia chaffeensis
MRDKVKDLSNNNNSRNSNPIWNILRSIFTTLGNWGNMLANVLPRILISGIVPPIIPTNATNNNPQHNENSSTTHSNENTQSSDHNNMEQHTSQSEIPVLQSSANLDSLSDIEETPRRNNEDEIPVWESSTDSDGISDTENMPIDSHEQHDSDTVSNDTNSDIEFISEEDVLEASGFFIVDICDNPNSSVTHIDLQSGSQDMVYRP

SEQ ID NO: 60 - protein, 175 aa, Ehrlichia chaffeensis
MIHNGNKKHSATNALLNVATVVSALVMTFSAFCTIYSGLNLAFNIKIPGTPQTSMGLFLAFVLTFTAGSLSLISAAVANRLSQPFVLTQETSRNTSQQNTSEDDQHRLRSTSQQNTSEDDQHRLRNTSQQNTSEDDDQHRLRNTSELDGQDNNSPPSSVVRQSTMQGIPSAQQEV

SEQ ID NO: 61 - protein, 181 aa, Ehrlichia chaffeensis
MLSLLIYVMSKKCYMTAAITVGEGEDIQSVQAPERNLSNGVSNPRVVNSIDSSSNMSADSASDMGADSASNMSADGAGNMGADGAGNMSADGAGNMSADGAGNMSADGAGNMSADGAGNMGTDGADSGSDIDTDNSIGSDVIEGGTSGDEEEELHSVDQHMVNVGNNGVIAVEPQSSICFR

SEQ ID NO: 62 - protein, 193 aa, Ehrlichia chaffeensis
MVALNTPPVNKKFIAKDFLLKSIDGNLYNLSSCHKGNGLLIMFICNHCPYVKAIINNLVYDVNTLKNDYNIYTVAIMPNDTITYPEDSFDNMINFAQEHSIDFPYLIDENQTVARNYGAVCTPDFFGFNNKLELCYRGRFDASGKNQMNSKQEDRDLFNAMKLISKTGESPENQKPSIGCSIKWKSQDYKFVT

SEQ ID NO: 63 - protein, 142 aa, Ehrlichia chaffeensis
MDIFGNEFDVHVNANGTEYAGKMSIDSNGDFDVNLDLQDGVGTLGHLSGHISQSDDAANYIIEYIFDQCIIYPELPVLHSFNGQIVSPAEEGSIIFDNGDNIQISLHGLQEQPEEAIPAAEVEEAVPAEAAIPAAEVTENQQ

SEQ ID NO: 64 - protein, 118 aa, Ehrlichia chaffeensis
MNNTNITNSTQSNNSTNSDNSVTIYAPIIAITCLFILMIIICRRMHRNRENQVSTTAPSRSTSFEIRRHTNFTNYVLYDSFYEPELTDREVMALVDVDAPHNNTRRHNNSNSTNASGR

SEQ ID NO: 65 - protein, 248 aa, Ehrlichia chaffeensis
MAGHSQFANIKHRKGAQDAKRAQKFTKLIREIIVAAKQGLPDPEFNSRLRSAIIAAKKENLPKDRIDAAIKSATGNTQTDNYEEVVYEGYGPGNIALMIQTLTNNRNRTAAELRHALSKYNGKLGESGSVSFLFNHVGVIAYKASSIDSFDSLFNTAIELHALDVEEIIQDDTQEKIYYVICNVQDFGNVRDQLFHKFSDAEFSRLFWKASDTVKIEDEEIQKKISNLMDLLENNDDVQYIDSNFTFY

SEQ ID NO: 66 - protein, 179 aa, Ehrlichia chaffeensis
MSEGLIVVIALQVFLLLLLFIFALYHYCIKPCSKSGKMGYERFEGENIPKYEGVLEHFEEMDYLQLQQRVAELRLACRMLDKERVTNDRTYEECLDWMFFVSLTLDELMVENKLESDYITRMRAQYHLFNLRIIADQVARQPAANVERQEDLQEVEQSPSPQVEGTSVASCCGQEKQAA

SEQ ID NO: 67 - protein, 172 aa, Ehrlichia chaffeensis
MRNVKYVLFWLGFIVLVMLFSGTKDDIGGNKYFNKFFPNAATQNNKVNYVEGGVEFYRAKDGHFYIEAMIHGIPVNFLVDTGATDVVLSVEDAKRLKHHLKYLNKKKTYHTANGTVKALYVEISEMQVGKFVVNNVKASVNVSPMRTSLLGMSFLQYFHFNMSGDKLTLHSY

SEQ ID NO: 68 - protein, 551 aa, Ehrlichia chaffeensis
MNKTQSLCLLLTCFLFFTAAALVYYFRKDISTTPYLHVAAILLMVLMAIMAIAMLVIVAHMAMMCSQSYTKIQSNLPSIKEGKEVLVFVPLTEDTVSRLNFGGGDIFKIECNTRASVMNLGQGNGKGIVLTLTRKYGLCSQRTVDVQVDTNRKCAMLFNDQQAANVKGPGLYCVTKLNEKSGVFQDRLKSDTSLTVEYVEVHGGKVKVWHRYCKRTLGQNFASFLLSKKVEDKSHTASDDDDTISLYEVMLRGLLRSCPSSSTLIFPSDDCDENSEAWVNRDDGKLALSFLMSFSPASILSTLKNLKDKEVLEKVQLLQRYISQCDLTPDDEHLPIICERIKYKFFETHEKISSYNNIEDRLTTSGSSFFTLLRTKIEEDKEGILPGVLAIAAHQFQNDLAPDTKSNMFFVGILSEQGKHGMIKEVRELFGKNPDQTLIGDPYSLIAFDSCKLIGLQISPALKLMGSVLGPTCTAQELVDKARGYVMDIYWKNIVVPNMVLDCVEFGNAQNVSLCAGQDMALENSSDNVSIISGANVCCFGSCDPNPYSRI

SEQ ID NO: 69 - protein, 103 aa, Ehrlichia chaffeensis
MVMSYFDYILSYIINNPKYTALLVLVLLAGIAYLFKRKKSKSKYKKSTTSYTNNNSSSSTFTPKPHNKNYIPEHSTQHSQGSNFFVVESVSQVFESKMQGRSR

SEQ ID NO: 70 - protein, 184 aa, Ehrlichia canis
MGLYYNEYQNDHQIDFLIQQNGHSNVDIYHGTLHINGNEGKYYASASIVNDLTGDKGTINILNHGVKGIYSNESPKCDLEITLEHYDPASSDNIITSINKSFIVSVFSNLQDCLLIDSHNPLLMDPSKVEQINKEPVIAVLVHDGIDTKKIRDSFFNEKPENKEHKAGGCTYVYSHGKTETKCE

SEQ ID NO: 71 - protein, 641 aa, Ehrlichia canis
MQSDTHESIVRVDVSNGSNDEQLNDNSEFAKRTDKSTQKTKGSDRKSECLLKDIVESSEKTLVTESTSAYSQDIHDAEEFESRLSSSQWSTVQKGTRSTTKGDKEQKKFSGDGVKCLGKTEGVYSESVVESSAQRVSVPKGSIVENSSQWDKKNQDIKNSQLKLLKAENIAESGIISGSGEEWSVKFSKKDSKRNKKSQTFESDATVKAIDVSHASTVGEGKASQELSSKYLQQGARPKVRSHTQSISVGLPIGNANSKDVSQKKNVSLSTVNVSEMKAHSCMVTGIGTGVHLETKPIGRSGDTYGCVTNKQYLASVDKFKMSVEKFAKMHMQYIANIVFDVFAIENGNIIFRDEIQNVLLKTTKNIELCSFVIKQGIIMQVMSRVCSSKLYNSVMYCASHWDNQKILDAVLLEILNLGPNALVSMLPSMRNVFLNMHEGIEHNVFRLYSGGFYSGLLNMCYDFITQIDKSPGLMRLYNFIILSSQIHFGTMYDVICKSQVTAAGVQNVEAIKKLIFNGHRVGSVQICPTIFVHRCCGVKRTFRHLYSPNYKNVACTIGNLNIPSVIIKECSCNIDEKVAQCIEEKTMSFDVFVEDMVCNCVKSLLYQNVKLLLEKDIALYEKEVRSKYQTMGSTTSSRML

SEQ ID NO: 72 - protein, 461 aa, Ehrlichia canis
MLNRTQNDNITQTLQGANNSYSPEENANNLNIEEQLINNPEIQVNPNIQAEEVIADAIQEPYNPNAQIEAANATEDNIINDTIEENDNNLNIEEQLINNPEIQVNPNIQAEEVIADAIQEPYNPNAQIEAANVIEDNIIINNTIEENANNLNIEEQLINNPEIQVNPNIQAEEVIADAIQEPYNPNVQIEAANVIEDNIIINDTIEENANNLNIEEQLINNPEIQVNPNIQAEEVIADAMQEPDNPNAQIEAANATAITNINAMVTLQNINEIYPEDPESLITQEERAIEYMIQKREQVLAEANIYNDISRPRVLIDHSAAMQDILKGYSIAKTQNKINNIEQEILRNTIIQAEKNVGILIENRKDLHVNTTQEEPNAQIETDITDAIQQEELEEPSNQSNDISNNSSHVSSDNTETVEVDIEAIPSLLRRIARDIVALINTELNDPNAEHVNTQNQNINK

SEQ ID NO: 73 - protein, 242 aa, Ehrlichia canis
MSNTNDFQSIKDVIANIRKVMSSNDNGDSTTEQHDTNTQPDDTEHEVLNLENPENHVELHNIQHAHNTLNEINQTFQAISDLPITTYQNKTQPDIQISVKKEKIYPDQLSNVNQFISIEQSTQRLSSQERRLTTTHTLSEEISKNTSEIKGVHQKNTFISENLVSPESIVASSEEIKKLITQVHNYTKPTNVSSDKSPTIEELVINMLKPELSTWLNNNLQKLVKEIVEKEIKHIIKKSNQN

SEQ ID NO: 74 - protein, 217 aa, Ehrlichia canis
MIKTHDIAINTISAIMVTAAATGLALCSIGIIREHQYEYCALCVLLLLSACVSLIANYKKGISRKCASIRNYLSKISSDESLDGENKYYDPAPDLFAEHYFDEDAPSADRAEKIHVDRRQYYSEPYENEEQGQYSAYEEALHHSGWIRSQPFFAEYDTTPPDNRSAEMKTFSSSSSEAKVDTTIRGMQDKNNSKIQITDSRVEAVKPLSEEQGKGRA

SEQ ID NO: 75 - DNA, 24 bp, Artificial sequence
  Synthetic oligonucleotide
  modified_base (1)..(1): n = 5′-Iodo-2′-deoxyuridine
  misc_feature (1)..(1): n is a, c, g, t or u
ncgtcgacgatcggcggccgccgt

SEQ ID NO: 76 - protein, 7 aa, Artificial sequence
  Synthetic peptide
  misc_feature (2)..(2): Xaa can be any naturally occurring amino acid
  misc_feature (4)..(4): Xaa can be any naturally occurring amino acid
  misc_feature (6)..(6): Xaa can be any naturally occurring amino acid
RXRXRXR

SEQ ID NO: 77 - protein, 8 aa, Artificial sequence
  Synthetic peptide
LDAVTSIF

SEQ ID NO: 78 - protein, 6 aa, Artificial sequence
  Synthetic peptide
KHPMWC